(19) United States
(12) Patent Application Publication
(10) Pub. No.: US 2020/0156071 A1
(43) Pub. Date: May 21, 2020

(54) DEVICES AND METHODS FOR CELLULAR SECRETION ANALYSIS

(71) Applicant: THE UNIVERSITY OF BRITISH COLUMBIA, Vancouver, British Columbia (CA)

(72) Inventors: Carl Lars Genghis Hansen, Vancouver, BC (CA); Kathleen Lisaingo, Port Moody, BC (CA); Veronique Lecault, Vancouver, BC (CA)

(21) Appl. No.: 16/081,401

(22) PCT Filed: Mar. 17, 2017
(86) PCT No.: PCT/CA2017/050341

§ 371 Date: Aug. 30, 2018

Related U.S. Application Data
(60) Provisional application No. 62/309,663, filed on Mar. 17, 2016.

(51) Int. Cl. B01L 3/00; G01N 33/543; C12Q 1/04

(52) U.S. Cl. CPC B01L 3/502761; B01L 3/502707; G01N 33/54366; C12Q 1/04; B01L 2200/0647; B01L 2400/0403; B01L 2300/0809; B01L 2400/06; B01L 2200/12; B01L 3/50273

(57) ABSTRACT

Methods and devices for identifying a cell population comprising an effector cell exhibiting an extracellular effect are provided. The method comprises retaining in a plurality of open chambers a plurality of cell populations, each optionally comprising one or more effector cells. The open chambers can each comprise a readout particle population, and the open chambers are present in a first component of a device comprising a first component and optionally a second component. The open chambers have an average aspect ratio of ≥0.6 and the first component forms a reversible seal with the second component. The method further comprises incubating the plurality of cell populations or a subset thereof, and the one or more readout particles, or a subset thereof, within the chambers, assaying the cell populations for the presence of the extracellular effect, wherein the readout particle(s) provides a direct or indirect readout of the extracellular effect, and determining, based on the results of the assaying step, whether one or more cells within one or more cell populations of the plurality exhibits the extracellular effect.

### CROSS-REFERENCE TO RELATED APPLICATION

This application claims priority from U.S. Provisional Patent Application Ser. No. 62/309,663, filed Mar. 17, 2016, the disclosure of which is incorporated by reference in its entirety.

### BACKGROUND OF THE INVENTION

The cell is the fundamental unit of life and no two cells are identical. Indeed, seemingly identical clonal populations of cells have been shown to display phenotypic differences among cells within the population. Cellular differences exist across all levels of life, ranging from bacterial cells to partially differentiated cells (for example, adult stem and progenitor cells) to highly differentiated mammalian cells (for example, immune cells such as antibody secreting cells (ASCs)). Differences in cellular state, function and responses can arise from a variety of mechanisms including different histories, different differentiation states, epigenetic variations, cell cycle effects, stochastic variations, differences in genomic sequence, gene expression, protein expression and differing cell interaction effects, among others. Thus, the development of sensitive tools for assaying the properties of single cells is of great interest.

Many cell types secrete proteins that have biological function. For instance, different immune cells secrete a variety of factors including growth factors that act through cell signaling, cytokines that promote immune activation or suppression, and antibodies that specifically recognize pathogens including viruses, bacteria, cells, proteins, glycans, or other foreign challenges. Antibodies may also recognize non-foreign antigens (self-antigens or autoantigens) as part of anti-cancer responses or autoimmune conditions. As such, the ability to measure the amount of a protein that is secreted by a cell and/or the properties (e.g., sequence, binding partner) of a protein secreted by a cell is of high interest. Such measurements are ideally performed on individual cells so that measurements can be used to identify the differences in the properties of proteins secreted by any given cell, as compared to other cells within the same environment or population. For example, analysis of antibodies secreted by single or small numbers of ASCs can provide information regarding individual cells that is not available when the same analysis is performed on a bulk population (e.g., antibody binding properties, functional role).

The adaptive immune system of jawed vertebrates is capable of producing a large diversity of different antibodies. This diversity is generated through a variety of mechanisms including combinatorial recombination of variable region genes to create genetically encoded diversity in the heavy and light chain genes that are ultimately expressed as unique antibodies. In humans, this gene recombination occurs during B-cell development and can make billions of different antibodies in an individual. Following challenge, the B-cells that express an antibody that recognizes the challenge are activated and expanded to create clonally related B-cells. During this expansion, somatic hypermutation and selection results in the improved affinity of some antibodies in the population. The outcome of this process is a diverse set of B-cells that express many different antibodies. These B-cells either mature into plasma cells that continue to secrete antibodies into the medium, or into memory B-cells that express membrane-bound antibodies. To identify the antibodies in the population with improved properties, e.g., affinity, functional activity, individual antibodies and consequently ASCs, should be studied as individual cells.

The ability to efficiently identify antibodies that bind to a specific target, and/or a specific functional role, is of high interest for the generation of antibodies for use in research, diagnostics, and therapeutic development. The discovery of antibodies with optimal therapeutic properties, and in particular, antibodies that target surface receptors, remains a serious bottleneck in drug development. In response to immunization, an animal can make millions of different monoclonal antibodies (mAbs). Each mAb is produced by a single cell called an antibody-secreting cell (ASC), and each ASC makes only one type of mAb. Accordingly, antibody analysis, for example, for drug discovery purposes lends itself to single cell analyses. However, even if an ASC is analyzed individually, and not within a bulk population of cells, because a single ASC generates only a minute amount of antibody, when analyzed in the volume of conventional assay formats, the antibody is too dilute, making it completely undetectable.

Single cell analysis methods provide an approach to increase the speed, throughput, and efficiency of identifying new antibodies. A common theme in single cell antibody characterization methods is the use of small volume confinement to increase assay sensitivity. Once cells of interest are identified, they are recovered for subsequent analysis, including the sequencing of their antibody genes and/or the expression of larger amounts of antibody that can be used in subsequent experiments. Examples of miniaturized formats for the screening and selection of antibodies from single cells include plate-based methods (Czerkinsky et al. (1983). Journal of Immunological Methods 65(1-2), pp. 109-121; Leslie et al. (1996). Faseb Journal 10(6), pp. 346-346), microfluidic devices (Koster et al. (2008). Lab on a Chip 8(7), pp. 1110-1115; Mazutis et al. (2013). Nature Protocols 8(5), pp. 870-891; Singhal et al. (2010). Analytical Chemistry 82(20), pp. 8671-8679), microprinting (Loveet et al. (2006). Nature Biotechnology 24(6), pp. 703-707), and open microwell arrays (Jin et al. (2011). Nature Protocols 6(5), pp. 668-676; Wrammert et al. (2008). Nature 453(7195), pp. 667-U10).

Although techniques exist for single ASC analysis, they are still plagued by low throughput, sensitivity and the types of characterizations that can be carried out. The present invention addresses the need in the field for more robust devices, instruments and assays for performing analysis of single antibody secreting cells.

### SUMMARY OF THE INVENTION

The present invention, in one aspect, is directed to a platform for the analysis of an extracellular effect attributable to a single effector cell. The effector cell, in one embodiment, is a cell that secretes a biological factor, for example, an antibody (an ASC). The extracellular effect, in one embodiment, is cellular proliferation, decreased growth, apoptosis, lysis, differentiation, infection, binding (e.g., binding to a cell surface receptor or an epitope), morphology change, induction or inhibition of a signaling cascade, enzyme inhibition, viral inhibition, cytokine inhibition or activation of complement. The platform, in one embodiment, is a two-component microfluidic device comprising a first component with open microchambers, and a second component. The device in one embodiment, is a device depicted in one of FIGS. 4-8.

In one aspect, a method for identifying a cell population comprising an effector cell displaying an extracellular effect is provided. In one embodiment of this aspect, one or more populations of cells is analyzed within a two-component microfluidic device having a first component and a second component, or a one component device comprising open microchambers. The method comprises retaining individual cell populations, each optionally comprising one or more effector cells, in a plurality of different chambers located in the first component of the microfluidic device, wherein an individual cell population is retained in an individual chamber. Each chamber has an aspect ratio (defined as the height to the minimum lateral dimension) of ≥about 0.6, e.g., ≥0.7, ≥1, or ≥1 but ≤about 10. Alternatively, the average aspect ratio of each chamber of the device is of ≥about 0.6, e.g., ≥0.7, ≥1, or ≥1 but ≤about 10. The contents of one or more of the plurality of open chambers further comprise a readout particle population comprising one or more readout particles. The second component of the microfluidic device is brought into contact with the first component to create a reversible seal or a reversible partial seal between the two components. The contacting surface of either or both the first component and second component may include microscale structures, such as channel structures or chamber structures so that when such a seal is made, one or more of the open chambers of the first component of the device may be closed, or substantially closed. Moreover, two or more of the open chambers may be interconnected upon bringing the second component into contact with the first component, e.g., where a channel structure is present in the second component. The method further comprises incubating the plurality of cell populations or a subset thereof, and the one or more readout particles, or a subset thereof, within the one or more plurality of open chambers, assaying the plurality of cell populations or subset thereof for the presence of the extracellular effect, wherein the readout particle population or a subpopulation thereof provides a direct or indirect readout of the extracellular effect, and determining, based on the results of the assaying step, whether one or more effector cells within the one or more cell populations of the plurality exhibits the extracellular effect.

In one embodiment, the effector cell is a cell that secretes a biological factor, e.g., an antibody. It is not necessary that the specific effector cell or effector cells, displaying the extracellular effect be initially identified so long as the presence of the extracellular effect is detected within a specific chamber. That is, some or all the cells in the chamber where the effect is measured can be recovered if desired for further characterization to identify the specific cell(s) displaying the extracellular effect.

### BRIEF DESCRIPTION OF THE DRAWINGS

FIG. 1 a process flow diagram for one embodiment for single effector cell identification and selection. Single cells are obtained from any animal or from a population of cells in culture, and are optionally enriched for an effector cell population. High-throughput microfluidic analysis using two-component microfluidic devices is used to perform functional screens on antibodies secreted from single effector cells, in some cases, present in heterogeneous cell populations. After one or multiple rounds of analysis, cells are recovered and antibody variable region genes are amplified for sequencing (Vh/Vl) and/or cloning into cell lines. This process allows for the screening of from about 100,000 cells to about 100,000,000 cells in a single device run, with sequences recovered one week later.

FIG. 2 is a process flow diagram for one embodiment of a microfluidic effector cell enrichment method. Effector cells are first loaded at an average concentration of 25 cells per chamber and incubated to create polyclonal mixtures of antibodies. Screening of polyclonal mixtures, created with or without a step to culture the assayed cells, is used to identify chambers displaying an extracellular effect (e.g., binding, affinity, or functional activity). Forty positive chambers are then recovered to achieve an enriched population with ˜4% of effector cells making antibodies of interest. The effector cells of the enriched population are then analyzed in a second array at limiting dilution to select a single ASC(s) displaying the extracellular effect. The time required for enrichment is about 4 hours and total screening throughput is from about 100,000 cells to 100,000,000 per run. Enrichment process may be performed twice if needed, and may use the same or different properties for each screen.

FIG. 3 is an alignment of the extracellular domain for PDGFRα across human, rabbit, mouse and rat. (Top) Ribbon diagram showing structure of extracellular domain (ECD) of two PDGFRβ in complex with a dimer of PDGFBB (from Shim et al. (2010). Proc. Natl. Acad. Sci. U.S.A. 107, pp. 11307-11312, incorporated by reference herein in its entirety). Note, a PDGFRβ is shown since a similar structure for PDGFRα is expected but was not available. (Bottom) Alignment of ECD for PDGFRα across human, mouse, rabbit, rat. Regions of variation from the human isoform are denoted by lighter shading and “*”. The substantial variation indicates there are numerous epitopes available for antibody recognition, with rabbit having the most variation from human.

FIG. 4 is a schematic of a cross section of one device embodiment 400 provided herein. This device is referred to herein as a split device, or multicomponent device, with a multilayer top. The top component 401 includes push down valve structures 404 and an open channel 405. The bottom component 402 includes open chambers 403.

FIG. 5 are cross section schematics of device embodiments 500 and 500′.

FIG. 6 is a schematic of a cross section of two-component device embodiment 600, including a single layer top component 601, which is translatable. The top schematic is a cross section of the device where flow to the chambers in the bottom component of the device is open. The bottom schematic shows sealed chambers via translation of the top component.

FIG. 7 is a schematic of a cross section of one device embodiment 700 provided herein. This device is referred to herein as a split device, or multicomponent device, with a single layer unpatterned top.

FIG. 8 is a schematic of a cross section of one device embodiment provided herein. This device includes an open chamber array with volume extrusion for imaging.

FIG. 9 is a schematic diagram of a microfluidic chamber according to an embodiment of the invention illustrating the use of a magnetic field to position particles within the chamber.

FIG. 10 is a schematic of single cell HV/LV approach using template-switching. Single cells are deposited into microfuge tubes and cDNA is generated from multiplexed gene-specific primers targeting the constant region of heavy and light chains. Template-switching activity of MMLV enzyme is used to append the reverse complement of a template-switching oligo onto the 3′ end of the resulting cDNA. Semi-nested PCR, using multiplexed primers that anneal to the constant region of heavy and light chain and a universal primer complementary to the copied template switching oligo, is used to amplify cDNA and introduce indexing sequences that are specific to each single cell amplicon. Amplicons are then pooled and sequenced.

FIG. 11 shows top (right) and cross-sectional (left) diagrams of a method of identifying an effector cell that produces a biomolecule capable of specifically binding a target readout particle according to an embodiment.

FIG. 12 shows top (right) and cross-sectional (left) diagrams of a method of identifying at least one effector cell that produces a biomolecule (e.g., antibody) that binds specifically to malignant cells but not normal cells.

FIG. 13 shows top (right) and cross-sectional (left) diagrams of an embodiment of a method of identifying an effector cell that produces a biomolecule that binds to a readout cell where a subpopulation of effector cells are functionalized to also act as readout cells.

FIG. 14 is a schematic diagram of an antibody tetramer.

FIG. 15 shows top (right) and cross-sectional (left) diagrams of a method of screening for a target epitope/molecule to which a known biomolecule binds according to an embodiment.

FIG. 16 shows top (right) and cross-sectional (left) diagrams of a method of identifying an effector cell which produces an antibody that specifically binds a target epitope/antigen according to an embodiment.

FIG. 17 shows top (right) and cross-sectional (left) diagrams of a method of quantifying cell lysis.

FIG. 18 shows top (right) and cross-sectional (left) diagrams of a method of identifying the presence of an effector cell which produces an antibody that specifically binds a target epitope/antigen according to an embodiment.

FIG. 19 shows top (right) and cross-sectional (left) diagrams of a method of quantifying cell lysis.

FIG. 20 shows top (right) and cross-sectional (left) diagrams of a method of identifying an effector cell which produces a biomolecule that induces growth of readout cells.

FIG. 21 shows top (right) and cross-sectional (left) diagrams of a method of identifying the presence of an effector cell which produces a biomolecule that stimulates readout cells to undergo apoptosis.

FIG. 22 shows top (right) and cross-sectional (left) diagrams of a method of identifying an effector cell which produces a biomolecule that stimulates autophagy in readout.

FIG. 23 shows top (right) and cross-sectional (left) diagrams of a method of identifying an effector cell which produces a biomolecule that neutralizes a cytokine.

FIG. 24 shows top (right) and cross-sectional (left) diagrams of a method of identifying an effector cell which produces a biomolecule that inhibits cellular virus infection.

FIG. 25 shows top (right) and cross-sectional (left) diagrams of a method of identifying the presence of an effector cell which produces a biomolecule that inhibits the function of a target enzyme according to an embodiment of the invention.

FIG. 26 shows top (right) and cross-sectional (left) diagrams of a method of identifying an effector cell that displays a molecule that activates a second type of effector cell, which in turn secretes molecules that have an effect on a readout particle.

FIG. 27 shows top (right) and cross-sectional (left) diagrams of a method of identifying an effector cell that secretes a molecule that activates a second type of effector cell, which in turn secretes molecules that have an effect on a readout particle.

FIG. 28 shows top (right) and cross-sectional (left) diagrams of a method to detect an effector cell secreting an antibody with high affinity, present in a heterogeneous population of cells containing cells that secrete an antibody for the same antigen but with lower affinity.

FIG. 29 shows top (right) and cross-sectional (left) diagrams of a method of screening for antibodies with increased specificity for an antigen according to an embodiment of the invention in which readout particles displaying different epitopes are distinguishable by different optical characteristics.

FIG. 30 shows top (right) and cross-sectional (left) diagrams of a method of simultaneously (i) identifying a cell secreting a biomolecule in a homogeneous or heterogeneous population of effector cells and (ii) analyzing one or more intracellular compounds affected by the molecule.

FIG. 31 is a schematic of an instrument according to one embodiment of the invention. The instrument can be used together with one of the devices and/or methods described herein, e.g., for identifying a cell population comprising an effector cell having an extracellular effect.

FIG. 32 is a set of optical micrographs showing Tango™ CXCR4-bla U2OS cells after loading through channels of a one-component, multilayered microfluidic device fabricated via MSL. Scale bar: 100 μm.

FIG. 33 is a set of optical micrographs showing Tango™ CXCR4-bla U2OS cells 12 hours after loading into the bottom chamber of a two-component microfluidic device. Scale bar: 34 μm.

FIG. 34, left, are streamlines resulting from flow through chamber with dimensions of 100 μm×100 μm×150 μm (depth×width×length) and having an aspect ratio of 1.0. Stream lines penetrate to the bottom of the chamber and cause movement of particles and/or loss of particles at high flow rates. FIG. 34, right, are streamlines of flow through a cylindrical chamber have a depth of 125 μm, a diameter of 100 μm, and an aspect ratio of 1.25. Streamlines exhibit a recirculating vortex in the bottom half of the chamber. Chambers are not drawn to scale.

FIG. 35 are optical micrographs of microfluidic chambers after loading with ASCs from immunized animals (left panel). ASCs were co-incubated with CXCR4 expressing cells, along with parental cell lines (negative control, 3rd panel from left). Antibodies specific for CXCR4 were detected (2nd panel from left). Antibodies not specific for CXCR4 binding both CXCR4 expressing and parental cells lines were also detected and excluded from the analysis (right panel). Scale bar: 34 μm.

FIG. 36 are optical micrographs showing microbeads coated with 3 different influenza related antigens: H1N1 (10 μm beads), H3N2 (5 μm beads) and B strain (3 μm beads). After loading human B-cells and incubating to accumulate secreted antibodies, the specific antibody binding of antibodies to the different bead types was detected using a mixture of fluorescently labelled secondary anti-human antibodies, with different secondary antibodies labeled in different colors and each being specific to the detection of a different isotype, IgG, IgA and IgM. Human IgGs are shown for 3 different cells, secreting antibodies specific to H1N1, H3N2 and B strain antigens.

FIG. 37 shows the feasibility of detection and selection of antigen specific antibodies using a multistep extracellular effect assay. Optical micrographs of microfluidic chambers are provided. Antibodies specific for m4-1BB and h4-1BB were probed for. h4-1BB Ligand labeled with Dylight-650 was also incubated in the chambers to detect the possible blocking of binding of the ligand to its natural receptor in the presence of target specific antibodies.

FIG. 38 shows a panel of chambers following detection of antigen-specific antibodies with a fluorescently labeled secondary antibody. The histograms of pixel intensity are shown for the center chamber that contains the ASC, as well as the adjacent top, bottom, right, and diagonal (top-right) chambers. Histograms are labeled to indicate the pixels arising from positive bead signal and show that the center chamber has more pixels above the background fluorescence and that these pixels have a higher total intensity.

FIG. 39 are optical micrographs showing ASCs isolated from human samples incubated with live bacteria (Klebsiella pneumoniae). Examples of IgG and IgA binding are demonstrated from human tonsils and human bone marrow.

FIG. 40 shows a series of time-lapse images illustrating the growth of K562 cells in different micro-chambers of a microfluidic device. Scale bar: 100 μm.

FIG. 41 is an agarose gel separating twenty PCR reactions generated from the capillary-recovered consecutive individual microfluidic chambers containing an ASC, or no ASC. Ten upper reactions (H) were generated with the antibody heavy-chain specific primers, and ten lower reactions (L) generated with the antibody light-chain specific primers.

### DETAILED DESCRIPTION OF THE INVENTION

As used herein, the singular forms “a”, “an” and “the” include plural referents unless the context clearly dictates otherwise.

“Readout,” as used herein, refers to the method by which an extracellular effect is reported. “Readout particle,” as used herein, means any particle, including a bead or cell, e.g., a functionalized bead or a cell that reports a functionality or property, or is used in an assay to determine an extracellular effect (e.g., functionality or property) of an effector cell, or a product of an effector cell such as an antibody. A “readout particle” can be present as a single readout particle or within a homogeneous or heterogeneous population of readout particles within a single assay chamber. A “readout particle population” comprises one or more readout particles. In one embodiment, a readout particle is a bead functionalized to bind one or more biomolecules secreted by an effector cell (e.g., one or more antibodies), or released by an effector or accessory cell upon lysis. A single readout particle may be functionalized to capture one or more different types of biomolecules, for instance a protein and/or nucleic acid. The one or more proteins, in one embodiment, is one or more different monoclonal antibodies. In one embodiment, the readout particle is a bead or a cell that is capable of binding antibodies produced by an effector cell that produces and/or secretes antibodies. In some embodiments, an effector cell may also be a readout particle, e.g., where a secretion product of one effector cell in a population has an effect on a larger, or different, sub-population of the effector cells or, alternatively, where the secretion product of one effector cell is captured on the same cell for readout of the capture.

A “readout cell,” as used herein, is a type of readout particle that exhibits a response in the presence of a single effector cell or a cell population comprising one or more effector cells, for example one or more effector cells that secrete antibodies. In various embodiments, the readout cell is a cell that displays a surface antigen or a receptor (e.g., GPCR or RTK or ion channel). In one embodiment, the binding of the secreted molecule to a readout cell is the assayed extracellular effect. The readout cell may be fluorescently labeled and/or possess fluorescent reporters that are activated upon binding.

An “effector cell,” as used herein, refers to a cell that can exert an extracellular effect. The extracellular effect is a direct or indirect effect on a readout particle. The extracellular effect is attributable to the effector cell, or a molecule secreted by the effector cell, for example a signaling molecule, metabolite, an antibody, neurotransmitter, hormone, enzyme, cytokine. In one embodiment, the effector cell is a cell that secretes a protein such as an antibody, or displays a protein, such as a T-cell receptor. In embodiments described herein, the extracellular effect is characterized via the use of a readout particle or a readout particle population or subpopulation. For example, in one embodiment, the extracellular effect is the agonizing or antagonizing of a cell surface receptor, ion channel or ATP binding cassette (ABC) transporter, present on a readout cell or readout bead. In one embodiment, the effector cell is an antibody secreting cell (ASC). The present invention is not limited by effector cell type or cell population that can be assayed according to the methods of the present invention. Examples of types of effector cells for use with the present invention include primary antibody secreting cells from any species (e.g., human, mouse, rabbit, etc.), primary memory cells (e.g., IgG, IgM, IgD or other immunoglobins displayed on surface of cells or expand/differentiate into plasma cells), dendritic cells that display protein or peptides on their surface, hybridoma fusions either after a selection or directly after fusion, a cell line that has been transfected (stable or transient) with one or more libraries of monoclonal antibodies (mAbs) (e.g., for affinity maturation of an identified mAb using libraries of mutants in fab regions or effector function optimization using identified mAbs with mutations in Fc regions or cell lines transfected with heavy chain (HC) and light chain (LC) combinations from amplified HC/LC variable regions obtained from a person/animal/library or combinations of cells expressing mAbs (either characterized or uncharacterized to look for synergistic effects). Other effector cells include T-cells (e.g., CD8+ T-cell, and CD4+ T-cell), hematopoietic cells, cell lines derived from humans and animals, recombinant cell lines, e.g., a recombinant cell line engineered to produce antibodies, a recombinant cell line engineered to express a T-cell receptor.

An “antibody secreting cell” or “ASC”, is any cell type that produces and secretes an antibody. Plasma cells (also referred to as “plasma B-cells,” “plasmocytes” and “effector B-cells”) are terminally differentiated, and are one type of ASC. Other ASCs include plasmablasts, cells generated through the expansion of memory B-cells, cell lines that express recombinant monoclonal antibodies and hybridoma cell lines. In another embodiment, the effector cell is a cell that secretes a protein other than an antibody.

“Extracellular effect”, as used herein, is a direct or indirect effect on a readout particle that is extracellular of an effector cell, which in many embodiments, is an antibody secreting cell (ASC), including but not limited to increased cellular proliferation, decreased growth, apoptosis, lysis, differentiation, infection, binding (e.g., binding to a cell surface receptor or an epitope), morphology change, induction or inhibition of a signaling cascade, enzyme inhibition, viral inhibition, cytokine inhibition, activation of complement. The extracellular effect in one embodiment, is a binding property of a biomolecule of interest, secreted by an effector cell, to a target. In another embodiment, the extracellular effect is a response such as apoptosis of a second cell. The measured extracellular effect in one embodiment, is different (i.e., a variation), as compared to the effect displayed by a second effector cell or cell population comprising the second effector cell, or as compared to a control (negative or positive control).

A “heterogeneous population” as referred to herein, particularly with respect to a population of particles or cells, means a population of particles or cells that includes at least two particles or cells that have a differing feature. The feature, in one embodiment, is morphology, size, fluorescent reporter, cell species, phenotype, genotype, cell differentiation type, the sequence of one or more expressed RNA species or a functional property.

“Coating” as used herein, may be any addition to a chamber or channel surface, which either facilitates or inhibits the ability of an effector cell, a secreted molecule such as an antibody, a readout particle or an accessory particle to adhere to a surface of the surface, or facilitate a biological process such as cell growth. The coating of a surface, in one embodiment, is a surface functionalization. The coating, in one embodiment, is selected from: a cell; a polymer brush, a polymer hydrogel, self assembled monolayers (SAM), photo-grafted molecules, a protein or protein fragment having cell binding properties (for example, a cell binding domain from actin, fibronectin, integrin, protein A, protein G, etc.), poly-L-lysine. In one embodiment, arginine-glycine-aspartate-(serine) (RGD(S)) peptide sequence motif is used as the coating. In another embodiment, poly-lysine is used as a polymer coating with PDMS to enhance cell adhesion via electrostatic interactions; a phospholipid having cell binding properties, a cholesterol having cell binding properties, a glycoprotein having cell binding properties or a glycolipid having cell binding properties is employed. In another embodiment, a PDMS surface is functionalized using biotinylated biomolecules. Bovine serum albumin (BSA) can also be employed as a coating. Due to its hydrophobic domains, BSA readily adsorbs via hydrophobic effect on hydrophobic PDMS surfaces enabling further direct coupling of streptavidin based conjugates in the chambers (protein, DNA, polymers, fluorophores). Polyethylene glycol based polymers are also known for their bio-fouling properties and can be coated on PDMS surface (adsorption, covalent grafting), preventing cell adhesion. Poly(paraxyxlylene), e.g., parylene C can also be deposited using chemical vapor deposition (CVD) on PDMS surfaces and prevent cellular adhesion.

“Isolated,” as used herein, refers to the circumstances under which a given chamber does not permit substantial contamination of an effector cell and/or readout particle being analyzed with a particle(s) or biomolecule(s) of another chamber of the microfluidic device. Such isolation may be achieved, for example, by sealing a chamber or a set of chambers in the case of compound chambers, by limiting fluid communication between chambers or by restricting fluid flow between chambers, e.g., by specific chamber architectural features such as aspect ratio.

A “microfluidic device,” as used herein refers to a device used for the manipulation of fluids that comprises features with a minimum dimension of less than 1 mm.

“Aspect ratio”, unless otherwise specified, refers to the ratio of the height/depth of a microfluidic feature, e.g., a chamber, to the minimum lateral dimension.

Although microfluidic devices are useful for addressing problems of throughput and single cell sensitivity, in many instances, the loading of cells into a device through microscale channels can be challenging. Larger cell types, or cells that naturally adhere to channel surfaces, can clog the channels prior to being delivered to assay chambers of the device, and therefore impede analysis and lead to device failure. Inefficient loading of cells into the device or uneven loading of cells across the device can also occur when cells are loaded into chambers through microchannels. Further, during the loading of cells through microchannels there may be a substantial number of cells that “settle” into the ports at the entrance of the device and that subsequently do not enter the analysis chambers. These problems are particularly acute when trying to standardize and automate cell loading and analysis in a robust instrument. In this case, the operator should not be required to adjust loading parameters in real time. Further, cell loading can be problematic when working with adherent cells types or other large cell types that may be required for a variety of different cell-based antibody discovery assays.

The devices and instruments provided herein address this cell loading problem by providing alternative assay formats amenable for use with a vast array of cell types.

Methods and apparatuses known in the art which are designed to accommodate more than a single cell have limitations that make them unsuitable for the types of assays described herein. For example, maintaining cell viability, selectively recovering effector cells of interest, limiting/impeding evaporation within a device, maintaining substantially the same pressure, eliminating cross-contamination can all be problematic. Known device architectures also limit imaging capabilities (e.g., by providing particles in different focal planes, reduced resolution) and lack throughput of the present invention (WO 2012/072822 and Bocchi et al. (2012), each incorporated by reference in their entireties for all purposes).

In aspects and embodiments described herein, the devices and assays of the invention include medium wash steps and/or co-culture with different cell types. This format provides several advantageous features that may be used for performing a wide array of single cell antibody screening assays. Using a multistep screening strategy, such devices and assays can be used to screen hundreds of thousands of cells per device run. The methods described herein are compatible with high-resolution microscopy which supports a wide array of assay formats. Further, the devices and assay formats described herein provide a means to immobilize antibody secreting cells (ASCs) for analysis while still enabling controlled exchanges of medium components, thereby supporting assays that utilize one or more medium exchange steps. Importantly, the ability to exchange medium also enables the prolonged culture of antibody secreting cells and other cell types. Notably, the devices and assays of the invention provide a means to concentrate secreted biomolecules such as antibodies, as well as to achieve high spatial density in analysis.

Besides addressing the cell loading problem described above, the devices and methods provided herein provide advantages for assessing an extracellular effect of a single cell, for example, an extracellular effect of an antibody secreted by a single ASC. For example, the devices described herein are scalable, enable reduced reagent consumption and increased throughput to provide a large single cell assay platform for studies that would otherwise be impractical or prohibitively expensive.

Without wishing to be bound by theory, the concentration enhancement and rapid diffusive mixing afforded by the nanoliter assay volumes provided herein, along with precise cellular handling and manipulation (e.g., spatio-temporal control of medium conditions) enables the single cell analysis of effector cells such as immune cells (e.g., B-cells, T-cells, and macrophages) whose primary functions include the secretion of different effector proteins such as antibodies and cytokines and chemokines.

Embodiments described herein provide instruments, devices and methods capable of performing multicellular assays from a plurality of cell populations, each comprising one or more effector cells, followed by recovery of one or more of the plurality of cell populations for subsequent analysis. The multicellular assays, in one embodiment, are assays of secretion products, e.g., antibodies, of one or more cells in a population. In some embodiments, the cell populations are heterogeneous cell populations. That is, two or more cells in a population differ in genotype, phenotype, different recombined antibody genes, different recombined T-cell receptor genes, or some other property. Moreover, where multiple cell populations are assayed in parallel on one device, in one embodiment, at least two of the populations are heterogeneous with respect to one another (e.g., different number of cells, cell type, etc.). In some assay embodiments, a readout particle population comprising one or more readout particles, which serve as detection reagents (e.g., readout cells expressing a cell receptor, readout bead, sensor, soluble enzyme, etc.) are exposed to individual cell populations comprising one or more effector cells, and secreted products from the one or more effector cells, at a sufficient concentration for a readout signal (e.g., a fluorescent signal) to be detected. The readout signal reports a property of the effector cell, e.g., a biological response/extracellular effect (e.g., apoptosis) induced by one or more the effector cells in the population on one or more readout particles.

Notably, because effector cells are in many cases rare cells, not all cell populations assayed by the methods described herein necessarily contain an effector cell. For example, where thousands of cell populations are assayed on a single device, in one embodiment, only a fraction of the cell populations comprise an effector cell.

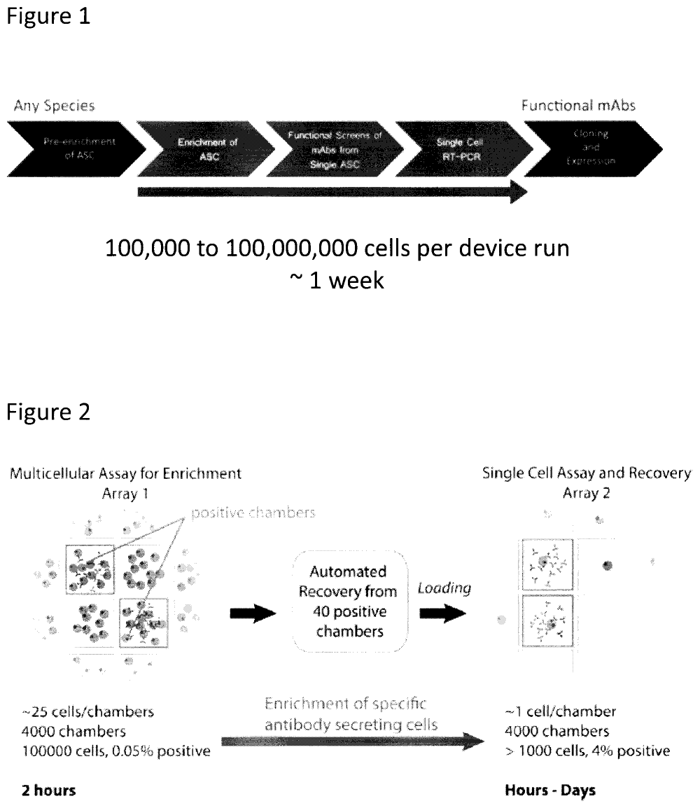

The devices and assays described herein provide single cell assays whereby one or more effector cells are present in one or more individual cell populations, in single assay chambers of a device. Importantly, effects of a single effector cell can be detected within a larger cell population, for example a heterogeneous cell population. By taking the multicellular assay approach within a single assay chamber, the embodiments described herein can operate at much greater throughput over what has been reported previously for single cell assays. For example, arrays of 1,000,000 chambers may be fabricated on a practical substrate size and used with loading densities of up to 100 cells per chamber, thereby achieving screening throughputs of up to 100,000,000 cells per experiment (FIG. 1). Once a cell population is identified as exhibiting an extracellular effect on a readout particle (e.g., a variation in an extracellular effect as compared to another population or a control value), the cell population, in one embodiment, is recovered and further assayed as individual cell subpopulations (e.g., the recovered cell population is assayed at limiting dilution) to determine which effector cell(s) within the population is responsible for the extracellular effect.

The present invention harnesses the small reaction volumes and massively parallel analysis capabilities to assay individual cell populations (which may include a single cell or a small number of cells) for a property of interest, i.e., an “extracellular effect.” The extracellular effect, in one embodiment, is a property of an antibody secreted by a cell present in a cell population. The extracellular effect is not limited to a particular effect, rather, it may be a binding property (specificity, affinity, Kₒₙ, K_{off}, etc.) or a functional property, for example agonism or antagonism of a cell surface receptor. In one embodiment, the extracellular effect is an effect exerted by a secretion product of an effector cell (e.g., antibody). However, the extracellular effect may be an effect of the cell itself, for example an effect exerted by a cell surface protein of the effector cell.

The devices and methods provided herein are based in part on the concept that small is sensitive. The devices provided herein comprise many thousands, tens of thousands, or hundreds of thousands, of nanoliter volume cell analysis chambers, each having a volume approximately from about 10,000 to about 100,000 times smaller than conventional plate-based assays. In these nanoliter or sub-nanoliter chambers, each single effector cell (e.g., ASC) produces high concentrations of secreted biomolecule, e.g., antibodies within minutes. This concentration effect, in one embodiment, is harnessed to implement cell-based screening assays that identify antibodies, made by single primary ASCs, with a specific functional property(ies), such as the modulation (e.g., agonism or antagonism) of cell surface receptor activity. The concentration effect also allows for the identification of single effector cells demonstrating a property of interest in the presence of other effector cells and non-effector cells that do not demonstrate the property. Functional extracellular effect assays amenable for use with the methods and devices provided herein are described in detail herein.

Importantly, in the assays provided herein, it is not necessary that a specific effector cell or subpopulation of effector cells, having the particular property be initially identified so long as the presence of the extracellular effect is detected within a particular chamber comprising a cell population. Some or all the cells within the chamber where the effect is measured can be recovered for further characterization, e.g., at limiting dilution, to identify the specific cell or cells responsible for the extracellular effect. In embodiments described herein, an extracellular effect from a single cell can be detected in the presence of other cells in the same chamber, e.g., from about 2 to 300 other cells, e.g., from about 2 to about 250 cells in the same chamber, from about 2 to about 150 cells in the same chamber, from about 2 to about 100 cells in the same chamber, from about 2 to about 50 cells in the same chamber, from about 2 to about 30 cells in the same chamber, from about 2 to about 20 cells in the same chamber, or from about 2 to about 10 cells in the same chamber.

Provided herein are devices designed to carry out extracellular effect assays. Such assays allow for the detection of a single effector cell of interest present, in a heterogeneous cell population, or as a single cell, in an assay chamber of one of the devices described herein. Specifically, in the case where a chamber contains a heterogeneous cell population, where one or more of the cells in the population secretes antibodies (i.e., a heterogeneous ASC population within a single reaction chamber), whereby only one effector cell or a subpopulation of effector cells secretes an antibody that produces a desired extracellular effect, the embodiments described herein provide a device and method for qualitatively and/or quantitatively detecting the extracellular effect. Once a chamber is identified that exhibits the effect, the cell population from the chamber is recovered for downstream analysis, for example, by dividing the cell population into subpopulations at limiting dilution. In one embodiment, one or more heterogeneous populations of cells displaying an extracellular effect, are recovered and subjected to further screening at limiting dilution, to determine which cell the extracellular effect is attributable to.

One embodiment of a work flow for a single cell antibody secreting cell (ASC)/antibody selection pipeline is shown in FIG. 1. In this embodiment, a host animal is immunized with a target antigen and cells are obtained from spleen, blood, lymph nodes and/or bone marrow one week following a final immunization boost. These samples are then optionally enriched for ASCs by flow cytometry (e.g., FACS) or magnetic bead purification using established surface markers (if available) or using microfluidic enrichment. The resulting ASC enriched population is then loaded into a device array comprising nanoliter volume chambers, with a loading concentration chosen to achieve from about one to about 250 cells per chamber, e.g., from about 2 to about 100 cells per chamber, from about 10 to about 100 cells per chamber, or from about 10 to about 50 cells per chamber. In one embodiment, greater than 80% of the chambers of a device contain a cell population. As described further herein, cell loading is achieved by directly loading into open chambers in a bottom component of a device, e.g., by hydrostatic pressure created by a liquid column, by creating a flow using a dispensing instrument such as a pipette, or by exchanging the medium overtop of the bottom component and moving the top component or bottom component up and down to evoke a fluid transfer to the microchambers.

Depending on if and how a pre-enrichment is carried out, individual chambers will comprise multiple ASCs, single ASCs or zero ASCs. Cells are incubated in chambers to allow for antibodies to be secreted in the chamber volume. Because ASCs typically secrete antibodies at a rate of 1000 antibody molecules per second, and the volume of individual chambers provided herein in one embodiment, are on the order of about 2 nL to about 5 nL, a concentration of about 10 nM of each secreted monoclonal antibody is provided in about 3 hours (each unique ASC secretes a unique monoclonal antibody). In a further embodiment, delivery and exchange of reagents in chambers is employed to implement image-based extracellular effect assays, which are read out using automated microscopy and real-time image processing. Reagents can be exchanged by hydrostatic pressure created by a liquid column, by creating a flow overtop the array of microchambers using a dispensing instrument such as a pipette, or by exchanging the medium overtop of the bottom component and moving the top component or bottom component up and down to evoke a fluid transfer to the chambers.

Individual ASCs or cell population comprising one or more ASCs that secrete antibodies with desired properties (e.g., binding, specificity, affinity, function) are then recovered from individual chambers. Further analysis of the recovered cell populations at limiting dilution is then carried out, e.g., via a benchtop method or in another microfluidic device provided herein (see, e.g., FIG. 2). Alternatively, nucleic acid can be sequenced from a cell population in a single sequencing reaction, to determine antibody sequences. In the case that an individual ASC is provided to a chamber and recovered, further analysis of the individual ASC can also be carried out. For example, in one embodiment, the further analysis includes single cell RT-PCR to amplify paired HV and LV for sequence analysis and cloning into cell lines.

In another embodiment, after an animal is immunized and cells are obtained from spleen, blood, lymph nodes and/or bone marrow, the cells make up a starting population that are loaded directly into individual chambers of a device, i.e., as a plurality of cell populations, wherein individual cell populations are present in each chamber. An extracellular effect assay is then carried out in the individual chambers to determine if any of the individual cell populations comprise one or more effector cells responsible for an extracellular effect.

Although a host animal can be immunized with a target antigen prior to carrying out an extracellular effect assay, the invention is not limited thereto. For example, in one embodiment, cells are obtained from spleen, blood, lymph nodes or bone marrow from a host (including human) followed by an enrichment for ASCs. Alternatively, no enrichment step takes place and cells are directly loaded into chambers of a device provided herein, i.e., as a plurality of cell populations, where individual cell populations are present in each chamber.

The methods provided herein allow for the selection of antibodies from any host species. This provides two key advantages for the discovery of therapeutic antibodies. First, the ability to work in species other than mice and rats allows for the selection of mAbs to targets with high homology to mouse proteins, as well as mAbs to human proteins that cross-react with mice and can thus be used in easily accessible pre-clinical mouse models. Second, mouse immunizations often result in responses that feature immunodominance to a few epitopes, resulting in a low diversity of antibodies generated; expanding to other species thus greatly increases the diversity of antibodies that recognize different epitopes. Accordingly, in embodiments described herein, mice rats and rabbits are used for immunizations, followed by the selection of ASCs from these immunized animals. In one embodiment, a rabbit is immunized with an antigen, and ASCs from the immunized rabbit are selected for with the methods and devices provided herein. As one of skill in the art will recognize, rabbits offer advantages of a distinct mechanism of affinity maturation that uses gene conversion to yield greater antibody diversity, larger physical size (more antibody diversity), and greater evolutionary distance from humans (more recognized epitopes).

The immunization strategy, in one embodiment, is a protein, cellular, and/or DNA immunization. For example, for PDGFRα, the extracellular domain obtained from expression in a mammalian cell line, or purchased from a commercial source (Calixar) is used. For CXCR4, in one embodiment, virus-like particle (VLP) preparations, a nanoparticle having a high expression of GPCR in native conformation, from a commercial source (Integral Molecular) is used. Cell-based immunization is performed by overexpression of full-length proteins in a cell line (e.g., 32D-PDGFRα cells for mice/rats, and a rabbit fibroblast cell line (SIRC cells) for rabbits; including protocols in which a new cell line is used in the final boost to enrich for specific mAbs. A variety of established DNA immunization protocols are amenable for use with the present invention. DNA immunization has become the method of choice for complex membrane proteins since it (1) eliminates the need for protein expression and purification, (2) ensures native conformation of the antigen, (3) reduces the potential for non-specific immune responses to other cell membrane antigens, and (4) has been proven effective for challenging targets (Bates et al. (2006). Biotechniques 40, pp. 199-208; Chambers and Johnston (2003). Nat. Biotechnol. 21, pp. 1088-1092; Nagata et al. (2003). J. Immunol. Methods 280, pp. 59-72; Chowdhury et al. (2001). J. Immunol Methods 249, pp. 147-154; Surman et al. (1998). J. Immunol. Methods 214, pp. 51-62; Leinonen et al. (2004). J. Immunol. Methods 289, pp. 157-167; Takatasuka et al. (2011). J. Pharmacol. and Toxicol. Methods 63, pp. 250-257, each incorporated by reference in their entireties for all purposes). Immunizations are performed in accordance with animal care requirements and established protocols.

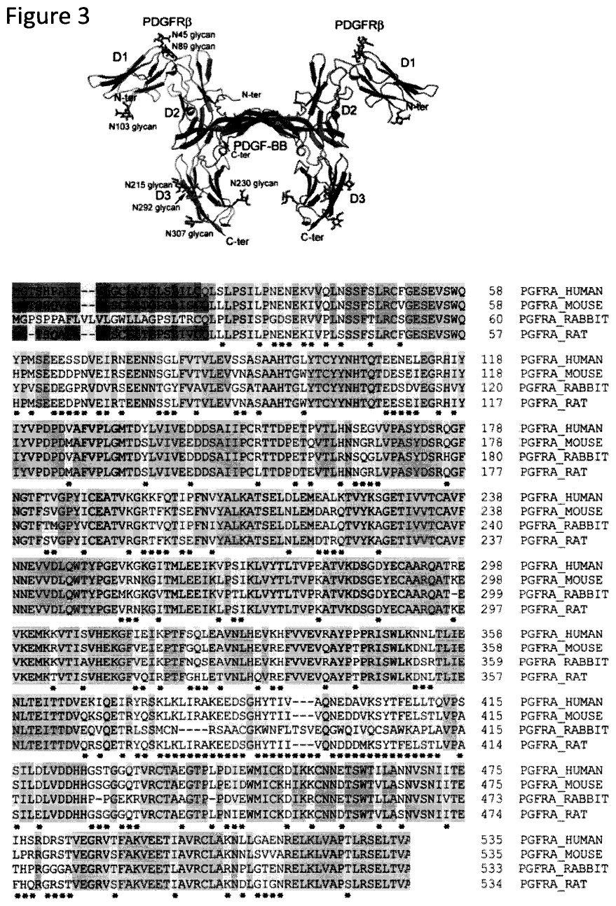

Anti-PDGFRα antibodies have been previously produced in rats, mice, and rabbits, and comparison of the extracellular domain of PDGFRα shows several sites of substantial variation (FIG. 3). Thus, it is expected that a good immune response is obtainable from this antigen. Anti-CXCR4 mAbs have also been previously generated using both lipoparticles and DNA immunizations, so this target is also likely to yield a good immune response. In one embodiment, the co-expression of GroEl or GM-CSF (either co-expressed or as a fusion) as a molecular adjuvant will be used. Alternatively, adjuvants and immunization schedules disclosed in Takatsuka et al. (2011). J. Pharmacol. and Toxicol. Methods 63, pp. 250-257; and/or Fujimoto et al. (2012) J. Immunol. Methods 375, pp. 243-251, incorporated by reference in their entireties for all purposes, are employed.

The present invention is directed in part to device architectures that are designed for highly reproducible and efficient cell loading process for performing assays on single effector cells such as ASCs. The device structures provided herein: (i) enable facile and efficient loading of analysis chambers with cells or particles, (ii) enable the immobilization of cells and/or particles while preserving the ability to exchange medium components, (iii) are compatible with high resolution microscopy, (iv) provide reduced background fluorescent signal from soluble fluorescent reagents, (v) provide a high-density assay format, (vi) facilitates recovery of selected cells or particles from any given chamber, and (vii) can be integrated with screening instrumentation.

Devices provided herein are designed to accommodate tens of thousands to millions of cells per use. The number of effector cells isolated per chamber, and per device run is a function of the concentration of cells in a cell suspension loaded onto a device, the frequency in the cell suspension of the specific effector cell(s) being selected for, and the total number of chambers on a device. Devices with arrays up to and greater than 1,000,000 extracellular effect assay chambers can be fabricated and employed.

The devices provided herein are microfluidic in that each comprises features (e.g., assay chambers and/or fluidic channels) that include a minimum dimension of less than 1 mm. However, in embodiments provided herein, the minimum dimension of a feature of one of the devices is <500 μm, <100 μm, or <30 μm.

The microfluidic devices presented herein each comprises a plurality of assay chambers. The assay chambers may be present in a single component of a dual component microfluidic device, or present in a single component device. When multiple components of a device are separated from one another, and/or when a single component microfluidic device is employed, the assay chambers are open to the environment, i.e., the chambers can be accessed directly from above, e.g., via pipetting or pouring, and therefore, are referred to herein as “open chambers”. It will be understood that although the chambers are initially open, once a second “top” component is placed above, on top of the chambers, the chambers can be considered closed.

Assay chambers of a device provided herein in one embodiment have an average minimum lateral dimension of from about 50 μm to about 300 μm, from about 50 μm to about 250 μm, from about 50 μm to about 200 μm, from about 50 μm to about 150 μm, from about 50 μm to about 100 μm. In another embodiment, assay chambers of a device have an average minimum lateral dimension of about 50 μm, about 75 μm, about 100 μm, about 125 μm, 150 μm or about 200 μm.

Assay chambers of a device provided herein in one embodiment have an average height (depth) of from about 50 μm to about 300 μm, from about 50 μm to about 250 μm, from about 50 μm to about 200 μm, from about 50 μm to about 150 μm, from about 50 μm to about 100 μm. In another embodiment, assay chambers of a device have an average height/depth of about 50 μm, about 75 μm, about 100 μm, about 125 μm, 150 μm or about 200 μm.

The assay chambers of a device, in one embodiment, have an average volume of from about 100 pL to about 100 nL, or from about 100 pL to about 10 nL, or from about 100 pL to about 1 nL. In one embodiment, the average volume of the assay chambers of the device is about 100 pL, about 200 pL, about 300 pL, about 400 pL, about 500 pL, about 600 pL, about 700 pL, about 800 pL, about 900 pL or about 1 nL. In another embodiment, the volume of the assay chamber is about 2 nL, from about 1 nL to about 5 nL, from about 1 nL to about 4 nL or from about 1 nL to about 3 nL. In another embodiment, the average volume of the assay chambers of a device is from about 10 pL to about 10 nL, from about 10 pL to about 1 nL, from about 10 pL to about 100 pL, from about 500 pL to about 5 nL, from about 50 pL to about 5 nL, from about 1 nL to about 5 nL, or from about 1 nL to about 10 nL. In another embodiment, the average volume of the assay chambers in a device is about 150 pL, about 250 pL, about 350 pL, about 450 pL, about 550 pL, about 650 pL, about 750 pL, about 1 nL, about 5 nL, or about 10 nL.

The devices provided herein, in one embodiment, harness a gravity-based immobilization of cells and/or particles. Gravity based immobilization allows for perfusion of non-adherent cell types, and in general, buffer and reagent exchange within, through, or over the chambers containing effector cells and/or readout particles, e.g., over the chamber floors. In one device embodiment, each chamber has a cubic geometry with an access channel passing over the top. The access chamber can be formed in a top component of a device and aligned to a bottom component comprising open assay chambers, as described herein. Alternatively, the access channel can be formed by a microfabricated trench in the bottom component of the device which connects with the open chamber structures.

During loading of the device chambers, in one embodiment, particles (e.g., cells or beads) are introduced directly over open chambers, located in a bottom component of a two-component device and fall to the bottom of the chambers. In single component device embodiments, the single component is the component that includes the open chambers and therefore is the component that is loaded with particles. In an embodiment where a multicomponent device is employed, cells may be introduced through access channels formed at the interface of the top component and bottom component of the device. In this case, cells follow streamlines and pass over the chambers during loading, but then fall to the bottom of the chambers when the flow is stopped. Due to the laminar flow profile, the flow velocity is negligible near the chamber bottom. This is true when perfusing the combined device by flowing a solution through channels. This is also true within the chambers when exchanging the liquid over the open chambers in the bottom component when gently pipetting or aspirating liquid, e.g., when a fluid reservoir is provided overtop the chambers. This fluidic architecture allows for perfusion of the chamber array, and the exchange of reagents via combined convection/diffusion, without disturbing the location of non-adherent cells (or readout particles such as readout beads) in the chambers.

In one embodiment, the component that comprises the assay chambers comprises a structure to allow for a medium reservoir directly overtop the chambers. The medium reservoir is provided in one embodiment, to block evaporation, to provide nutrients or other molecules to the chambers, to ensure cell viability and/or to provide growth medium for cells. The reservoir structures can be provided over the entire chamber array, or a portion thereof. In another embodiment, multiple reservoir structures are provided in different sections of the chamber array, to allow for delivery of different medium, e.g., cell growth medium or assay reagents, to different portions of the array. In one embodiment, the reservoir structure is fabricated to allow for from about 1 mL to about 20 mL, from about 2 mL to about 20 mL, from about 3 mL to about 20 mL or from about 4 mL to about 20 mL of medium to be provided overtop chambers. In another embodiment, the reservoir structure is fabricated to allow for from about 1 mL to about 15 mL, or from about 2 mL to about 15 mL, or from about 2 mL to about 15 mL, or from about 3 mL to about 15 mL of medium to be provided overtop chambers. Where multiple reservoir structures are utilized on a single device, such structures can be fabricated to allow for a reservoir volumes of from about 100 μL to about 2 mL per individual reservoir. In one embodiment, where a reservoir structure is provided, medium exchange is carried out by aspirating from the reservoir and then providing a new medium overtop the array into the reservoir.

The device architectures provided herein are designed so that the soluble secretion products of effector cells (or a portion of the secretion products) are not washed away from a chamber when additional components, e.g., accessory particles or cell signaling ligands, are added to the chamber, or when a perfusing or wash liquid/medium is introduced. Additionally, the devices provided herein allow for the addition of components to a chamber without the introduction of substantial cross-contamination of secretion products (e.g., antibodies) between individual chambers of a device. Such isolation of chambers from one another is achieved in part by fabricating chambers with aspect ratios (i.e., the height/depth to the minimum lateral dimension) of ≥about 0.6, or ≥about 1. Therefore, the devices provided herein each comprise a plurality of chambers such that the individual chambers of the plurality each have a depth greater than or equal to about 60% of the minimum lateral dimension of the chamber. In a further embodiment, the aspect ratio of the individual chambers (i.e., the height/depth to the minimum lateral dimension) is ≥about 1.1, ≥about 1.5, ≥about 2, ≥about 2.1≥about 2.5, ≥about 3, ≥about 4 or ≥about 5. In another embodiment, the average aspect ratio of chambers in a device is ≥about 0.6, ≥about 0.7, ≥about 0.8, ≥about 0.9, ≥about 1, ≥about 1.1, ≥about 1.5, ≥about 2, ≥about 2.1≥about 2.5, ≥about 3, ≥about 4 or ≥about 5. In another embodiment, the average aspect ratio of chambers in a device is ≥0.6, but ≤15, ≥0.8, but ≤10, ≥1, but ≤10, ≥1.1, but ≤10, ≥1.2, but ≤10, ≥1, but ≤5, or ≥1.5, but ≤10.

Devices provided herein, or portions thereof, are fabricated via soft lithography. In some embodiments, a device comprises a separate top and bottom component, and at least one of the components is fabricated via soft lithography. The devices are transparent or substantially transparent to facilitate imaging of assay chambers. In some embodiments, the top and bottom components of the device are elastomeric, for example, the top and bottom components, or the single component, is fabricated from polydimexylsiloxane (PDMS).

The devices provided herein include a component that comprises an array of assay chambers. The chamber dimensions are in one embodiment, defined in photoresist, and the chambers are then cast from a polymeric material such as PDMS via a soft lithographic process. Soft lithography processes and the use of positive and negative photoresists in photolithography are known to those of ordinary skill in the art, see for example, Xia and Whitesides (1998). Agnew. Chem. Int. Ed. Engl. 37(5), pp. 551-575, the contents of which are incorporated by reference in their entirety for all purposes. It is intended that a chamber cross-section is not constrained to any particular shape, and that chambers may be fabricated via soft lithography having a cross-section that is circular, oval, rectangular, triangular, or other shape.

In some embodiments, an imprinting process is used in which a liquid elastomer such as PDMS is poured onto a microfabricated substrate such as a silicon wafer patterned with a thick photoresist, the poured PDMS is then degassed, a glass substrate is placed on top of the microfabricated substrate, pressed down to extrude excess polymer material, heated to polymerize the PDMS, and then separated to leave a thin molded PDMS layer on the glass substrate. In one embodiment, the thickness of the PDMS layer between the bottom of molded chambers and the glass substrate is less than approximately 5 to 50 μm. In one embodiment, the total thickness of material below the molded chambers is determined primarily by the thickness of the glass substrate and may be less than approximately 100 μm, 200 μm, 400 μm, or 1 mm. Devices with arrays of high aspect ratio chambers, e.g., ≥about 0.6 or ≥about 1, or from about 1 to about 10, molded directly onto thin transparent substrates allows for high resolution imaging through the bottom of the glass substrate. Devices having arrays of chambers with aspect ratios provided herein may also be created by other microfabrication processes known in the art including hot embossing, injection molding, reactive ion etching, or other anisotropic etching processes.

In some embodiments, a device provided herein includes a “top component” having multiple layers, for example, to define microfluidic channels that include valves to seal the channels. In a further embodiment, the “bottom component” of the device includes open chambers which as provided above can be fabricated via soft lithography. The open chambers can be sealed or substantially sealed (with small channels or opening connecting adjacent chambers) upon the introduction of the top component immediately above the bottom component, where a face of each component is in contact.

A top component of a device that includes multiple layers can be fabricated via multilayer soft lithography (MSL) (Unger et al. (2000). Science 7, pp. 113-116, incorporated by reference in its entirety). Moreover, where both a “top component” and “bottom component” are fabricated from an elastomeric material, for example, via soft lithography, and are brought together to form a non-reversible seal, the multiple components of the device can be characterized as being fabricated by MSL.

Amongst all microfluidics technologies, MSL is unique in its rapid and inexpensive prototyping of devices having thousands of integrated microvalves (Thorsen et el. (2002). Science 298, pp. 58-584). These valves can be used to build higher-level fluidic components including mixers, peristaltic pumps (Unger et al. (2000). Science 7, pp. 113-116) and fluidic multiplexing structures (Thorsen et al. (2002). Science 298, pp. 58-584; Hansen and Quake (2003). Curr. Opin. Struc. Biol. 13, pp. 538-544) thus enabling high levels of integration and on-chip liquid handling (Hansen et al. (2004). Proc. Natl. Acad. Sci. U.S.A. 101, pp. 14431-1436; Maerkl and Quake (2007). Science 315, pp. 233-237). The disclosures of each of the foregoing references in this paragraph are incorporated by reference herein in their entireties for all purposes.

The MSL fabrication process takes advantage of well-established photolithography techniques and advances in microelectronic fabrication technology. The first step in MSL is to draw a design of flow and control channels using computer drafting software, which is then printed on high-resolution masks. Silicon (Si) wafers covered in photoresist are exposed to ultraviolet light, which is filtered out in certain regions by the mask. Depending on whether the photoresist is negative or positive, either areas exposed (negative) or not (positive) crosslinks and the resist will polymerize. The unpolymerized resist is soluble in a developer solution and is subsequently washed away. By combining different photoresists and spin coating at different speeds, silicon wafers are patterned with a variety of different shapes and heights, defining various channels and chambers. The wafers are then used as molds to transfer the patterns to polydimethylsiloxane (PDMS). In one embodiment, prior to molding with PDMS and after defining photoresist layers, molds are parylene coated (chemical vapor deposited poly(p-xylylene) polymers barrier) to reduce sticking of PDMS during molding, enhance mold durability and enable replication of small features.

In MSL, stacking different layers of PDMS cast from different molds on top of each other is used to create channels in overlapping layers. The two (or more) layers are bound together by mixing a potting prepolymer component and a hardener component at complementary stoichiometric ratios to achieve vulcanization.

In one embodiment, fluid flow in the device is controlled using off-chip computer programmable solenoids which actuate the pressure applied to fluid in channels of a top component, e.g., to direct reagent flow over chambers in a bottom component.

As will be appreciated by one of skill in the art, the thickness of a photoresist layer can be controlled in part by the speed of spin coating and the photoresist selected for use. The bulk of the assay chambers, in one embodiment, are defined by an SU-8 100 feature which sits directly on the Si wafer. As known to those of skill in the art, SU-8 is a commonly used epoxy-based negative photoresist. Alternatively, other photoresists known to those of skill in the art can be used to define assay chambers with the heights described above. In some embodiments, the assay chambers have a height and width of 50-500 μM and 50-500 μM, respectively, as defined by the SU-8 features.

The soft lithography and MSL fabrication techniques allow for a wide range of device densities, and chamber volumes to be fabricated. For the devices provided herein, in one embodiment, from about 2000 to about 1,000,000 analysis chambers, e.g., from about 1000 to about 200,000 analysis chambers are provided in a single device, i.e., in the bottom component of the device. The effector cell analysis chambers, in one embodiment, have an average volume of from about 0.5 nL to about 4 nL, for example, from about 1 nL to about 3 nL, or from about 2 nL to about 4 nL.

Devices provided herein allow for the long-term culture and maintenance of cells (e.g., from about 12 hours to about 2 weeks, or from about 12 hours to about 10 days, or from about 12 hours to 5 days, or from about 12 hours to 2 days, or for about a day), whether effector, accessory or of the readout variety. In one embodiment, arrays of chambers in a bottom component can be overlaid with a reservoir of cell growth medium. In another embodiment, a top component comprising a thick membrane (e.g., from about 150 μm to about 500 μm thick, about 200 μm thick, about 300 μm thick, about 400 μm thick or about 500 μm thick) of PDMS elastomer. The membrane is positioned over the chambers and in one embodiment, a reservoir of medium, for example 1 mL of medium is overlaid over the membrane. A similar format has been described previously (Lecault et al. (2011). Nature Methods 8, pp. 581-586, incorporated by reference herein in its entirety for all purposes). The proximity of the medium reservoir (osmotic bath) to the chambers effectively blocks evaporation (through the gas-permeable PDMS material) and ensures robust cell viability and where cells are not fully differentiated, growth over several days, and is conducive for achieving long-term culture in nL volumes with growth rates and cellular responses that are identical to μL volume formats.

In some embodiments, the assembly of the two-component device creates rows of assay chambers in the bottom component that are connected by fluidic channels (top component) that pass over the top of the chambers. The chambers are designed with a sufficiently high aspect ratio that when fluid is provided over the chambers, the velocity of the flow profiles at the bottom of the chambers is greatly attenuated or impeded. During loading of a cell population, or a bead population, a suspension of cells or beads is loaded directly into the bottom component (or the single component) of the device that features the array of open chambers. This can be achieved by delivering, e.g., via pipetting, a volume of cell suspension with a known number of cells onto the top of the open chamber array in the bottom component. Once a sufficient number of cells/beads has been introduced to the array, the cells/beads, which are denser than the surrounding liquid, fall to the bottom of the chambers. Once at the bottom of the chambers, the cells, are effectively sequestered from the flow since the flow velocity is negligible at the bottom of the chamber. In various embodiments, the device provided herein includes active microvalves that allow for channels in a top component that connect to the chambers in a bottom component to be closed, thereby isolating each chamber. Such valve structures may also be used to deliver reagents to specific regions of the array in a controlled and automated fashion. When flow is again passed over top of the chambers it is sufficiently attenuated at the bottom of the chamber such that the cells/beads do not move. However, the short length scale between the top of the chamber and the bottom of the chamber is such that diffusion can quickly exchange medium contents within the chambers of the array. In this way, the device allows for the immobilization of suspension cell types and beads while preserving the ability to perform wash steps needed to refresh or exchange medium contents. Another feature of this device embodiment and device geometry is that the chambers are fabricated such that the bottom of the chambers is in close proximity to a glass substrate and allows for high-quality imaging using an inverted microscope configuration or other instrumentation.

Chamber isolation from its surrounding environment is achieved in one embodiment, without physically sealing the chamber from its surrounding environment. Rather, in one embodiment, isolation is achieved by limiting the fluid communication between chambers to preclude significant contamination between one chamber and adjacent chambers. For example, instead of using one or more valves, adjacent chambers are fabricated with an aspect ratio to limit diffusion between chambers, and/or are separated using an immiscible fluid phase, such as an oil, to block chamber inlets and/or outlets. For example, chambers are designed such that the diffusion of molecules in and out of chambers is sufficiently slow that it does not significantly impede the analysis of secreted products (e.g., antibodies) within a chamber. For example, without wishing to be bound by theory, if chambers are designed such that the total minimum distance that a molecule must diffuse to move from the bottom of one chamber to the bottom of another is more than 2 times the minimum lateral dimension of a chamber, then the ratio of the secreted molecules that interact with a readout particle in the originating chamber to the ratio of secreted molecules that interact with a readout particle in an adjacent chamber is more than 10, provided that interaction with readout particles causes the secreted molecule to bind to the readout particle. This condition is satisfied if the aspect ratio of the chamber, defined as the height to the minimum lateral dimension, is ≥0.6, e.g., ≥1, ≥0.6, but ≤10, ≥1, but ≤10, ≥1.25, but ≤5. Notably, this aspect ratio is also sufficient to allow for exchange of reagents in the chambers while retaining cells in the chambers.

Device embodiments provided herein greatly facilitate cell and reagent handling, as well as cell recovery from chambers in the respective arrays by loading reagents and cells directly into or out of open chambers.

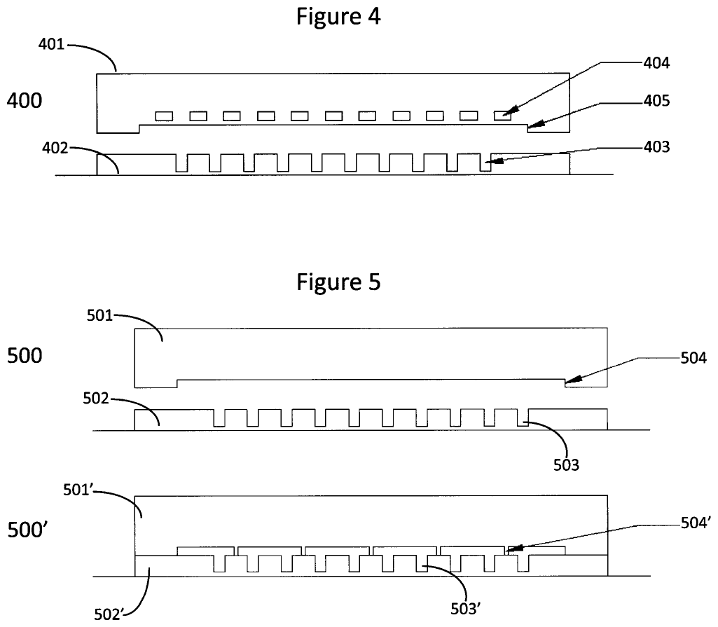

In one embodiment, a “split” microfluidic device is provided for performing one or more of the extracellular effect assays described herein. The split device includes two components, a top component and a bottom component. The bottom component includes the extracellular effect assay chambers. The top component, as described herein, can also be multilayered and fabricated via MSL. In another embodiment, the top component is a single layer. One embodiment of the split device is depicted in FIG. 4. FIG. 4 shows two-component device 400 that includes a top component 401 and bottom component 402. The bottom component includes open chambers 403. The top component includes push down valve structures 404 and an open channel 405.

Because wash and perfusion steps are implemented in the extracellular effect assays described herein, can be performed at relatively low pressures, and are not typically adversely affected by some leaking between adjacent chambers, this split device architecture can be used for carrying out the methods described herein. Further, the existence of a diffusion path between chambers does not result in significant contamination between chambers, provided that the total distance of the diffusion path is sufficiently long compared to the diffusion path between a secreting cell and the readout particles in the chamber having the secreting cells, or the diffusion path is constricted so as to limit diffusive mass transport between adjacent chambers, or both. Thus, adjacent chambers do not require a tight seal. As such, a device comprising two separate components is provided for carrying out one or more of the extracellular effect assays described herein: (i) the bottom component comprising an open array of high aspect ratio chambers and (ii) a top fluidic component that includes open microchannels on the bottom layer that are designed to align to the array of chambers of the bottom component, and integrated microvalves that may be used to control the flow through these microchannels, but not necessary to seal chambers from the surrounding environment (FIG. 4). Such multilayers in the top component are fabricated for example via MSL, described above. The top component is aligned and pressed to the bottom component such that the microvalves are positioned between chambers and thus when actuated, seal off respective chambers from one another. It is also intended that the channel structures may be defined by the bottom component of the device and that the top component may be used to define valves that operate on these channels by deflecting a membrane that impedes flow down the channels.

This split microfluidic device greatly facilitates cell loading as compared to a non-split device, i.e., a device fabricated via MSL that includes fully sealed layers, i.e., and chambers required to be addressed via microchannels. The open chamber array of the bottom component is first loaded by addition of a cell suspension and/or particles. Once the cells/particles have settled into the chambers of the split device, the top component of the device is positioned over the array to execute the fluid handling and imaging steps required for screening. Once an assay is complete, the top component of the device may be again removed to expose the array in the bottom component and recover the cells, or the readout particles, or both, from the selected chambers. In addition to simplifying the loading procedure this strategy also simplifies the fabrication of devices since it is not necessary to fabricate devices within a thin PDMS membrane. Notably, the use of chambers having aspect ratios of ≥0.6, e.g., ≥0.7, ≥1, is critical to this approach since it preserves the ability to exchange solutions without washing the cells out of chambers, and because it allows for the removal of the top substrate without creating flow within the chambers that would cause the cells to be lost. In one embodiment, the aspect ratio of the bottom component of the device comprising the open chambers is ≥0.6, e.g., ≥0.7, ≥1, but less than about 10.

In another embodiment, a “split” two-component microfluidic device is provided for performing one or more of the extracellular effect assays described herein. The split device includes two components, a top component and a bottom component. The top component comprises a single layer and the bottom component comprises a single layer. Each layer can be fabricated via soft lithography as described herein. It should also be noted that although a “two-component” device includes two components, these components are different from multiple “layers” that are present in a single component device fabricated by MSL. The two-component devices described herein are formed via reversible bonds and can be separated after individual assay steps, whereas layers of a device fabricated by MSL cannot be separated without device failure.

Cross sections of schematics of various embodiments 500 and 500′ of the split device are depicted in FIG. 5 (top and bottom). In these embodiments, unlike the split device with a multilayer top, the top component of device 500 does not include integrated valves and instead consists of a single layer 501 comprising a channel structure or flow cell structure 504 (FIG. 5). Although this version of the device does not include active valves, it can be used in conjunction with peripheral control fluidics to control the flow and exchange of reagents across the chamber array. In another embodiment, the top component has a featureless surface and is used to form channels defined in the bottom component.

A variety of flow structures can be used in the top component 501 and 501′, e.g., a single wide flow cell 504 (FIG. 5, top), a flow cell with a collection of “stand-off” spacers 504′ (FIG. 5, bottom), an array of channels, etc. It is further noted that because cell loading into chambers 503 and 503′ of the bottom component 502 and 502′ is performed prior to assembly of the full device, the flow structures in a top or bottom component may be more narrow than in a conventional microfluidic device, e.g., a microfluidic device fabricated by MSL. For example, flow structures can have minimum dimensions of from about 2 μm, or 5 μm, 10 μm, or 20 μm.

Although this geometry does not include microvalves to seal assay chambers from one another, the use of small channels that connect one or more chambers to an inlet and an outlet through which solution can be flowed is sufficient to slow diffusion between adjacent chambers and thereby reduce cross-contamination to levels that do not impede assay performance. It should further be noted that in addition to minimizing diffusive transport between chambers, the use of channels with small cross-sections, such as the channels described herein, results in high fluidic impedance that suppresses any unwanted convection between chambers during incubation. Further, the top component may be designed such that the pressure used to bring the top and bottom components of the device together is used to modulate the cross-section of the flow path connecting the chambers and even to seal the chambers. For instance, the top component may be fabricated with compliant “stand-off” structures 504′ that can be collapsed by increasing the pressure, thereby allowing for the array to be sealed without the need of a deflectable membrane structure between the two components (FIG. 5, bottom). It should be noted that although FIG. 5 depicts the “stand-off structure” between every other chamber, the structure can be employed between every chamber, every third chamber, etc. In addition to facilitating loading and preserving the key functionality of the device set forth in WO 2014/153651, the disclosure of which is incorporated by reference herein in its entirety, this embodiment also greatly simplifies device fabrication and is readily amenable to automation in an instrument by having a series of macroscopic valves and fluidics used to control delivery to the array.

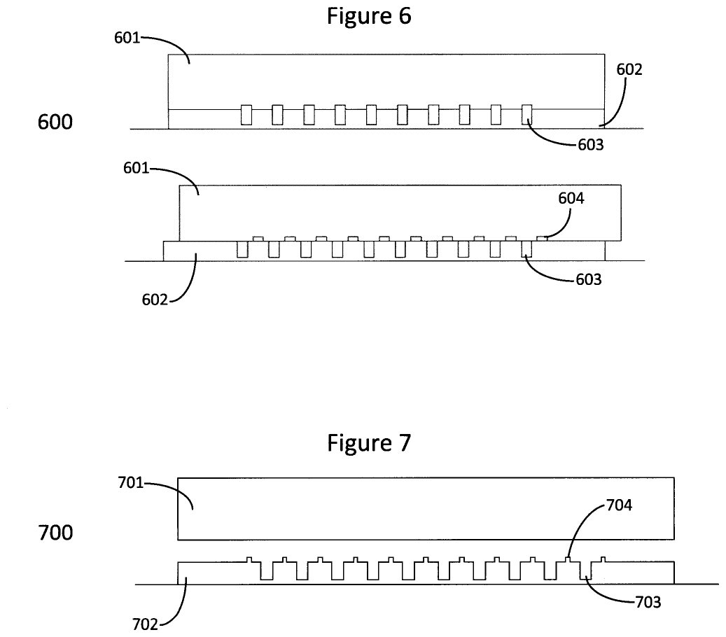

In yet another embodiment, a split device comprising (i) a bottom substrate component comprising an open array of high aspect ratio chambers of ≥0.6, e.g., ≥0.7, ≥1, but <about 10, ≥1, but <about 5, and (ii) a top component designed to allow for reversible sealing of chambers within the array of the bottom component by translation of the top component relative to the bottom component. A cross sectional schematic of such as a device 600 comprising a top component 601 and bottom component 602 comprising microchambers 603 is provided in FIG. 6. In this embodiment, the top component 601 of device 600 is fabricated with a conduit of microchannels 604 (FIG. 6) which when aligned over the bottom component of the device and chambers 603, provides a fluidic path for the delivery of solutions to the chambers 603 of the array. After fluidic/reagent delivery, the top component can be translated relative to the bottom component (FIG. 6, bottom). Upon doing so, the channels 604 are brought out of contact with the chambers and the chambers 603 are sealed by the surface of the top component (FIG. 6, bottom). Translationally shifting the top layer provides a method to fully seal the chambers in the bottom component. The advantages of cell and reagent handling and cell recovery that are present for other split component devices also exist for this device.

Yet another split component device embodiment includes a device 700 comprising a top component 701 and a bottom component 702, a cross sectional drawing of which is provided at FIG. 7. As shown in FIG. 7, the top component 701 includes an unpatterned top. Specifically, the split component device comprises (i) a bottom component comprising an open array of chambers 703 each having an aspect ratio of ≥0.6, e.g., ≥0.7, or ≥1, but <about 10, ≥1, but <about 5, and (ii) a top component that is substantially flat and unpatterned. The bottom component includes one or more raised sections (spacers) 704 between chambers of the chamber array. In one embodiment, the raised sections are fabricated via soft lithography and photolithography using multiple photoresists, as known to those of ordinary skill in the art. The one or more raised sections define a spacing between the top component and bottom component when the two components are brought together.

In this embodiment, flow across the chambers is less controlled compared to the other split devices described herein, but may still be achieved by generally applying a pressure across the array, for example by hydrostatic pressure created by a liquid column or by creating a flow using a dispensing instrument such as a pipette, or by exchanging the medium overtop of the second piece and then briefly moving the second piece up and down to evoke a fluid transfer to the array. The top component may be brought directly into contact with the surface of the array of chambers to seal one or more of the chambers during incubation.

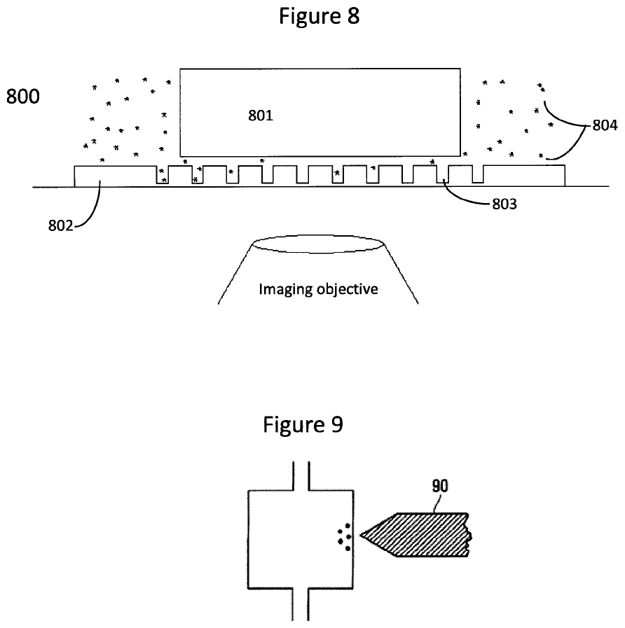

In one embodiment, an open chamber microfluidic array is provided, comprising a plurality of chambers for performing one or more of the extracellular effect assays described herein. In this embodiment, the array is used without a top component when performing the one or more extracellular effect assays. However, a top component, e.g., a top device component fabricated from PDMS, is optionally employed and covers only a portion of the chamber array during imaging (readout of the extracellular effect assay). A schematic of a cross section of such a device 800 is provided at FIG. 8. As with the split devices, the array chambers are fabricated with a depth to ensure that the cells/particles are not disturbed by flow (i.e., the chambers 803 have an aspect ratio of ≥about 0.6, e.g., ≥0.7, ≥1, ≥1 but <about 10, ≥1 but <about 5), but also so that the diffusion path from the bottom of one chamber to another is sufficiently long so that relative efficiency of the capture of cell secretion products (e.g., antibodies) by particles in the chamber that the respective cell is located in, as compared to an adjacent chamber is greater than 5 for a defined experimental condition (e.g. incubation time, number of beads, bead size, chamber spacing, etc.).

Relative Efficiency of capture=RE=# cell secretion products from a chamber with a cell that are captured in the chamber with the cell/# cell secretion products from a chamber with a cell that are captured in an adjacent chamber.

In this embodiment, the unpatterned top component 801, if it is used, eliminates background fluorescence signal that is derived from a large volume of soluble fluorescent molecules 804 that are present over the top of the chamber array during imaging. The size of the unpatterned top 801 defines the volume of medium it is able to displace, for example, medium in a reservoir overlying the chamber array or a portion thereof.

As an example, consider the case where antibodies within a chamber are being captured on antibody capture beads located in the same chamber. Once captured, the antibodies are exposed to a soluble fluorescent antigen that may be added to the solution over top of the array, or added by first removing a portion or substantially all the solution covering the array followed by applying a solution with the soluble fluorescent antigen. If the antibody binds to the fluorescent antigen, the antibody bound on the beads will accumulate fluorescent signal. However, there will also be a large volume of fluorescent antigen above the beads and this will create background fluorescence that will obscure the specific fluorescent signal. If the fluorescent molecules are removed prior to imaging this will result in a low concentration of antigen in the bulk solution, causing the amount of bound antibody on the bead to go down at a speed dictated by the off-rate of the antibody-antigen interaction. Even for a reasonably strong interaction (˜1 nM), this may cause a significant degradation in the signal within a time frame shorter than the imaging time of the array (e.g., about 10 min). However, this background fluorescence can be eliminated, without washing the array, by using a top component 801 to displace the fluorescent solution from the vicinity of the array during the image acquisition step. This top component may for instance be fixed to the imaging system and positioned over the image acquisition region while the device is translated during the capturing of images for the whole array.

In some embodiments, effector cells and readout particles are distributed within a chamber by coating one or more walls of the chamber, e.g., via surface functionalization. In one embodiment, the surface functionalization is performed by graft, covalently linking, adsorbing or otherwise attaching one or more molecules to the surface of the chamber, or modifying the surface of the chamber, such that the adherence of cells or particles to the chamber surface is altered. Nonexclusive examples of such functionalizations for use herein are the non-specific adsorption of proteins, the chemical coupling of proteins, the non-specific adsorption of polymers, the electrostatic adsorption of polymers, the chemical coupling of small molecules, the chemical coupling of nucleic acids, the oxidization of surfaces, etc. PDMS surface functionalization has been described previously, and these methods can be used herein to functionalize surfaces of the devices provided herein (see, e.g., Zhou et al. (2010). Electrophoresis 31, pp. 2-16, incorporated by reference herein for all purposes). Surface functionalizations described herein, in one embodiment, selectively bind one type of effector cell (e.g., an effector cell present in a cell population), or selectively bind one type of readout particle. In another embodiment, the surface functionalization is used to sequester all readout particles present in a chamber.

In yet another embodiment, a surface coating, which can be a surface functionalization, or a plurality of different surface functionalizations are spatially defined within a chamber of a device. Alternatively, a surface functionalization covers the entire chamber surface. Both embodiments are useful for the distribution of effector cells are readout particles into distinct locations within an assay chamber. For instance, in embodiments where the entire chamber is functionalized with a molecule that binds all types of introduced readout particles, the particles are immobilized on the functionalized (coated) surface. In another embodiment, the entire chamber is functionalized to bind only one specific type of readout particle, where multiple readout particle species are introduced. In this embodiment, all particles, are first directed to one region using one of the methods described herein, causing a subset of particles to adhere to the chamber surface in the functionalized region, followed by exerting a force towards a different region that displaces only the particles that do not bind the functionalized surface or surfaces. In yet another embodiment, regions of the device (e.g., different chambers or regions within a single chamber) are functionalized with different molecules that selectively bind different subsets of effector cells and/or readout particles, such that inducing the interaction of the effector cells and/or readout particles with substantially the entire chamber surface results in the partitioning of different particle or cell types in different regions. As described herein, it is intended that surface functionalization may be used in isolation or in combination with the other methods described herein for effector cell and readout particle manipulation. Multiple combinations of particle and cell sequestration methods, together with multiple fluidic geometries are possible.

In another embodiment, effector cells and/or readout particles are positioned within a chamber using a magnetic field. It will be understood that to manipulate and position an effector cell(s) and/or a readout cell(s) magnetically, the effector cell(s) and/or a readout cell(s) are first functionalized with or exposed to magnetic particles that bind to them. In one embodiment, the magnetic field is externally created, i.e., by using a magnet outside of the microfluidic device, using a permanent magnetic, an electromagnet, a solenoid coil, or of other means. In another embodiment, referring to FIG. 9, the magnetic field is generated locally by a magnetic structure 90 integrated into, or separate from, the device. The magnetic field, in one embodiment is applied at different times, and in one embodiment, the magnetic field is applied in conjunction with particle loading, to influence the position of effector cells and/or readout particles that respond to a magnetic field. Commercially available beads or nanoparticles that are used in the separation and or purification of biological samples can be used in the devices and methods provided herein. For example, “Dynabeads” (Life Technologies) are superparamagnetic, monosized and spherical polymer particles, and in one embodiment, are used as readout particles. Magnetic particles conjugated with molecules that specifically bind different target epitopes or cell types are well-known in the art, and are also amenable for use with the devices and methods provided herein. When in the presence of a magnetic field having a non-uniform property, such magnetic particles are subjected to a force that is directed towards the gradient of the magnetic field. This gradient force, in one embodiment, is applied to position particles within a chamber.

Once a chamber is loaded with a cell population and a readout particle or readout particle population, and optionally additional reagent(s) for carrying out an assay on the cell population, the chamber, in one embodiment, is fluidically isolated from one or more remaining chambers of the microfluidic device.

As provided herein, in one aspect, the present invention relates to a method of identifying a cell population comprising an effector cell displaying an extracellular effect. Once it is determined that the cell population demonstrates the extracellular effect, the cell population or portion thereof is recovered to obtain a recovered cell population. Recovery, in one embodiment, comprises accessing a chamber of one of the devices herein with a microcapillary or micropipette and aspirating the chamber's contents or a portion thereof to obtain a recovered cell population. Because the devices provided herein employ chambers in a bottom component that is reversibly sealable with a top component, once a chamber with a cell of interest is identified, the contents of which are easily recovered via aspiration or pipetting via separating the two components and directly accessing the chamber(s).

The recovered cell population(s), in one embodiment, are subjected to further analysis, for example to identify a single effector cell or a subpopulation of effector cells from the recovered cell population that is responsible for the extracellular effect. The recovered cell population(s) can be analyzed in limiting dilution, as subpopulations for a second extracellular effect, which can be the same or different from the first extracellular effect. Cell subpopulations displaying the second extracellular effect can then be recovered for further analysis, for example for a third extracellular effect on a device of the invention, or by a benchtop method, for example RT-PCR and/or next generation sequencing. Alternatively, the nucleic acid from the cell population is sequenced, e.g., to determine the antibody sequences of the population. The antibody can then be cloned and expressed for further testing.

Recovery of one or more cells from one or more assay chambers, in one embodiment, comprises magnetic isolation/recovery. For example, in one embodiment, an assay chamber is exposed to a magnetic particle (or plurality of magnetic particles) that adheres to the one or more cells within the chamber. Adherence can be either selective for a single cell, a subpopulation of the population of cells in the well(s), or non-selective, i.e., the magnet can adhere to all cells. In this case, cells labeled with magnetic particles are drawn to a magnetic probe that creates a magnetic field gradient. The probe, in one embodiment, is designed to enable the magnetic field to be turned on and off, causing cells to adhere to it for removal and then be released during deposition. (EasySep Selection Kit, StemCell Technologies).

Single cells or a plurality of cells harvested from chambers, in one embodiment, are deposited into one or more receptacles for further analysis, for example, open micro-wells, micro-droplets, tubes, culture dishes, plates, petri dishes, enzyme-linked immunosorbent spot (ELISPOT) plates, a second microfluidic device, the same microfluidic device (in a different region), etc. The choice of receptacle is determined by one of skill in the art, and is based on the nature of the downstream analysis and/or storage.

In some embodiments, cell-derived products or intracellular materials are recovered from assay chambers of interest, alternatively or in addition to the recovery of a single cell or plurality cells from identified chambers. For example, if an assay chamber is identified as having a cell that demonstrates a variation in an extracellular effect, in one embodiment, the secretion products from the chamber are is recovered for downstream analysis (e.g., sequence analysis). In another embodiment, the cell or plurality of cells is lysed on the microfluidic device, e.g., within the chamber that the first assay is performed, and the lysate is recovered, e.g., for further analysis such as nucleic acid sequencing.

In another embodiment, the cells in all chambers or a subset of chambers are lysed using a lysis reagent, and then the contents of a given chamber or subset of chambers are recovered. In another embodiment, the cells within a chamber of interest or chambers of interest are lysed in the presence of beads that capture the RNA released from the cells followed by recovery of the beads. In this case the RNA may also be converted to cDNA using a reverse transcriptase enzyme prior to or subsequent recovery.

Following the recovery of cells or cell-derived materials from a chamber or chambers of interest, these materials or cells are analyzed to identify or characterize the isolate or the single cell or plurality of cells. Further analysis can be via one of the devices provided herein (see, e.g., FIG. 2), one of the devices described previously, e.g., in WO 2014/153651, or via conventional benchtop methods. Accordingly, the present invention allows for multiple rounds of microfluidic analysis, for example to identify a cell subpopulation from a recovered cell population that displays a second extracellular effect, a third extracellular effect and or a fourth extracellular effect. By repeating the extracellular effect assays on recovered cell populations, the user of the method obtains highly enriched cell populations for a functional feature of interest, or multiple functional features of interest. Alternatively, a second extracellular effect assay is not carried out and the cell population demonstrating the extracellular effect is recovered and subjected to nucleic acid sequencing, e.g., to determine the sequences of the antibodies in the population.

In one embodiment, one or more cell populations exhibiting the extracellular effect (e.g., a variation in an extracellular effect as compared to another population or a control value), are recovered to obtain one or more recovered cell populations. The one or more individual cell populations are further analyzed to determine the cell or cells responsible for the observed extracellular effect, e.g., at limiting dilution as cell subpopulations. In one embodiment, the method comprises retaining a plurality of cell subpopulations originating from the one or more recovered cell populations in separate chambers of a microfluidic device as described herein. Each of the separate chambers comprises a readout particle population comprising one or more readout particles. The individual cell subpopulations are incubated with the readout particle population. The individual cell subpopulations are assayed for a second extracellular effect, wherein the readout particle population or subpopulation thereof provides a readout of the second extracellular effect. The second extracellular effect can be the same extracellular effect or a different extracellular effect as the extracellular effect measured on the recovered cell population. Based on the second extracellular effect assay, one or more individual cell subpopulations are identified that exhibit the second extracellular effect (e.g., a variation in the second extracellular effect as compared to another population or a control value). The one or more individual cell subpopulations in one embodiment, are then recovered for further analysis, e.g., nucleic acid sequencing.

The term “cell sub-subpopulation” is meant to refer to a subpopulation of a cell subpopulation. In one embodiment, cells from a recovered subpopulation or plurality of cell subpopulations are retained in a plurality of vessels as cell sub-subpopulations. One of skill in the art will recognize that a cell subpopulation can be partitioned into further subpopulations, and the use of the term “sub-subpopulation” is not necessary to make this distinction. Rather, the methods provided herein allow for the further analysis of a recovered cell population at limiting dilution. Each cell subpopulation is present in an individual vessel. The individual subpopulation or sub-subpopulation is lysed, and one or more nucleic acids within each lysed cell subpopulation or lysed cell sub-subpopulation are amplified. In a further embodiment, the one or more nucleic acids comprise an antibody gene.

Several approaches including analysis with one of the devices provided herein may be used for this downstream analysis, depending on the nature of the cells, the number of cells in the original screen, and the intent of the analysis. In one embodiment, where an effector cell population is recovered from a chamber, or a plurality of populations are recovered from multiple chambers, each cell of the plurality is isolated into an individual vessel (e.g. individual assay chamber) and analysis is performed on each effector cell individually. Alternatively, the contents of the multiple chambers can be pooled into an individual chamber or vessel for downstream analysis such as nucleic acid sequencing, e.g., to determine the antibody sequences in the population. Bioinformatics can be used to pair heavy and light chains, if necessary.

In another embodiment, where a population of effector cells is recovered from a chamber, or a plurality of populations are recovered from multiple chambers, the cell populations are reintroduced onto the same microfluidic device in a separate region, or into a second device, and the cells are isolated in chambers at a limiting dilution, i.e., as cell subpopulations, e.g., the cells are isolated at a density of about a single cell per chamber, or from about 2 to about 10 cells per chamber and a second extracellular effect assay is performed. The downstream analysis may be on any size cell subpopulations, for example, the same size as the initial extracellular effect cell assay, e.g., when cells from multiple chambers are pooled, or a smaller population size, e.g., a single cell, two cells, from about 2 cells to about 20 cells, from about 2 cells to about 25 cells. Readout particles are introduced into the chambers comprising the cell subpopulations, and the second extracellular effect assay is performed. The contents of chambers that comprise a cell subpopulation displaying the extracellular effect (e.g., a variation in an extracellular effect as compared to another cell population or a control value), are recovered for further analysis. This further analysis can be with one of the devices provided herein (e.g., by performing a third extracellular effect assay, single cell PCR), benchtop analysis (e.g., PCR, next generation sequencing) or a combination thereof.

In one embodiment, individual recovered effector cells are expanded in culture by distributing the plurality of cells at limiting dilution into a plurality of cell culture vessels to obtain clones from the recovered cells. For example, in an embodiment where a plurality of effector cells of a cell line engineered to express a library of antibodies are present in a chamber or chambers of interest, the cells from the chamber or chambers are subjected to limiting dilution to isolate single effector cells that were present in the chamber or chambers. The single effector cells are then used to obtain clonal populations of each respective effector cell. One or more of the clonal populations can then be analyzed to assess which effector cell produces the antibody of interest by measuring the properties of the secreted antibodies (e.g., by ELISA or a functional assay).

Alternatively, or additionally, cells are recovered from an assay chamber, isolated, e.g., by limiting dilution, and expanded to obtain sufficient material for the sequencing or amplification and purification of one or more genes of interest, e.g., a gene that encodes an antibody. In yet another embodiment, cells are recovered from an assay chamber, isolated, e.g., by limiting dilution, and used for single-cell DNA or mRNA amplification, e.g., by the polymerase chain reaction (PCR) or reverse transcriptase (RT)-PCR, followed by sequencing, to determine the sequence of one or more genes of interest. In another embodiment, a population of cells that exhibits an extracellular effect is recovered from one or more assay chambers, isolated, e.g., by limiting dilution, and subsequently used for single-cell DNA or mRNA amplification of the genes of interest, followed by the cloning of these genes into another cell type for subsequent expression and analysis. In another embodiment, nucleic acid from a population of cells is sequenced in the same reaction.

In one embodiment, the recovered cell population(s) or subpopulation(s) may be isolated and used for an in vivo analysis, for example by injecting them (or antibodies from the recovered cell population) into an animal, or expanded in culture.

In one embodiment, a cell population or subpopulation is recovered that displays the extracellular effect (e.g., after a first or second extracellular effect assay in one of the devices provided herein) and one or more nucleic acids of the recovered cells are subjected to amplification. Amplification, in one embodiment, is carried out by the polymerase chain reaction (PCR), 5′-Rapid amplification of cDNA ends (RACE), in vitro transcription or whole transcriptome amplification (WTA). In a further embodiment, amplification is carried out by reverse transcription (RT)-PCR. RT-PCR can be on single cells, or a plurality of cells of the population. Two main approaches for recovery of antibody genes from single cells include RT-PCR using degenerate primers and 5′ rapid amplification of cDNA ends (RACE) PCR. In one embodiment, the RT-PCR method is based on gene-specific template-switching RT, followed by semi-nested PCR and next-generation amplicon sequencing. Nucleic acid analysis can also be carried out on pooled cell populations, followed by a bioinformatic approach to identify heavy and light chain antibody pairs.

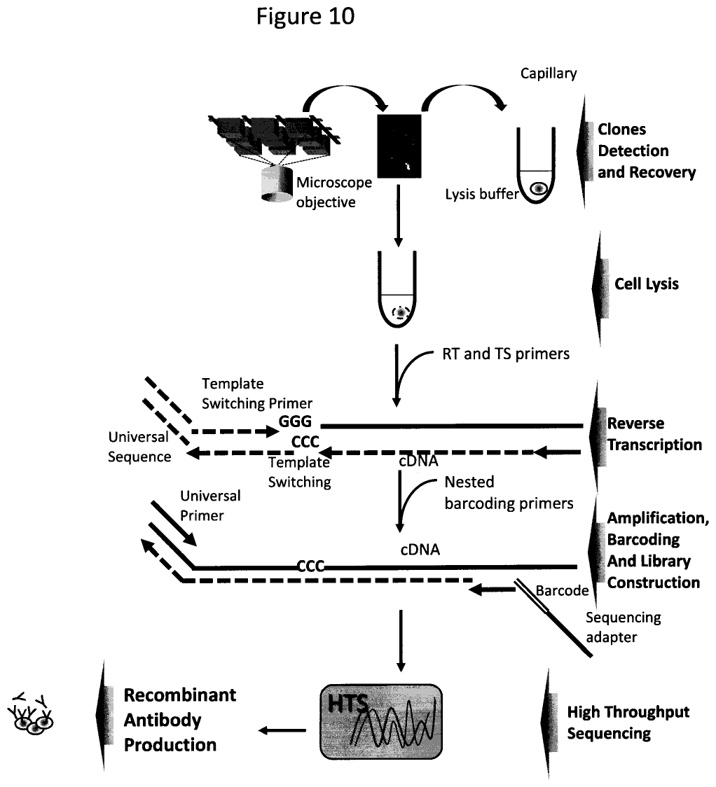

One embodiment of an RT-PCR method for use with identified effector cells displaying a variation in an extracellular effect is shown at FIG. 10. The schematic shows a single cell HV/LV approach using template switching reverse transcription and multiplexed primers. In this embodiment, single cells are deposited into microfuge tubes and cDNA is generated from multiplexed gene-specific primers targeting the constant region of heavy and light chains. Template-switching activity of MMLV enzyme is used to append the reverse complement of a template-switching oligo onto the 3′ end of the resulting cDNA (Huber et al. (1989). J. Biol. Chem. 264, pp. 4669-4678; Luo and Taylor (1990). J. Virology 64, pp. 4321-4328; each incorporated by reference in their entireties for all purposes). Semi-nested PCR (common 3′ primers and multiplexed nested primers positioned inside the RT primer region) using multiplexed primers at constant region of heavy and light chain, and a universal primer complementary to the copied template switching oligo, is used to amplify cDNA and introduce indexing sequences that are specific to each single cell amplicon. The resulting single cell amplicons are pooled and sequenced.

In some cases, a recovered cell population, following recovery from the microfluidic device, is not further isolated into single cells, or subjected to limiting dilution for further analysis. For example, if the plurality of cells isolated from a chamber contain a cell that secretes an antibody of interest (e.g., present in a population of one or more additional cells that secrete other antibody(ies)), the plurality of cells, in one embodiment, is expanded in culture to generate clonal populations of the cells of the plurality, one or some of which make the desired product (i.e., the antibody of interest). In another embodiment, a plurality of cells recovered from a chamber is lysed (either on the microfluidic device or after recovery), followed by amplification of the pooled nucleic acid population from the lysate, and subjected to analysis by sequencing. In this case, a bioinformatics analysis of the sequences obtained may be used to infer, possibly using information from other sources, which of the sequences is likely to encode for the protein of interest (for example, an antibody). Importantly, the analysis method afforded by the present invention is greatly simplified, as compared to bulk analysis of a large numbers of cells, due to the limited number of cells that are recovered. This limited number of cells provides a reduced complexity of the genomic information within the population of cells.

In one embodiment, the amplified DNA sequences of the plurality of cells are used to create libraries of sequences that are recombinantly expressed in an immortalized cell line, per methods known to those of skill in the art. This cell line may then be analyzed, possibly by first isolating clones, to identify the antibody genes of interest. In some instances, these libraries are used to screen for combinations of genes that result in a protein complex of interest. For example, in one embodiment, genes include both heavy and light chains of antibody genes to identify full length antibody sequences. The complexity of such analyses is greatly reduced by the fact that the number of cells from a recovered chamber is small. For example, if there are 10 cells in the chamber that displays the extracellular effect, there are only 100 possible antibody heavy and light chain pairings. By comparison, bulk samples typically have thousands of different antibody sequences, corresponding to millions of possible pairings.

In some embodiments, recovered cells may contain different cell types that can be further isolated using methods known to those of skill in the art. For instance, if the assay chamber comprises both ASCs and fibroblasts, the latter used to maintain the ASCs, the ASCs in one embodiment, are separated from the fibroblasts after recovery, e.g., by using affinity capture methods.

In one embodiment, an assay is carried out on a plurality of cell populations, present in individual chambers of a single device, to determine whether an effector cell within one or more of the populations secretes an antibody or other biomolecule that inhibits the growth of a cell or exhibits some other extracellular effect. The chamber's contents can then be recovered for further analysis, for example, at limiting dilution of the effector cells to determine which effector cell is responsible for the effect. Antibody sequences can also be recovered and sequenced by methods known to those of skill in the art. As described herein, the devices of the invention greatly facilitate cell handling and cell recovery.

After identifying a cell population(s) that contain one or more effector cells displaying the extracellular effect of interest, the cell population(s), in one embodiment, is analyzed again, but at limiting dilution, e.g., as single cells in individual chambers (or other reaction vessel), or smaller populations in individual chambers (as compared to the first screen), to determine the identity of the individual effector cell(s) responsible for the extracellular effect. One embodiment of this two-step screening method is shown in FIG. 2. Once the effector cell(s) responsible for the extracellular effect is identified, its genetic information can be amplified and sequenced.

In one aspect, the devices and methods provided herein allow for identification of a cell population displaying an extracellular effect compared to one or more other cell populations. That is, a comparison of the extracellular effect signal is made with a control value or the value demonstrated by some other chamber or plurality of chambers in the device. In this aspect, individual cell populations are retained in separate chambers of a device, wherein at least one of the individual cell populations comprises one or more effector cells and the separate chambers each comprise a readout particle population, each comprising one or more readout particles. The cell populations are assayed for the presence of the extracellular effect, whereby the readout particle population or subpopulation thereof provides a readout of the extracellular effect. A cell population from amongst the plurality can then be identified that exhibits the extracellular effect, for example, a different effect signal as compared to one or more of the remaining cell populations of the plurality, or a control value. Once a cell population is identified that displays the extracellular effect, the population is recovered and may be further assayed at limiting dilution to identify the cell or cells within the population responsible for the extracellular effect.

Cell populations that can be analyzed herein are not limited to a specific type or source. For example, in one embodiment a population of cells partitioned into individual cell populations in chambers may be peripheral blood mononuclear cells (PBMCs) isolated from an animal that has been immunized or exposed to an antigen. The population of cells in another embodiment are B-cells isolated from an animal that has been immunized or exposed to an antigen. The source of the population of cells may be whole blood from the animal that has been immunized or exposed to an antigen. The population of cells may be from lymphoid tissues or bone marrow or spleen from the animal that has been immunized or exposed to an antigen. In cases where it is desirable to look at a naïve repertoire the source of the population of cells may be from an animal that has not been immunized or exposed to an antigen.

Individual cell populations optionally comprising one or more effector cells are assayed to determine whether the respective cell populations comprise an effector cell that exhibits an extracellular effect (e.g., a variation in an extracellular effect as compared to another cell population or a control value). Additionally, a cell population comprising an effector cell need not include multiple effector cells, or be a population of only effector cells. Rather, non-effector cells, in embodiments described herein, are included in the population. The non-effector cells can be a majority or minority of the population. A heterogeneous population comprising an effector cell need not include multiple effector cells. Rather, a heterogeneous cell population is heterogeneous if two cells are heterogeneous with respect to one another. A cell population in a device chamber can comprise zero effector cells, one effector cell or a plurality of effector cells. Similarly, a cell subpopulation can comprise zero effector cells, one effector cell or a plurality of effector cells.

The extracellular effect in one embodiment is a binding property (e.g., kinetic measurement, specificity, affinity, etc.), or some other extracellular effect of the cell or a biomolecule secreted by the cell, e.g., an antibody. For example, in one embodiment, the extracellular effect is agonism or antagonism of a cell surface receptor, agonism or antagonism of an ion channel or agonism or antagonism of an ABC transporter, modulation of apoptosis, modulation of cell proliferation, a change in a morphological appearance of a readout particle, a change in localization of a protein within a readout particle, expression of a protein by a readout particle, neutralization of the biological activity of an accessory particle, cell lysis of a readout cell induced by an effector cell, cell apoptosis of a readout cell induced by the effector cell, cell necrosis of the readout cell, internalization of an antibody by a readout cell, internalization of an accessory particle by a readout cell, enzyme neutralization by the effector cell, neutralization of a soluble signaling molecule, or a combination thereof.

The presence and identification of an effector cell that secretes a biomolecule (e.g., antibody) that binds a target of interest (e.g., antigen) is readily ascertained in embodiments where the effector cell is present in a heterogeneous cell population comprising a plurality of effector cells that secrete antibodies that are not specific to the target of interest. In one embodiment, this is achieved in an individual device chamber by first capturing in the chamber, all or substantially all of the secreted antibodies of the population on a readout particle(s) (e.g., bead) functionalized to capture antibodies (for example, functionalized with protein G or protein A), addition of fluorescently labeled antigen into the chamber and imaging of the particle(s) to detect the presence or absence of an increase in fluorescence due to binding of the antigen to immobilized antibody(ies). An estimate of minimum number of antibodies captured on a bead that is required for reliable detection may be obtained by performing experiments to measure antibody secretion from single cells. In one embodiment, it is possible to detect antigen-specific antibodies secreted from a single ASC in a heterogeneous population of approximately 250 cells. A cell population present in an individual reaction chamber can therefore comprise from about two to about 250 cells, e.g., from about 10 to about 100 cells per chamber, or from about 10 to about 50 cells per chamber. In another embodiment, a chamber comprises about 2 to about 250 ASCs, or from about 2 to about 100 ASCs. In one embodiment, greater than 80% of the chambers of a device contain a cell population. As stated above, a cell population can contain cells other than effector cells and not all cell populations will contain an effector cell. This is particularly true when a conventional enrichment protocol (e.g., FACS) is not able to be used to obtain a substantially pure cell population of the same cell type.

In one embodiment, where the imaging of individual cells or readout particles is required, the number of cells in a population is chosen to be insufficient to cover the floor of the chamber being imaged, so that the cells being imaged are arranged in a monolayer. Alternatively, the cell population includes a number of cells that is insufficient to form a bilayer covering a surface of the chamber.

In some embodiments, larger populations of cells can be present in a population within a single chamber, without inhibiting the detection of an extracellular effect that stems from a single effector cell or a small number of effector cells in the population. For example, in one embodiment, the number of cells in a cell population is from 2 to about 300, or from about 10 to about 300, or from about 100 to about 300. In another embodiment, the number of cells in a cell population is from 2 to about 250, or from about 10 to about 250, or from about 100 to about 250. In another embodiment, the number of cells in a cell population is from 2 to about 200, or from about 10 to about 200, or from about 100 to about 200. In another embodiment, the number of cells in a cell population is from 2 to about 100, or from about 10 to about 100, for from about 50 to about 100. In another embodiment, the number of cells in a cell population is from 2 to about 90, or from about 10 to about 90, or from about 50 to about 900. In yet another embodiment, the number of cells in a cell population is from 2 to about 80, or from 10 to about 80, or from 2 to about 70, or from about 10 to about 70, or from about 2 to about 60, or from about 10 to about 60, or from about 2 to about 50, or from about 10 to about 50, or from about 2 to about 40, or from about 10 to about 40, or from 2 to about 30, or from about 10 to about 20, or from 2 to about 10. In some embodiments, the majority of cells in a cell population are effector cells.

A cell sample, in one embodiment, is separated into a plurality of cell populations in thousands of chambers, and individual cell populations within single chambers are assayed for an extracellular effect. One or more individual cell populations are identified and recovered if an effector cell within the one or more populations exhibits the extracellular effect. The extracellular effect is determined by the user and in one embodiment, is a binding interaction with an antigen, cell surface receptor, ABC transporter or an ion channel.

Although the methods provided herein can be used to identify a single effector cell (alone or within a heterogeneous population) based on a binding interaction, e.g., antigen affinity and specificity, the invention is not limited thereto. Rather, identification of a cell population, in one embodiment, is carried out via the implementation of a direct functional assay. The present invention therefore includes methods and devices that enable the direct discovery of an ASC within a cell population that secretes a “functional antibody,” without the need to initially screen the “functional antibody” for binding properties such as affinity and selectivity to an antigen target.

Along these lines, functional antibodies and receptors discoverable by the methods herein are provided. For example, the nucleic acid of an effector cell responsible for an extracellular effect is amplified and sequenced. The nucleic acid is a gene encoding for a secreted biomolecule (e.g., antibody, or fragment thereof), or a gene encoding a cell receptor or fragment thereof, for example a T-cell receptor. The antibody or fragment thereof or cell receptor or fragment thereof can be cloned and/or sequenced by methods known in the art. For example, in one embodiment, an ASC that secretes a functional antibody is one that modulates cell signaling by binding to a targeted cell surface protein, such as an ion-channel receptor, ABC transporter, a G-protein coupled receptor (GPCR), a tyrosine kinase receptor (RTK) or a receptor with intrinsic enzymatic activity such as intrinsic guanylate cyclase activity.

A cell population comprising one or a plurality of effector cells is identified in a chamber, based on the result of an extracellular effect assay carried out in the chamber. If an extracellular effect is attributable to the cells in the chamber, the cell population is recovered and analyzed to determine the effector cell or effector cells within the population responsible for the effect (see, e.g., FIG. 2). In embodiments where the effector cell secretes antibodies, the DNA sequence that encodes the antibody produced by the ASC or ASCs can then be determined and subsequently cloned. In one embodiment, the antibody DNA sequences are cloned and expressed in cell lines to provide an immortal source of monoclonal antibody for further validation and pre-clinical testing.

In one embodiment, a cell population comprises a population of cells genetically engineered to express libraries of molecules that may bind a target epitope, cells genetically engineered to express genes or fragments of genes derived from cDNA libraries of interest, cells genetically engineered with reporters for various biological functions, and cells derived from immortalized lines or primary sources. Clones originating from a single cell, in one embodiment, are heterogeneous with respect to one another due to for example, gene silencing, differentiation, altered gene expression, changes in morphology, etc. Additionally, cells derived from immortalized lines or primary sources are not identical clones of a single cell and are considered heterogeneous with respect to one another. Cells derived from a single cell, but which naturally undergo somatic hypermutation or are engineered to undergo somatic hypermutation (e.g., by inducing expression of activation-induced cytidine deaminase, etc.), are not considered clones and therefore these cells, when present together, are considered a heterogeneous cell population.

Once a cell population(s) is identified as demonstrating the extracellular effect, in one embodiment, it is selectively recovered to obtain a recovered cell population. If multiple cell populations are identified as exhibiting the extracellular effect, in one embodiment, the multiple cell populations are recovered and pooled, to obtain a recovered cell population. The recovered cell population is enriched for effector cells, compared to the starting population of cells originally loaded onto the device in that the former has a larger percentage of effector cells as compared to the latter. Alternatively, nucleic acid from cells is sequenced to determine antibody nucleic acid sequences.

In one embodiment, subpopulations of the recovered cell population are assayed for the presence of a second extracellular effect. The second extracellular effect can be the same effect that was assayed on the identified cell population, or a different extracellular effect. In a further embodiment, the subpopulations of the identified population each comprise from about 1 to about 10 cells. In even a further embodiment, the subpopulations of the identified population comprise an average of 1 cell each. One or more of the subpopulations displaying the extracellular effect are then identified and recovered to obtain a recovered subpopulation, which in one embodiment, is enriched for effector cells. If multiple cell subpopulations are identified, in one embodiment, they are recovered and pooled, to obtain a recovered cell subpopulation. The genetic information, e.g., antibody gene sequences or fragments thereof, from the recovered cell subpopulation can then be isolated, amplified and/or sequenced.

The cell populations for use with the invention are not limited by source, rather, they may be derived from any animal, including human or other mammal, or alternatively, from in vitro tissue culture. Cells may be analyzed directly, for example, analyzed directly after harvesting from a source, or after enrichment of a population having a desired property (such as the secretion of antibodies that bind a specific antigen) by use of various protocols that are known in the art, e.g., flow cytometry. Prior to harvesting from an animal source, in one embodiment, the animal is subject to one or more immunizations. In one embodiment, flow cytometry is used to enrich for effector cells prior to loading onto one of the devices provided herein, and the flow cytometry is fluorescence activated cell sorting (FACS). Where a cell population is used that has been enriched for effector cells, e.g., ASCs, and retained as individual cell populations in individual assay chambers, the individual cell populations need not be comprised entirely of effector cells. Rather, other cell types may be present as a majority or minority. Additionally, one or more of the individual cell populations may contain zero effector cells.

There are several methods for the enrichment of ASCs derived from animals known to those of skill in the art, which can be used to enrich and provide a population of cells for analysis by the methods and devices provided herein. For example, in one embodiment, FACS is used to enrich for human ASCs using surface markers CD19⁺CD20^{low}CD27^{hi}CD38^{hi} (Smith et al. (2009). Nature Protocols 4, pp. 372-384). In another embodiment, a cell population is enriched by magnetic immunocapture based positive or negative selection of cells displaying surface markers. In another embodiment, a plaque assay (Jerne et al. (1963). Science 140, p. 405), ELISPOT assay (Czerkinsky et al. (1983). J. Immunol. Methods 65, pp. 109-121), droplet assay (Powel et al. (1990). Bio/Technology 8, pp. 333-337), cell surface fluorescent-linked immunosorbent assay (Yoshimoto et al. (2013), Scientific Reports 3, 1191) or a cell surface affinity matrix assay (Manz et al. (1995). Proc. Natl. Acad. Sci. U.S.A. 92, pp. 1921-1925) is used to enrich for ASCs prior to performing one of the methods provided herein or prior to loading a starting cell population onto one of the devices provided herein. The disclosures of each of the references cited in this paragraph are incorporated by reference herein in their entireties for all purposes.

With respect to the devices provided herein, it is noted that not all chambers on the device necessarily include a cell population and/or a readout particle population, e.g., empty chambers or partially filled chambers may be present.

In some embodiments, it is desirable to have one or more accessory particles, which can include one or more accessory cells present in the assay chambers, to support the viability and/or function of one or more cells in the cell populations or to implement an extracellular effect assay. For example, in one embodiment an accessory cell, or plurality of accessory cells, comprises a fibroblast cell, natural killer (NK) cell, killer T-cell, antigen presenting cell, dendritic cell, recombinant cell, or a combination thereof.

An accessory particle or cell, or a population comprising the same, in one embodiment, is delivered to a chamber together with the cell population and/or with a readout particle population. In one embodiment, an accessory cell is part of a cell population delivered to a chamber. Alternatively, or additionally, the accessory particle(s) or accessory cell(s) is delivered to a chamber prior to, or after, the loading of a cell population being assayed for the extracellular effect, to the chamber or plurality of chambers. Accessory particles (e.g., cells) can be delivered to chambers serially after the cell population is loaded. Delivery can occur through microchannels in a top component of the device, or via directly loading open chambers.

“Accessory particle” as referred to herein means any particle, including but not limited to a protein, protein fragment, or cell, that (i) supports the viability and/or function of an effector cell, (ii) facilitates an extracellular effect, (iii) facilitates the measurement of an extracellular effect, or (iv) detection of an extracellular effect of an effector cell. Accessory particles can therefore refer to soluble molecules.

Accessory particles include but are not limited to proteins, peptides, growth factors, cytokines, neurotransmitters, lipids, phospholipids, carbohydrates, metabolites, signaling molecules, amino acids, monoamines, glycoproteins, hormones, virus particles or a combination thereof. In one embodiment, one or more accessory particles comprises sphingosine-1-phosphate, lysophosphatidic acid or a combination thereof. In some embodiments, accessory particles include proteins, protein fragments, peptides, growth factors, cytokines, neurotransmitters (e.g., neuromodulators or neuropeptides), lipids, phospholipids, amino acids, monoamines, glycoproteins, hormones, virus particles, or a complement pathway activator, upon binding of an effector cell secretion product to a readout cell or a combination thereof. In one embodiment, one or more accessory particles are accessory molecules such as sphingosine-1-phosphate, lysophosphatidic acid or a combination thereof.

As an example of an accessory cell, in one embodiment, a population of fibroblast cells (that do not secrete antibodies) is included within a cell population enriched for effector cells (e.g., ASCs) to enhance the viability of the effector cell(s) (e.g., ASC(s)) within the population. In another embodiment, a population of NK cells may be added as accessory particles to implement an antibody-dependent cell-mediated cytotoxicity assay, where the NK cells will attack and lyse the target cells upon binding of an antibody on their surface. In embodiments where functional cellular assays are carried out on one or more cell populations, it will be appreciated that the effector cell(s) within the one or more cell populations will need to stay viable for an extended period of time while within a chamber of the microfluidic device. To this end, accessory particles and/or accessory cells, in one embodiment, are used to sustain the viability of the cell population that optionally comprises one or more effector cells. As described herein, accessory particles, e.g., accessory cells, can be used to sustain or enhance the viability of a readout cell or population thereof.

One advantage of embodiments described herein is that the analysis of more than one effector cell within a single assay chamber, and/or the analysis of single or a few effector cells in the presence of other cells, allows for much greater assay throughput and hence the identification and selection of desired effector cells that would otherwise be too rare to detect efficiently. This is advantageous where there are limited methods to enrich for a desired cell type or where such enrichment has deleterious effects such as the reduction of viability of the cells being assayed.

ASCs may be identified and isolated without the need for enrichment based on surface markers. In B-cells isolated from PBMCs following immunization, the frequency of ASCs may be between 0.01% and 1%. At a throughput of 10,000,000 to 100,000,000 cells per device run, as may be achieved by loading an array of 1,000,000 chambers with approximately 10 to 100 cells per chamber, it is possible to screen many thousands of ASCs without any prior purification. This is important even in cases where markers are available, because FACS purification of ASCs can reduce cell viability. Additionally, because appropriate reagents for the enrichment of ASCs may not be available for a host species of interest, the ability to screen ASCs without further purification is an advance in the study of ASCs. Following immunization, the frequency of antibody secreting cells in PBMCs may be between 0.01 and 1% and is thus detectable by using the devices provided herein. Thus, since the isolation of peripheral blood mononuclear cells (PBMCs) may be performed on any species without specific capture reagents, some of the present methods provide for the rapid and economical selection of cells secreting antibodies of interest from any species.

ASCs from basal levels in humans, in one embodiment, are identified by the methods and devices provided herein. While animals can be immunized to generate new antibodies against most antigens, the same procedure cannot be performed widely in humans except for approved vaccines. However, humans that have been naturally exposed to an antigen, or vaccinated at some point in their lifespan, typically possess low basal levels of antibody-secreting cells specific for the antigen. The present invention can be used to identify and isolate extremely rare effector cells secreting specific antibodies from a large number of cells (e.g., greater than 100,000 to 100,000,000 per device run). Such methods are used herein for the discovery of functional antibodies, e.g., as therapeutics for autoimmune diseases and cancers where autoantibodies may be present.

As provided throughout, the present invention relates in part to extracellular effect assays carried out in a massively parallel fashion in chambers of a single device. The assays are carried out to measure and detect an extracellular effect exerted by an effector cell, or plurality thereof, present in a cell population. A population of readout particles or subpopulation thereof provides a readout of the extracellular effect. For example, the methods described herein allow for the identification of a heterogeneous cell population which contains an effector cell that exerts the extracellular effect, e.g., secretion of an antibody specific to a desired antigen, in a background of up to about 250 cells (e.g., from about 2 to about 100 cells, or from about 2 to about 50 cells) that do not exert the extracellular effect.

In one embodiment, a cell population subjected to the methods described herein comprises an ASC or a plurality of ASCs, and the readout particle population or subpopulation thereof displays a target epitope or a plurality of target epitopes. The readout particle population in one embodiment is a population of beads functionalized to capture antibodies by an epitope or epitopes. Alternatively, or additionally, the readout particle population is specific for an antibody's Fc region, and therefore, does not discriminate between antibodies having different epitopes. The readout particle population or subpopulation thereof, in one embodiment is labeled with a fluorescently-conjugated molecule containing the target epitope, for example to perform an ELISA assay. Fluorescent based antibody and cytokine bead assays are known in the art, see, e.g., Singhal et al. (2010). Anal. Chem. 82, pp. 8671-8679, Luminex® Assays (Life Technologies), BD™ Cytometric Bead Array, the disclosures of which are incorporated by reference in their entireties. These methods can be used to determine whether an effector cell demonstrates an extracellular effect on the readout particle.

Moreover, as described herein, individual chambers of a device are structured so that reagent exchange within the chambers is possible, whereby cross-contamination is eliminated or substantially eliminated between chambers. This allows for cell culture in individual chambers, as well as the detection of multiple extracellular effects in a single chamber, for example, unique antigen binding events and/or other extracellular effects in a single chamber, for example, by exchanging antigens and secondary antibodies to label the respective binding complexes, followed by imaging. In these serial assay steps, the assays can be carried out with the same fluorophores, as each reaction is performed serially after a wash step. Alternatively, different fluorophores can be used to detect different extracellular effects in a serial manner, or in parallel, in one assay chamber. For example, chambers are fabricated with an aspect ratio (defined as the height to the minimum lateral dimension) of ≥1, to achieve reagent exchange without substantial cross contamination. In one embodiment, the average aspect ratio of the plurality of chambers of the device is ≥about 0.6, about ≥0.7, about ≥0.8, about ≥0.9, ≥about 1, ≥about 1.5, ≥about 2, ≥about 2.5, ≥about 3, ≥about 3.5, ≥about 4, ≥about 4.5, ≥about 5, ≥about 5.5, ≥about 6. In yet another embodiment, the average aspect ratio of the plurality of chambers of the device is ≥about 1, but <10, or ≥about 1, but <about 9, or ≥about 1, but <about 8.

In one embodiment, the readout particle population is a readout cell population wherein at least some of the readout cells display a target epitope on their surfaces. In one embodiment, the readout cell population, or a subpopulation thereof, is alive and viable. In another embodiment, the readout cell population or a subpopulation thereof is fixed. As will be recognized from the discussion above, where antibody binding is assayed for, “antibody binding” is considered the extracellular effect of an effector cell or plurality of effector cells. Antibody binding can be detected by, for example, staining of the cell with one or more fluorescently labeled secondary antibodies. In another embodiment, binding of an antibody to the target epitope on a readout particle or readout cell causes the death of a readout cell, or some other readout cell response as discussed herein (e.g., secretion of biomolecule, activation or inhibition of a cell signaling pathway).

Readout cells may be distinguished by features e.g., morphology, size, surface adherence, motility and fluorescent response. For example, in one embodiment, a population of readout cells is labeled on their surfaces, or intracellulary, to determine whether the readout cells exhibit a response. For example, calcein, carboxyfluorescein succinymyl ester reporter (CFSE), or GFP/YFP/RFP reporters can be used to label one or more reporter cells, including extracellular receptors and intracellular proteins and other biomolecules.

In some embodiments, the readout particle population is a heterogeneous readout particle population, e.g., a heterogeneous readout cell population. Where, for example, an ASC or plurality of ASCs are present in a cell population, the individual readout particles in the population may display different target epitopes, or display two different cell receptors (e.g., a GPCR or RTK or ion channel or a combination thereof, including multiple species of different classes such as two or more GPCRs, etc.). Accordingly, the specificity of the extracellular effect, e.g., the specificity of an antibody for a target epitope, or the inhibition of a specific cell surface receptor, can be assessed. In another embodiment, an effector cell within a cell population is an ASC, and the readout particle population comprises a heterogeneous bead population that non-selectively captures all antibodies (e.g., Fc region specific) and a bead population that is specific for a unique target epitope.

In one embodiment, accessory particles are provided to facilitate the readout and/or measurement of an extracellular effect. As described throughout, an extracellular effect includes an effect that is exhibited by an effector cell secretion product (e.g., antibody). For example, in one embodiment, a NK cell is provided as an accessory particle, to facilitate the measurement of lysis of a readout cell. In this embodiment, the extracellular effect includes lysis of a readout cell that binds to a specific epitope or cell receptor, by the NK cell, when an antibody secreted by an effector cell binds to the aforementioned readout cell.

In one embodiment, one or more cytokines is used as an accessory particle. Examples of cytokines that can be used as accessory particles include chemokines, interferons, interleukins, lymphokines, tumor necrosis factors. In some embodiments the accessory molecules are produced by readout cells. In some embodiments, a cytokine is used as an accessory particle and is one or more of the cytokines provided in Table 1, below. In another embodiment, one or more of the following cytokines is used as an accessory particle: interleukin (IL)-1α, IL-1β, IL-1RA, IL18, IL-2, IL-3, IL-4, IL-5, IL-6, IL-7, IL-8, IL-9, IL-10, IL-11, IL-12, IL-13, IL-14, IL-15, IL-16, IL17, IL-18, IL-19, IL-20, granulocyte colony-stimulating factor (G-CSF), granulocyte macrophage-colony stimulating factor (GM-CSF), leukemia inhibitor factor, oncostatin M, interferon (IFN)-α, IFN-β, IFN-γ, CD154, lymphotoxin beta (LTB), tumor necrosis factor (TNF)-α, TNF-β, various isoforms of transforming growth factor (TGF)-β, erythropoietin, megakaryocyte growth and development factor (MGDF), Fms-related tyrosine kinase 3 ligand (Flt-3L), stem cell factor, colony stimulating factor-1 (CSF-1), macrophage stimulating factor, 4-1BB ligand, a proliferation-inducing ligand (APRIL), cluster of differentiation 70 (CD70), cluster of differentiation 153 (CD153), cluster of differentiation 178 (CD17)8, glucocorticoid induced TNF receptor ligand (GITRL), LIGHT (also referred to as TNF ligand superfamily member 14, HVEM ligand, CD258), OX40L (also referred to as CD252 and is the ligand for CD134), TALL-1, TNF related apoptosis inducing ligand (TRAIL), tumor necrosis factor weak inducer of apoptosis (TWEAK), TNF-related activation-induced cytokine (TRANCE) or a combination thereof.

TABLE 1
Representative cytokines and their receptors.

| | | | | | Cytokine | |
|---|---|---|---|---|---|---|
| | | Amino | | Molecular | Receptor(s)(Da) | Receptor |
| Name | Synonym(s) | Acids | Chromosome | Weight | and Form | Location(s) |
| Interleukins | | | | | | |
| IL-1α | hematopoietin-1 | 271 | 2q14 | 30606 | CD121a, | 2q12, |
| | | | | | CDw121b | 2q12-q22 |
| IL-1β | catabolin | 269 | 2q14 | 20747 | CD121a, | 2q12, |
| | | | | | CDw121b | 2q12-q22 |
| IL-1RA | IL-1 receptor | 177 | 2q14.2 | 20055 | CD121a | 2q12 |
| | antagonist | | | | | |
| IL-18 | interferon-γ | 193 | 11q22.2-q22.3 | 22326 | IL-18Rα, β | 2q12 |
| | inducing factor | | | | | |
| Common g chain | | | | | | |
| (CD132) | | | | | | |
| IL-2 | T-cell growth | 153 | 4q26-q27 | 17628 | CD25, 122, 132 | 10p15-p14, |
| | factor | | | | | 22q13.1, |
| | | | | | | Xq13.1 |
| IL-4 | BSF-1 | 153 | 5q31.1 | 17492 | CD124, 213a13, | 16p11.2-12.1, |
| | | | | | 132 | X, Xq13.1 |
| IL-7 | | 177 | 8q12-q13 | 20186 | CD127, 132 | 5p13, Xq13.1 |
| IL-9 | T-cell growth | 144 | 5q31.1 | 15909 | IL-9R, CD132 | Xq28 or |
| | factor P40 | | | | | Yq12, |
| | | | | | | Xq13.1 |
| IL-13 | P600 | 132 | 5q31.1 | 14319 | CD213a1,213a2, | X, Xq13.1-q28, |
| | | | | | CD1243, 132 | 16p11.2-12.1, |
| | | | | | | Xq13.1 |
| IL-15 | | 162 | 4q31 | 18086 | IL-15Ra, CD122, | 10p14-p14, |
| | | | | | 132 | 22q13.1, |
| | | | | | | Xq13.1 |
| Common b chain | | | | | | |
| (CD131) | | | | | | |
| IL-3 | multipotential | 152 | 5q31.1 | 17233 | CD123, CDw131 | Xp22.3 or |
| | CSF, MCGF | | | | | Yp11.3, |
| | | | | | | 22q13.1 |
| IL-5 | BCDF-1 | 134 | 5q31.1 | 15238, | CDw125, 131 | 3p26-p24, |
| | | | | homodimer | | 22q13.1 |
| Also related | | | | | | |
| GM-CSF | CSF-2 | 144 | 5q31.1 | 16295 | CD116, CDw131 | Xp22.32 or |
| | | | | | | Yp11.2, |
| | | | | | | 22q13.1 |
| IL-6-like | | | | | | |
| IL-6 | IPN-β2, BSF-2 | 212 | 7p21 | 23718 | CD126, 130 | 1q21, 5q11 |
| IL-11 | AGIF | 199 | 19q13.3-13.4 | 21429 | IL-11Ra, CD130 | 9p13, 5q11 |
| Also related | | | | | | |
| G-CSF | CSF-3 | 207 | 17q11.2-q12 | 21781 | CD114 | 1p35-p34.3 |
| IL-12 | NK cell | 219/328 | 3p12-p13.2/ | 24844/37169 | CD212 | 19p13.1, |
| | stimulatory | | 5q31.1-q33.1 | heterodimer | | 1p31.2 |
| | factor | | | | | |
| LIF | leukemia | 202 | 22q12.1-q12.2 | 22008 | LIFR, CD130 | 5p13-p12 |
| | inhibitory | | | | | |
| | factor | | | | | |
| OSM | oncostatin M | 252 | 22q12.1-q12.2 | 28484 | OSMR, CD130 | 5p15.2-5p12 |
| IL-10-like | | | | | | |
| IL-10 | CSIF | 178 | 1q31-q32 | 20517, | CDw210 | 11q23 |
| | | | | homodimer | | |
| IL-20 | | 176 | 2q32.2 | 20437 | IL-20Rα, β | ? |
| Others | | | | | | |
| IL-14 | HMW-BCGF | 498 | 1 | 54759 | IL-14R | ? |
| IL-16 | LCF | 631 | 15q24 | 66694, | CD4 | 12pter-p12 |
| | | | | homotetramer | | |
| IL-17 | CTLA-8 | 155 | 2q31 | 17504, | CDw217 | 22q11.1 |
| | | | | homodimer | | |
| Interferons | | | | | | |
| IFN-α | | 189 | 9p22 | 21781 | CD118 | 21q22.11 |
| IFN-β | | 187 | 9p21 | 22294 | CD118 | 21q22.11 |
| IFN-γ | | 166 | 12q14 | 19348, | CDw119 | 6q23-q24 |
| | | | | homodimer | | |
| TNF | | | | | | |
| CD154 | CD40L, TRAP | 261 | Xq26 | 29273, | CD40 | 20q12-q13.2 |
| | | | | homotrimer | | |
| LT-β | | 244 | 6p21.3 | 25390, | LTβR | 12p13 |
| | | | | heterotrimer | | |
| TNF-α | cachectin | 233 | 6p21.3 | 25644, | CD120a, b | 12p13.2, |
| | | | | homotrimer | | 1p36.3-p36.2 |
| TNF-β | LT-α | 205 | 6p21.3 | 22297, | CD120a, b | 12p13.2, |
| | | | | heterotrimer | | 1p36.3-p36.2 |
| 4-1BBL | | 254 | 19p13.3 | 26624, | CDw137 (4-1BB) | 1p36 |
| | | | | trimer? | | |
| APRIL | TALL-2 | 250 | 17p13.1 | 27433, | BCMA, TACI | 16p13.1, |
| | | | | trimer? | | 17p11.2 |
| CD70 | CD27L | 193 | 19p13 | 21146, | CD27 | 12p13 |
| | | | | trimer? | | |
| CD153 | CD30L | 234 | 9q33 | 26017, | CD30 | 1p36 |
| | | | | trimer? | | |
| CD178 | FasL | 281 | 1q23 | 31485, | CD95 (Fas) | 10q24.1 |
| | | | | trimer? | | |
| GITRL | | 177 | 1q23 | 20307, | GITR | 1p36.3 |
| | | | | trimer? | | |
| LIGHT | | 240 | 16p11.2 | 26351, | LTbR, HVEM | 12p13, |
| | | | | trimer? | | 1p36.3-p36.2 |
| OX40L | | 183 | 1q25 | 21050, | OX40 | 1p36 |
| | | | | trimer? | | |
| TALL-1 | | 285 | 13q32-q34 | 31222, | BCMA, TACI | 16p13.1, |
| | | | | trimer? | | 17p11.2 |
| TRAIL | Apo2L | 281 | 3q26 | 32509, | TRAILR1-4 | 8p21 |
| | | | | trimer? | | |
| TWEAK | Apo3L | 249 | 17p13.3 | 27216, | Apo3 | 1p36.2 |
| | | | | trimer? | | |
| TRANCE | OPGL | 317 | 13q14 | 35478, | RANK, OPG | 18q22.1, |
| | | | | trimer? | | 8q24 |
| TGF-β | | | | | | |
| TGF-β1 | TGF-β | 390 | 19q13.1 | 44341, | TGF-βR1 | 9q22 |
| | | | | homodimer | | |
| TGF-β2 | | 414 | 1q41 | 47747, | TGF-βR2 | 3p22 |
| | | | | homodimer | | |
| TGF-β3 | | 412 | 14q24 | 47328, | TGF-βR3 | 1p33-p32 |
| | | | | homodimer | | |
| Miscellaneous | | | | | | |
| hematopoietins | | | | | | |
| Epo | erythropoietin | 193 | 7q21 | 21306 | EpoR | 19p13.3-p13.2 |
| Tpo | MGDF | 353 | 3q26.3-q27 | 37822 | TpoR | 1p34 |
| Flt-3L | | 235 | 19q13.1 | 26416 | Flt-3 | 13q12 |
| SCF | stem cell | 273 | 12q22 | 30898, | CD117 | 4q11-q12 |
| | factor, c-kit | | | homodimer | | |
| | ligand | | | | | |
| M-CSF | CSF-1 | 554 | 1p21-p13 | 60119, | CD115 | 5q33-q35 |
| | | | | homodimer | | |
| MSP | Macrophage | 711 | 3p21 | 80379 | CDw136 | 3p21.3 |
| | stimulating | | | | | |
| | factor, MST-1 | | | | | |

Adapted from Cytokines, Chemokines and Their Receptors. Madame Curie Bioscience Database. (Landes Biosceince)

In one embodiment, an accessory particle is a cytokine or other factor operable to stimulate a response of the readout cell. For example, readout cells may be incubated with an effector cell or plurality thereof and pulsed with a cytokine that is operable to affect the readout cell. Alternatively, or additionally, cytokine-secreting cells operable to affect the readout particles are provided to the chamber as accessory cells. Neutralization of the secreted cytokines by an effector cell secretion product in one embodiment, are detected by the absence of the expected effect of the cytokine on the readout cell. In another embodiment, an accessory particle is provided and is a virus operable to infect one or more readout cells, and neutralization of the virus is detected as the reduced infection of readout cells by the virus.

As described herein, in one embodiment, the extracellular effect discernable with the methods and devices provided herein is a functional effect. The functional effect, in one embodiment is apoptosis, modulation of cell proliferation, a change in a morphological appearance of the readout particle, a change in aggregation of multiple readout particles, a change in localization of a protein within the readout particle, expression of a protein by the readout particle, secretion of a protein by the readout particle, triggering of a cell signaling cascade, readout cell internalization of a molecule secreted by an effector cell, or neutralization of an accessory particle operable to affect the readout particle.

Once an extracellular effect is identified in a chamber comprising a cell population, the population is recovered and a downstream assay can be performed on subpopulations of the recovered cell population, to determine which effector cell(s) is responsible for the measured extracellular effect. Alternatively, the recovered population can be sequenced to determine the antibody sequences of the effector cell(s) in the population. The downstream assay in one embodiment, is carried out on the same device as the first extracellular effect assay. However, in another embodiment, the downstream assay is carried out in a device, for example, a benchtop single cell reverse transcriptase (RT)-PCR reaction. In one embodiment, antibody gene sequences of identified and recovered effector cells are isolated, cloned and expressed to provide novel functional antibodies.

Although functional extracellular effects of single ASCs are measurable by the methods and devices provided herein, affinity, binding and specificity can also be measured as the “extracellular effect” of an effector cell, e.g., an effect of an effector cell secretion product. For example, the binding assay provided by Dierks et al. (2009). Anal. Biochem. 386, pp. 30-35, incorporated by reference herein in its entirety, can be used in the devices provided herein to determine whether an ASC secretes an antibody that binds to a specific target.

In another embodiment, the extracellular effect is affinity or binding kinetics for an antigen, and the method described by Singhal et al. (2010). Anal. Chem. 82, pp. 8671-8679, incorporated by reference herein for all purposes, is used to assay the extracellular effect.

In one embodiment, parallel analyses of multiple extracellular effects are carried out in one chamber by employing multiple types of readout particles. Alternatively, or additionally, parallel analyses of multiple functional effects are carried out on a single microfluidic device by employing different readout particles in at least two different chambers.

The readout particle, in some embodiments, is a molecule, for example an enzyme. In one embodiment, the readout particle is an enzyme that is present as a soluble molecule, or that is tethered to the chamber surface or to another physical support in the chamber. In this case, the binding of an antibody that inhibits the enzymatic activity of the readout particle, in one embodiment, is detected by reduced signal that reports on the enzymatic activity, including a fluorescent signal or colorometric signal or precipitation reaction.

In one embodiment, determining whether an effector cell or multiple effector cells within a cell population display an extracellular effect comprises the use of light and/or fluorescence microscopy of the assay chamber containing the cell population. Accordingly, one embodiment of the invention involves maintaining the readout particle population in a single plane to facilitate imaging of the particles by microscopy. In one embodiment, a readout particle population in a chamber is maintained in a single plane that is imaged through the device material, or portion thereof (e.g., glass or PDMS) to generate one, or many, high-resolution images of the chamber. In one embodiment, a high-resolution image is an image comparable to what is achieved using standard microscopy formats with a comparable optical instrument (lenses and objectives, lighting and contrast mechanisms, etc.).

The cell population and readout particle population can be loaded simultaneously into a chamber (e.g., in different or the same solution by loading directly over the chambers, e.g., by hydrostatic pressure created by a liquid column, by creating a flow using a dispensing instrument such as a pipette, or by exchanging the medium overtop of the bottom component and moving the top component or bottom component up and down to evoke a fluid transfer to the microchambers). Alternatively, the effector cells and readout particles are loaded serially into a chamber. A person of ordinary skill in the art will understand that the cell population can be provided to a chamber prior to (or after) the loading of the readout particle(s) to the chamber. However, it is possible that the readout particle population and cell population be provided together as a mixture. As with accessory particles, readout particles can be loaded directly into the bottom component of a device, i.e., directly into open chambers, or through channels in a top component of a device. As such, device architecture will dictate how loading is carried out.

In embodiments, an individual readout particle population and cell population are retained in single chambers of a device. In one embodiment, the chamber is substantially isolated from other chambers of the device that also comprise individual cell populations and a readout particle population, for example, to minimize contamination between chambers. Isolation can occur with or without fluidic structures. For example, in one embodiment, the top component of a device can include a control channel with a thin membrane below it. When brought together with the bottom layer channels connecting chambers, valving can occur. Alternatively, the top component can be set up in a “push-down” geometry with an open channel structure on the bottom face and control lines passing over top.

However, complete isolation is not necessary to practice the methods provided herein. In one embodiment, where isolation is desired, isolation comprises fluidic isolation, and fluidic isolation of chambers is achieved by physically sealing them, e.g., by using a translatable device (see, e.g., FIG. 6). However, isolation in another embodiment is achieved without physically sealing the chamber, by limiting fluid communication between chambers to preclude contamination between one chamber and another chamber of the device, either by convection or by diffusion. For instance, chamber geometries (e.g., particular aspect ratios) may be selected so as to suppress convective transport between chambers and so that the distance between an effector cell and the readout particles in the same chamber is much less than the distance between the effector cell and the readout particles in any other chamber, thereby ensuring that diffusion to and accumulation of secreted molecules on the readout particles in any given chambers is predominantly from the effector cells in that chamber.

To carry out the extracellular effect assay, a cell population which optionally comprises one or more effector cells, and a readout particle population are incubated together in a chamber, and these incubations are carried out in a massively parallel manner within each device. It will be appreciated that additional incubation step(s) can take place, e.g., when components such as accessory particles are added to a chamber, and that an initial incubation step can occur prior to the addition of readout particles, and/or after readout particles are added to a chamber comprising a cell population.

For example, an incubation step can include a medium exchange to keep the cell population healthy, and/or a cell wash step. Incubation can also comprise addition of accessory particles (e.g., accessory molecules) used to carry out an extracellular effect assay.

An incubating step, in one embodiment, includes controlling one or more of properties of the chamber, e.g., humidity, temperature and/or pH to maintain cell viability (effector cell, accessory cell or readout cell) and/or maintain one or more functional properties of a cell in the chamber, such as secretion, surface marker expression, gene expression, signaling mechanisms, etc. In one embodiment, an incubation step includes flowing a perfusing fluid through or over the chamber (e.g., over a chamber surface) or plurality of chambers. The perfusing fluid is selected depending on type of effector cell and/or readout cell in the chamber. For example, a perfusing liquid in one embodiment, is selected to maintain cell viability, e.g., to replenish depleted oxygen or remove waste productions, or to maintain cellular state, e.g., to replenish essential cytokines, or to assist in assaying the desired effect, e.g., to add fluorescent detection reagents. Perfusion can also be used to exchange reagents, for example, to assay for multiple extracellular effects in a serial manner.

In another embodiment, incubating a cell population includes flowing a perfusing fluid through or over a chamber (e.g., over a chamber surface) or plurality of chambers to induce a cellular response of a readout particle (e.g., readout cell). For example, the incubating step in one embodiment comprises adding a fluid comprising signaling cytokines to a chamber comprising the cell population. The incubating step can be periodic, continuous, or a combination thereof. For example, flowing a perfusing fluid to an assay chamber or chambers is periodic or continuous, or a combination thereof. In one embodiment, the flow of an incubating liquid is pressure driven, for example by using compressed air, syringe pumps or gravity to modulate the flow.

Once individual chambers within a device are provided with a cell population and a readout particle population, a method is carried out to determine whether a cell within the population exhibits an extracellular effect on the readout particle population or subpopulation thereof. The cell population and readout particle population and/or subpopulation thereof, as appropriate, is then examined to determine whether a cell(s) within a population exhibits the extracellular effect. It is not necessary that the specific cell or cells displaying the extracellular effect be identified within the chamber, so long as the presence of the effect is detected within the chamber. In one embodiment, once a cell population is identified as exhibiting an extracellular effect, the cell population is recovered to identify the specific effector cell(s) responsible for the extracellular effect. In another embodiment, once the cell population is identified as exhibiting the extracellular effect (e.g., a variation in an extracellular effect as compared to another cell population or a control value), it is recovered and the nucleic acid from the cell population is amplified and sequenced.

The extracellular effect, in one embodiment, is a binding interaction between the protein (e.g., antibody or fragment thereof) produced by an effector cell(s) and a readout particle, e.g., a bead or a cell. In one embodiment, one or more of the effector cells in the population is an antibody secreting cell (ASC), and the readout particle includes an antigen having a target epitope. The extracellular effect, in one embodiment, is the differential binding to an antigen as compared to a control level or a level exhibited by a second population of cells. Alternatively, the variation in the extracellular effect is the presence of an effector cell that secretes an antibody with a modulated affinity for a particular antigen. That is, the binding interaction is a measure of one or more of antigen-antibody binding specificity, antigen-antibody binding affinity, and antigen-antibody binding kinetics. Alternatively or additionally, the extracellular effect is a modulation of apoptosis, modulation of cell proliferation, a change in a morphological appearance of a readout particle, a change in localization of a protein within a readout particle, expression of a protein by a readout particle, neutralization of the biological activity of an accessory particle, cell lysis of a readout cell induced by the effector cell, cell apoptosis of the readout cell induced by the effector cell, readout cell necrosis, internalization of an antibody, internalization of an accessory particle, enzyme neutralization by the effector cell, neutralization of a soluble signaling molecule or a combination thereof.

Multiple readout particles can be distinguished by one or more characteristics unique to the respective readout particle, such as fluorophore type, varying levels of fluorescence intensity, morphology, size, and surface staining.

Once incubated with a cell population comprising an effector cell(s), the readout particle population or subpopulation thereof is examined to determine whether one or more effector cells within the cell population exhibits an extracellular effect on one or more readout particles, whether direct or indirect (e.g., a variation in an extracellular effect as compared to another cell population or a control value). Cell populations are identified that exhibit the effect, and then are recovered for downstream analysis. As provided throughout, it is not necessary that the specific effector cell(s) displaying the extracellular effect on the one or more readout particles be identified so long as the presence of the extracellular effect is detected within an assay chamber.

In some embodiments, the one or more effector cells secretes biomolecules, e.g., antibodies, and the extracellular effect of the secreted biomolecule is evaluated on a readout particle or a plurality of readout particles (e.g., readout cells) in order to detect a cell population that demonstrates the extracellular effect. In another embodiment, the extracellular effect is an effect of a T-cell receptor, for example, binding to an antigen.

In one embodiment, a readout particle population is a heterogeneous population of readout cells comprising cells engineered to express a cDNA library, whereby the cDNA library encodes for a plurality of cell surface proteins. The binding of antibody to these cells is used to recover cells that secrete antibodies that bind to a target epitope.

In some embodiments, one or more readout particles include a readout cell displaying or expressing a target antigen. In a further embodiment, a natural killer cell, or a plurality thereof is provided to the chamber as an accessory cell(s) that facilitates the extracellular effect (lysis) being measured. The accessory cell can be provided to the chamber with the cell population, the readout particle(s), prior to the readout particle(s) being loaded, or after the readout particle(s) is loaded into the chamber. In one embodiment where a natural killer cell is employed, the natural killer cell targets one or more readout cells to which an antibody produced by an effector cell has bound. The extracellular effect may thus include lysis of the one or more readout cells by the natural killer cell. Lysis can be measured by viability dyes, membrane integrity dyes, release of fluorescent dyes, enzymatic assays, etc.

In some embodiments, the extracellular effect is neutralization of an accessory particle (or accessory reagent) operable to affect the readout particle, e.g. a cytokine (accessory particle) operable to stimulate a response of the at least one readout cell. For example, cytokine-secreting cells operable to affect the readout particle cells may further be provided to the chamber. Neutralization of the secreted cytokines by an effector cell may be detected as the absence of the expected effect of the cytokine on the readout cell, e.g. proliferation. In another embodiment, the accessory particle is a virus operable to infect the readout cell(s), and neutralization of the virus is detected as the reduced infection of readout cells by the virus.

In some embodiments, the extracellular effect of one effector cell induces activation of a second effector cell (e.g., secretion of antibodies or cytokine by the second effector cell), which can then elicit a response in the at least one readout cell.

In one embodiment, the cell population isolated in a chamber of one of the devices provided herein comprises an ASC that secretes a monoclonal antibody. In one embodiment, a readout bead based assay is used in a method of detecting the presence of an ASC secreting the antibody amid a background of one or more additional cells not secreting the antibody. For example, a bead based assay is employed in one embodiment, in a method of detecting an ASC within a cell population, whose antibody binds a target epitope of interest, in the presence of one or more additional ASCs that secrete antibodies that do not bind the target epitope of interest.

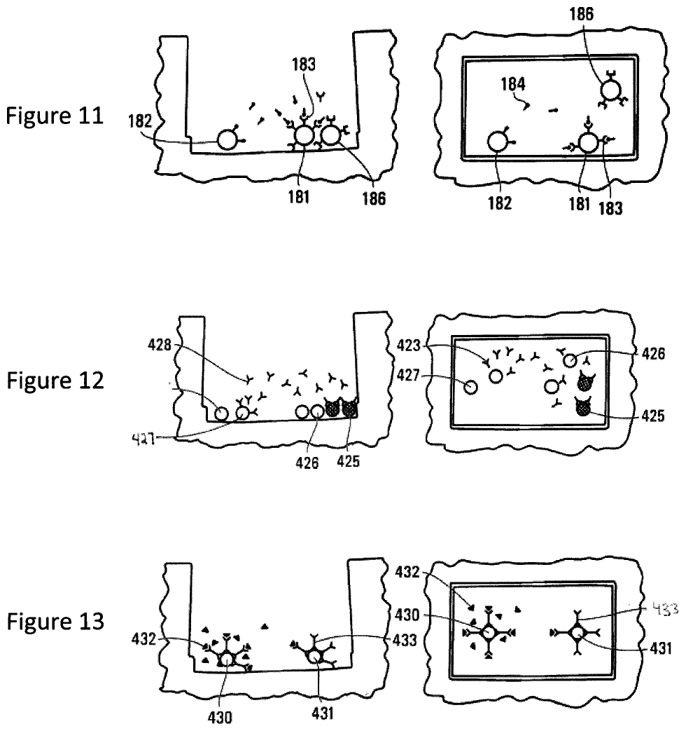

In another embodiment, the ability of an antibody to bind specifically to a target cell is assessed. Referring to FIG. 11, the assay includes at least two readout particles, e.g., readout cells 181 and 186, in addition to at least one effector cell 182 (ASC). Readout cell 181 expresses a known target epitope of interest, i.e., target epitope 183, on its surface (either naturally or through genetic engineering) while readout cell 186 does not. The two types of readout cells 181 and 186 may be distinguishable from themselves and the effector cell 182 by a distinguishable fluorescent marker, other stain or morphology. Effector cell 182 secretes antibody 184 in the same chamber as readout cells 181 and 186. Antibody 184 secreted by effector cell 182 binds to readout cell 181 via target epitope 183, but does not bind to readout cell 186. A secondary antibody is used to detect the selective binding of antibody 184 to readout cell 181. The assay chamber is then imaged to determine if antibody 184 binding to the readout cell 181 and/or readout cell 186 has occurred.

Such an assay may also be used to assess the location of antibody binding on or inside the readout cell(s) using high resolution microscopy. In this embodiment, the readout particles include different particle types (e.g., cell types) or particles/cells prepared in different ways (for example, by permeabilization and fixation) to assess binding specificity and/or localization. For example, the assay can be used to identify antibodies that bind the natural conformation of a target on live cells and the denatured form on fixed cells. The assay may alternatively be used to determine the location of an epitope on a target molecule by first blocking other parts of the molecule with antibodies against known epitopes, with different populations of readout particles having different blocked epitopes.

In another embodiment, individual heterogeneous readout particle populations, (e.g., a readout cell population comprising malignant and normal cells) and individual cell populations, wherein at least one of the individual cell populations comprises an effector cell (e.g., an ASC), are provided to a plurality of assay chambers (e.g., greater than 1000 chambers) of one of the devices provided herein. For example, referring to FIG. 12, binding to one or more malignant readout cells 425 and absence of binding to healthy readout cells 426 in the population of readout cells is used to identify a cell population containing one or more effector cells producing an antibody of interest, i.e., effector cell 427 producing antibody 428 specific to one or more of the malignant cells in the population. The two types of readout cells 425 and 426 within a chamber are distinguishable by at least one property, e.g., fluorescence, varying levels of fluorescence intensity, morphology, size, surface staining, location in the assay chamber. The cells are then incubated within the individual chambers and imaged to determine if one or more of the chambers includes a cell population that displays the extracellular effect, i.e., an antibody that binds to a malignant readout cell but not a healthy readout cell.

If present, the cell population containing the one or more ASCs that secretes an antibody that binds a malignant readout cell 425, but not the healthy readout cells 426, can then be recovered to retrieve the sequences of the antibodies within the chamber, or to perform other downstream assays on the individual cells within the population, for example, an assay to determine which ASC in the population has the desired binding property. Accordingly, novel functional antibodies discovered by one or more of the methods described herein, are provided. The epitope on the malignant readout cell 425 may be known or unknown.

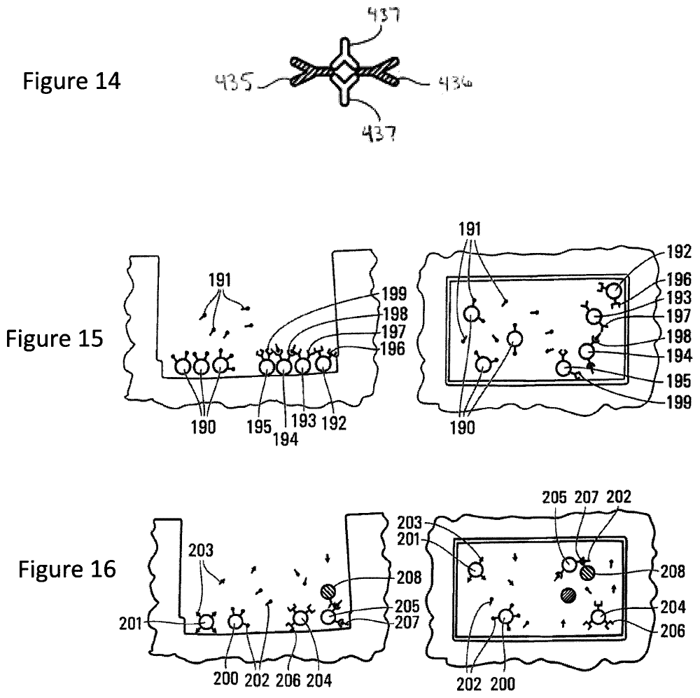

In one embodiment, a single cell type can serve as both an effector cell and a readout cell. Referring to FIG. 13, this assay is performed with effector cell 430 and readout cell 431, both functionalized to capture a molecule of interest 432 on their surfaces, for instance using tetrameric antibodies 433 directed against a surface marker and the molecule of interest 432, or an affinity-matrix on the cell to bind biotinylated antibodies. Referring to FIG. 14, a tetrameric antibody complex consists of an antibody (A) 435 that binds the cells and an antibody (B) 436 that binds antibodies secreted from the cells, wherein antibodies A and B are connected by two antibodies 437 that bind the Fc portion of antibodies A and B. Such tetrameric antibody complexes have been described in the art (Lansdorp et al. (1986). European Journal of Immunology 16, pp. 679-683, incorporated by reference herein in its entirety for all purposes) and are commercially available (Stemcell Technologies, Vancouver Canada). Using these tetramers, the secreted antibodies are captured and linked onto the surface of the cells, thus making the effector cells also function as readout particles. Once bound on the surface of cells these antibodies can be assayed for binding, for instance by the addition of fluorescently labeled antigen. For example, in the case where one is attempting to identify chambers that contain cells that secrete a monoclonal antibody that binds to a specific target, the antibodies that are secreted from effector cells can be captured on the surface of these effector cells, and others in the chamber, using appropriate capture agents. Referring again to FIG. 13, it is thus understood that effector cell 430 can also function as a readout cell, i.e., that the effector cell secreting a molecule of interest 432 may more efficiently capture the molecule of interest than readout cell 431.

In one embodiment, extracellular effect assays are performed in parallel in a plurality of assay chambers, wherein the readout particle populations in the chambers are heterogeneous (e.g., a heterogeneous readout cell population) and substantially homogeneous populations of cells in each chamber, where the individual effector cells within each substantially homogeneous population each produces the same antibody. In a further embodiment, the readout particles are readout cells genetically engineered to express a library of proteins or protein fragments in order to determine the target epitope of the antibody secreted by the effector cells. Referring to FIG. 15 one embodiment of the assay includes a plurality of effector cells 190 secreting antibody 191. The assay further includes a heterogeneous readout cell population comprising readout cells 192, 193, 194, and 195 displaying epitopes 196, 197, 198, and 199, respectively. Effector cells 190 secrete antibodies 191 which diffuse toward readout cells 192, 193, 194, and 195. Antibodies 191 bind to readout cell 194 via target epitope 198, but do not bind to readout cells 192, 193, or 195. A secondary antibody may be used to detect the selective binding of antibodies 191 to readout cell 194.

Cell populations that secrete antibodies 191 that bind readout cell 194 (or another epitope) can then be recovered from the device and subjected to a further assay.

In one embodiment, an assay is carried out in parallel in individual device chambers to determine whether an ASC within a cell population in a chamber activates cell lysis of a target cell, i.e., activates antibody-dependent cell-mediated cytotoxicity (ADCC). ADCC is a mechanism of cell-mediated immune defense whereby an effector cell of the immune system lyses a target cell, whose membrane-surface antigens have been bound by specific antibodies, i.e., antibodies secreted by an ASC within a particular assay chamber provided herein. Classical ADCC is mediated by natural killer (NK) cells. However, macrophages, neutrophils and eosinophils can also mediate ADCC, and can be provided herein as accessory cells to be used in an ADCC extracellular effect assay.

One embodiment of an ADCC assay provided herein includes a cell population comprising an effector cell or plurality thereof, a readout cell population (having an epitope of interest on their surfaces) and NK cells as accessory cells. The assay is run to determine if an ASC from the cell population induces the NK cells to attack the target cells and lyse them. Referring to FIG. 16, the illustrated embodiment includes cell population comprising ASCs 200 and 201 that secrete antibodies 202 and 203, respectively. The illustrated embodiment further includes a heterogeneous readout cell population comprising readout cells 204 and 205 displaying epitopes 206 and 207, respectively. ASCs 200 and 201 secrete antibodies 202 and 203 which diffuse toward readout cells 204 and 205. Antibodies 202 bind to readout cell 205 via target epitope 207, but do not bind to readout cell 204. Antibodies 203 do not bind to either of readout cells 204 and 205. NK cell 208 detects that readout cell 205 has been bound by antibodies 202 and proceeds to kill readout cell 205, while leaving unbound readout cell 204 alone.

A person skilled in the art will understand that the NK cells may be added to the chamber during or after the incubation of the effector cells with the readout cells, provided that they are added to the chamber in a manner that facilitates access (e.g., optical access) to the readout cells. NK cells (or another type of accessory cell) in one embodiment are added directly to the bottom component of a device, i.e., directly to open chambers. Alternatively, or additionally, NK cells (or another type of accessory cell) are added to chambers via microchannels formed in a top component of a device. The NK cells may be from a heterogeneous population of accessory cells, e.g., peripheral blood mononuclear cells. The NK cells may be from an animal- or human-derived cell line, and engineered to increase ADCC activity. A person skilled in the art will further understand that this assay can be performed with other hematopoietic cell types capable of mediating ADCC such as macrophages, eosinophils or neutrophils. In this case, macrophages, eosinophils or neutrophils are the accessory cells in the assay. Cell types capable of mediating ADCC can also be animal- or human-derived cell lines engineered to increase ADCC activity or to report signal upon binding antibodies on target cells. In the latter, the target cells are accessory particles while the cells mediating ADCC are the readout particles.

An ADCC extracellular effect assay can be performed on a single cell (e.g., single effector cell), a homogeneous cell population, or a heterogeneous cell population as depicted in FIG. 16. Similarly, an ADCC assay can be performed with a single readout cell, a homogeneous readout cell population, or a heterogeneous readout cell population, as depicted in FIG. 16. However, in many instances it is desirable to perform an ADCC assay with a plurality of readout cells to avoid the detection of false positives resulting from the random death of a readout cell.

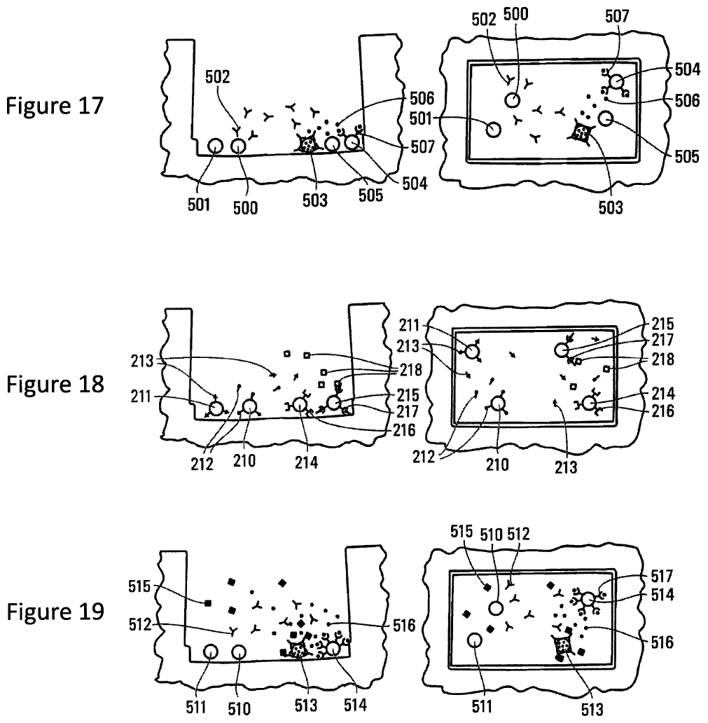

Cell lysis, in one embodiment, is quantified by a clonogenic assay, by the addition of a membrane integrity dye, by the loss of intracellular fluorescent molecules or by the release of intracellular molecules in solution. The released biomolecules are measurable directly in solution or captured onto readout particles for measurement. In some cases, additional accessory molecules are added, such as a substrate for a redox assay or a substrate for an enzymatic assay. Referring to FIG. 17, for example, a cell population comprising effector cell 500 secreting first biomolecule 502 and a second effector cell 501 that does not secrete first biomolecule 502, is incubated in the presence of a heterogeneous readout particle population, including readout cell 503 and readout particle 504, and an accessory particle (e.g., natural killer cell 505). Binding of first biomolecule 502 to readout cell 503 elicits the recruitment of natural killer cell 505 that causes readout cell 503 to lyse. Upon cell lysis, second biomolecule 506 is released from readout cell 503 and captured on readout particle 504, a different type of readout particle that is functionalized to capture second biomolecules 506, e.g., via molecule 507. Molecule 507, in one embodiment, is a protein such as an antibody or an enzyme, a reactive group and/or a nucleic acid. Captured second biomolecule 506 can be any molecule present in readout cell 503 such as a protein, enzyme, carbohydrate or a nucleic acid. Binding of the second biomolecule 506 to readout particle 504, in one embodiment, is quantified using a fluorescence assay, a colorimetric assay, a bioluminescence assay or a chemoluminescence assay. The assay is performed directly on readout particle 504 or indirectly in the surrounding solution, e.g., if captured biomolecule 506 is an enzyme that converts a substrate into a product with different optical properties. The assay is carried out in multiple chambers of one of the devices provided herein to determine if any of the chambers comprises an effector cell that secretes a biomolecule (e.g., antibody) that induces cell lysis.

ADCC assays are known in the art and components are commercially available. For example, the Guava Cell Toxicity Kit for Flow Cytometry (Millipore), the ADCC Reporter Bioassay Core Kit (Promega), the ADCC Assay (GenScript), the LIVE/DEAD Cell Mediated Cytotoxicity Kit (Life Technologies) and the DELFIA cell toxicity assays can be utilized.

In another embodiment, the extracellular effect assay is a complement-dependent cytotoxicity (CDC) assay. In one CDC embodiment, a method is provided for identifying the presence of an ASC (or secreted antibody of an ASC) within a cell population that binds to a readout cell in the presence of soluble factors necessary and/or sufficient to induce lysis of the readout cell via the classic complement pathway. Accordingly, the assay is to determine in one embodiment, whether an antibody secreted by an ASC stimulates lysis of one or more target cells by the classic complement pathway.

A CDC assay includes at least one effector cell and at least one readout cell, and one CDC embodiment is depicted in FIG. 18. The embodiment includes a cell population that includes effector cell 210 and effector cell 211 secreting antibodies 212 and 213, respectively. The illustrated embodiment further includes a heterogeneous readout cell population comprising readout cell 214 and readout cell 215 displaying epitopes 216 and 217, respectively. Effector cells 210 and 211 secrete antibodies 212 and 213 which diffuse toward readout cells 214 and 215. Antibodies 212 bind to readout cell 215 via target epitope 217, but do not bind to readout cell 214. Antibodies 213 do not bind to either of readout cells 214 and 215. Enzyme C1 218, an accessory particle, and one of the soluble factors necessary to induce lysis of cells via the classic complement pathway, binds to the complex of readout cell 215 with antibody 212 while leaving unbound readout cell 214 alone. Binding of enzyme C1 208 to the complex of readout cell 215 with antibody 212 triggers the classic complement pathway involving additional soluble factors necessary to induce lysis of cells via the class complement pathway (not shown), leading to the rupture and death of readout cell 215.

The soluble factors necessary and/or sufficient to induce lysis of the readout cells (i.e., the accessory particles necessary for the assay) are added during or after the incubation of the effector cells with the readout cells, provided that they are added to the chamber in a manner that facilitates access to the readout cells. Such accessory particles can be added to chambers via a top device component or a bottom device component, as described above. CDC assays provided herein can be performed on a single effector cell, a homogeneous effector cell population, or a heterogeneous cell population as depicted in FIG. 18. Similarly, the CDC assay can be performed with a single readout cell, a homogeneous readout cell population or a heterogeneous readout cell population, as depicted in FIG. 18. However, it is desirable in many instances to perform the CDC assay with a readout cell population to avoid the detection of false positives resulting from the random death of a readout cell.

Cell lysis by the complement pathway is quantified according to methods known to those of skill in the art. For example, cell lysis is quantified by a clonogenic assay, by the addition of a membrane integrity dye, by the loss of intracellular fluorescent molecules or by the release of intracellular molecules in solution. The released biomolecules are measured directly in solution or captured onto readout particles. In some cases, additional accessory molecules may be added such as a substrate for a redox assay or a substrate for an enzymatic assay. Referring to FIG. 19, for example, a cell population, including an effector cell 510 secreting a first biomolecule 512 and a second effector cell 511 that does not secrete first biomolecule 512, is incubated in the presence of heterogeneous readout particles, e.g., readout cell 513 and readout particle 514, in the presence of accessory particle 515 (e.g., complement proteins). Binding of biomolecule 512 to readout cell 513 in the presence of accessory particles 515 causes readout cell 513 to lyse. Upon cell lysis, second biomolecule 516 is released and captured on a readout particle 514, a second type of readout particle that is functionalized to capture biomolecule 516, e.g., via molecules 517. Molecules 517 may be one or more types of molecule such a protein, an antibody, an enzyme, a reactive group and/or a nucleic acid. Captured biomolecule 516 is not limited to type. Rather, captured biomolecule 516 is any molecule present in readout cell 513 such as protein, enzyme, dye, carbohydrate or nucleic acid. Binding of the second biomolecule 516 to readout particle 514 is quantified using a fluorescence assay, a colorimetric assay, a bioluminescence assay or a chemoluminescence assay. It is understood that the assay may be performed directly on readout particle 514 or indirectly in the surrounding solution, for instance if captured biomolecule 516 is an enzyme that converts a substrate into a product with different optical properties.

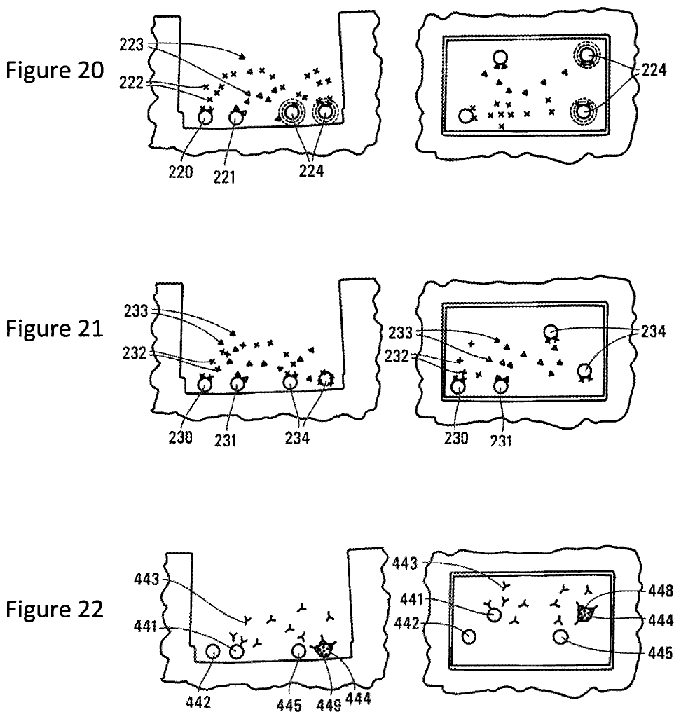

In another embodiment, an assay is provided to determine whether an effector cell, alone or within a cell population modulates cell growth. Specifically, the assay is used to determine whether the effector cell secretes a biomolecule, e.g., a cytokine or antibody that modulates the growth rate of readout cells. Referring to FIG. 20, the illustrated embodiment includes a cell population comprising effector cell 220 and effector cell 221 secreting biomolecules 222 and 223, respectively. The illustrated embodiment further includes a homogeneous readout cell population comprising readout cell 224. Effector cells 220 and 221 secrete biomolecules 222 and 223 which diffuse toward readout cells 224. Biomolecule 222 binds to readout cell 224 to induce growth of readout cell 224 (represented by perforated lines), whereas biomolecule 223 do not bind to readout cell 224. Microscopic imaging of the chamber is used to assess the growth of the readout cells 224 relative to cells in other chambers which are not exposed to the biomolecules.

A cell growth modulation assay can be performed using a cell population that optionally comprises one or more effector cells. As noted above, in some embodiments, not all cell populations will contain effector cells because of their rarity and/or difficulty to be enriched for in a starting population that is initially loaded onto one of the devices provided herein. The present invention allows for the identification of these rare cells by identifying cell populations that comprise one or more effector cells.

The cell growth modulation assay can also be performed with a single readout cell, or a heterogeneous readout cell population in a single chamber. However, in many instances, it is desirable to perform the cell growth modulation assay with a homogeneous readout cell population to permit a more accurate measurement of growth rate.

The cell growth modulation assay, in one embodiment, is adapted to screen for cells producing biomolecules that inhibit cell growth. In another embodiment, the method is adapted to screen for cells producing molecules that modulate, i.e., increase or decrease, proliferation rates of readout cells. Growth rate, in one embodiment, is measured by manual or automated cell count from light microscopy images, total fluorescence intensity of cell expressing a fluorescence, average fluorescence intensity of cells labeled with a dilutive dye (e.g. CFSE), nuclei staining or some other method known to those of skill in the art.

Commercially available assay to measure proliferation include the alamarBlue® Cell Viability Assay, the CellTrace™ CFSE Cell Proliferation Kit and the CellTrace™ Violet Cell Proliferation Kit (all from Life Technologies), each of which can be used with the methods and devices described herein.

In another embodiment, an apoptosis assay is provided to select a cell population comprising one or more effector cells that induces apoptosis of another cell, i.e., a apoptosis of a readout cell or an accessory cell. In a further embodiment, the method is used to identify the presence of an effector cell that secretes a biomolecule, e.g., a cytokine or an antibody that induces apoptosis of a readout cell or accessory cell. Referring to FIG. 21, the assay is carried out in a chamber that includes a cell population comprising effector cell 230 secreting biomolecule 232 and effector cell 231 secreting biomolecule 233. The chamber further includes a homogeneous readout cell population comprising readout cell 234. Effector cell 230 and effector cell 231 secrete biomolecules 232 and 233, which diffuse toward readout cells 234. Biomolecule 232 binds to readout cell 234 and induces apoptosis of readout cell 234, whereas biomolecule 233 does not bind to the readout cell. Microscopic imaging of the chamber, in one embodiment, is used to assess apoptosis using, potentially with the inclusion of stains and other markers of apoptosis that are known in the art (e.g., Annexin 5, terminal deoxynucleotidyl transferase (TdT)-mediated dUTP nick end labeling, mitochondrial membrane potential disruption, etc.). In one embodiment, cell death using commercially available dyes or kits is measured, for example with propidium iodide (PI), LIVE/DEAD® Viability/Cytotoxicity Kit (Life Technologies) or LIVE/DEAD® Cell-Mediated Cytotoxicity Kit (Life Technologies).

An apoptosis assay, in one embodiment, is performed on a cell population comprising a single effector cell, a cell population optionally comprising one or more effector cells or a cell population comprising one or more effector cells. In one embodiment, the apoptosis assay is performed with a single readout cell, or a heterogeneous readout cell population. However, in many instances, it is desirable to perform the apoptosis assay with a homogeneous readout cell population to permit a more accurate assessment of apoptosis.

In another embodiment, the devices and assays provided herein are used to identify an effector cell that secretes a biomolecule, e.g., a cytokine or antibody that induces autophagy of a readout cell. One embodiment of this method is shown in FIG. 22. In this embodiment, a cell population comprising effector cell 441 and effector cell 442, wherein effector cell 441 secretes biomolecule 443, is subjected to the assay. The illustrated embodiment further includes a heterogeneous readout cell population including first readout cell 444 displaying a target epitope 449 and a second readout cell 445 lacking the target epitope. Effector cell 441 secretes biomolecules 443, which diffuses toward first readout cell 444 and second of readout cell 445. Biomolecule 443 binds to first readout cell 444 and induces autophagy of first readout cell 444, whereas biomolecules 443 does not bind to the second readout cell 445. Microscopic imaging of the chamber, in one embodiment, is used to assess autophagy using cell lines engineered with autophagy reporters that are known in the art (e.g., FlowCellect™ GFP-LC3 Reporter Autophagy Assay Kit (U20S) (EMD Millipore), Premo™ Autophagy Tandem Sensor RFP-GFP-LC3B Kit (Life Technologies)).

In one embodiment, an autophagy assay is performed on a cell population comprising a single effector cell, a cell population optionally comprising one or more effector cells or a cell population comprising one or more effector cells. In one embodiment, an autophagy assay is performed with a single readout cell, or a heterogeneous readout cell population, or a homogeneous readout cell population. The assay, in one embodiment, is performed with a homogeneous readout cell population.

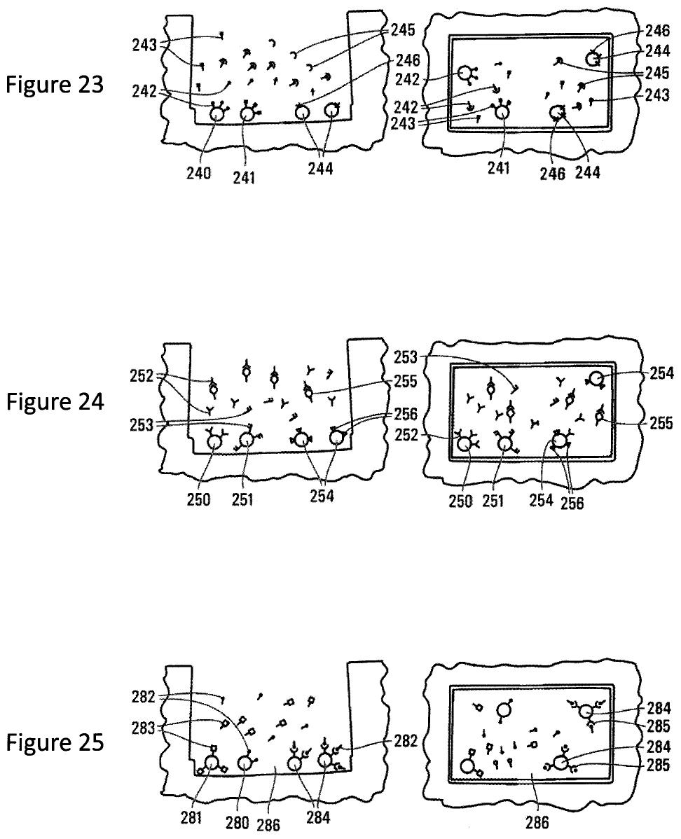

In another embodiment, a method is provided for identifying the presence of an effector cell or to select an effector cell that secretes a biomolecule, e.g., an antibody, that interferes with the ability of a known biomolecule, e.g., a cytokine, to induce a response in a readout cell. The response is not limited by type. For example, the response in one embodiment is selected from cell death, cell proliferation, expression of a reporter, change in morphology, or some other response selected by the user of the method. One embodiment of the method is provided in FIG. 23. Referring to FIG. 23, the illustrated embodiment includes a cell population comprising effector cell 240 and effector cell 241 secreting biomolecules 242 and 243, respectively. The illustrated embodiment further includes a homogeneous readout cell population comprising readout cell 244. Effector cells 240 and 241 secrete antibodies 242 and 243, which diffuse into the medium within the chamber. The chamber is pulsed with cytokines 245, which normally have a known effect on readout cells 244. Antibodies 242 bind to cytokines 245, and thereby prevent them from binding to readout cells 244. Accordingly, the expected response is not observed, indicating that one of effector cell 240 and 241 is secreting an antibody capable of neutralizing the ability of cytokine 245 to stimulate the readout cells 244 to undergo a response.

In one embodiment, a cytokine neutralization assay is used to identify the presence of a cell population comprising an effector cell that produces a biomolecule targeting a receptor for the cytokine, present on the readout cell. In this case, binding of an antibody, e.g., antibody 242 to receptors 246 for cytokine 245 on readout cells 244 blocks the interaction of the cytokine and the receptor, so that no response would be stimulated. The cytokine receptor, in another embodiment, is “solublized” or “stabilized,” for example, is a cytokine receptor that has been engineered via the Heptares StaR® platform.

The response to the cytokine, in one embodiment, is ascertained by microscopic measurements of the associated signaling as known in the art including, but not limited to cell death, cell growth, the expression of a fluorescent reporter protein, the localization of a cellular component, a change in cellular morphology, motility, chemotaxis, cell aggregation, etc. In one embodiment, the response of chambers with effector cells are compared to chambers lacking effector cells to determine whether the response is inhibited. If a response is inhibited, the effector cells within the chamber are harvested for further analysis.

In one embodiment, a cytokine assay is performed within an individual device chamber on a cell population comprising a single effector cell, a cell population optionally comprising one or more effector cells or a cell population comprising one or more effector cells. The method is carried out in parallel in a plurality of chambers of a single device on a plurality of cell populations. In one embodiment, the cytokine assay is performed with a single readout cell, or a heterogeneous readout cell population. In one embodiment, the method is carried out with a homogeneous readout cell population to permit a more accurate assessment of stimulation, or rather lack thereof, of the readout cells.

Examples of commercially available cytokine-dependent or cytokine-sensitive cell lines for such assays include, but are not limited to TF-1, NR6R-3T3, CTLL-2, L929 cells, A549, HUVEC (Human Umbilical Vein Endothelial Cells), BaF3, BW5147.G.1.4.OUAR.1, (all available from ATCC), PathHunter® CHO cells (DiscoveRx) and TANGO cells (Life Technologies). A person of skill in the art will understand that primary cells (e.g., lymphocytes, monocytes) may also be used as readout cells for a cytokine assay.

In one embodiment, a signaling assay is used to identify a cell population comprising one or more effector cells that secretes a molecule (e.g., an antibody or a cytokine) that has agonist activity on a receptor of a readout cell. Upon binding to the receptor, the effect on the readout cell population may include activation of a signaling pathway visualized by expression of a fluorescent reporter, translocation of a fluorescent reporter within a cell, a change in growth rate, cell death, a change in morphology, differentiation, a change in the proteins expressed on the surface of the readout cell, etc. Several engineered reporter cell lines are commercially available and can be used to implement such an assay. Examples include PathHunter Cells® (DiscoverRx), TANGO™ cells (Life Technologies) and EGFP reporter cells (ThermoScientific). In one embodiment the receptor is a GPCR receptor and the readout cell is a cell that has been engineered to express a transcriptional reporter in response to cyclic AMP secondary message. In one embodiment that receptor is an ion channel and the readout cells are assayed for an effect by using a calcium-sensitive fluorescent dye.

In one embodiment, a virus neutralization assay is carried out to identify and/or select a cell population comprising one or more effector cells that secretes a biomolecule, e.g., an antibody that interferes with the ability of a virus to infect a target readout cell or target accessory cell. One embodiment of this method is shown in FIG. 24. Referring to FIG. 24, the illustrated embodiment includes a cell population comprising effector cell 250 and effector cell 251 secreting biomolecules, e.g., antibodies 252 and 253, respectively. The illustrated embodiment further includes a homogeneous readout cell population comprising readout cell 254. Effector cells 250 and 251 secrete biomolecules, e.g., antibodies 252 and 253, which diffuse into the medium within the chamber. The chamber is then pulsed with virus 255 (accessory particle), which normally infects readout cells 254. Antibody 252 or 253 binds to virus 255, and thereby prevents the virus from binding to readout cell 254. If the expected infection is not observed, it is concluded that one of effector cells 250 or 251 secretes an antibody capable of neutralizing virus 255.

A virus neutralization assay can be used to identify an effector cell that produces a biomolecule which binds a receptor for the virus on the readout cell. In this case, binding of an antibody, e.g., antibody 252 to receptor 256 of virus 255 on readout cell 254 blocks the interaction of the virus and the receptor, so that no infection would be observed.

Assessment of viral infection may be done using methods known in the art. For example, the virus can be engineered to include fluorescent proteins that are expressed by the readout cell following infection, the expression of fluorescent proteins within the readout cell that are upregulated during viral infection, the secretion of proteins from a readout cell or accessory cell, which are captured and measured on readout particles that are increased during viral infection, the death of the of a readout cell or accessory cell, the change in morphology of a readout cell or accessory cell, and/or the agglutination of readout cells.

In one embodiment, the extracellular effect assay is a virus neutralization assay. In one embodiment, the virus neutralization assay is performed with a single readout cell, or a heterogeneous readout cell population. In one embodiment, the method is carried out with a homogeneous readout cell population to permit a more accurate assessment of the stimulation, or rather lack thereof, of the readout cells to undergo the response. Commercially available cell lines for virus neutralization assays are MDCK cells (ATCC) and CEM-NKR-CCR5 cells (NIH Aids Reagent Program) can be used with the methods and devices described herein.

In another embodiment, the extracellular effect assay is an enzyme neutralization assay, and is performed to determine whether an effector cell displays or secretes a biomolecule that inhibits a target enzyme. One embodiment of the method is provided in FIG. 25. Referring to FIG. 25, the illustrated embodiment includes a cell population comprising effector cell 280 and effector cell 281 secreting biomolecules, e.g., proteins 282 and 283, respectively. The illustrated embodiment further includes a homogeneous readout particle population, e.g., beads 284, to which a target enzyme 285 is conjugated. However, in another embodiment, the target enzyme 285 is linked to the surface of the device, or is soluble. Proteins 282 and 283 diffuse through the medium and protein 282 binds to target enzyme 285, thereby inhibiting its activity, whereas protein 283 does not bind to the target enzyme. Detection of the enzymatic activity, or rather lack thereof, on a substrate present in the chamber, in one embodiment, is assessed by methods known in the art including but not limited to fluorescent readouts, colorometric readouts, precipitation, etc.

In another embodiment, an enzyme neutralization assay is performed on a cell population comprising a single effector cell, a cell population optionally comprising one or more effector cells or a cell population comprising one or more effector cells, per individual chamber. In one embodiment, the enzyme neutralization assay is performed with a single readout particle in an individual chamber. In one embodiment, an enzyme neutralization assay is carried out on a plurality of cell populations to identify a cell population that exhibits a variation in assay response.

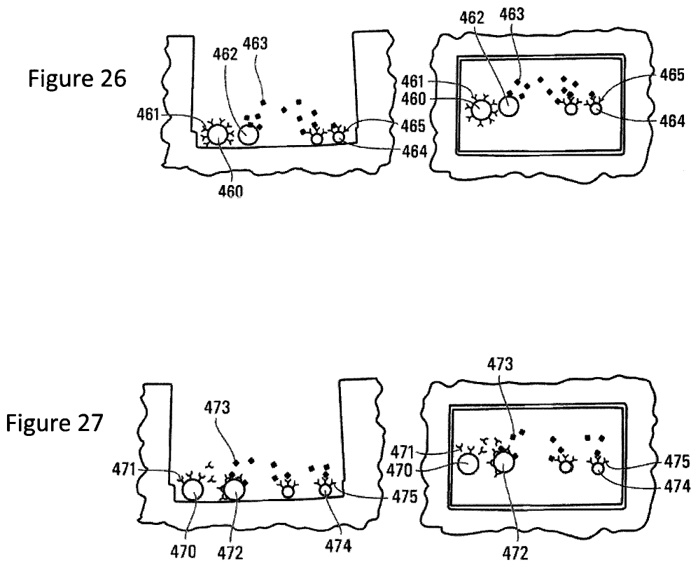

In another embodiment, an assay is provided for identifying the presence of an effector cell that displays or secretes a molecule that elicits the activation of a second type of effector particle, which in turn secretes a molecule that has an effect on a readout particle. Accordingly, in this embodiment, individual cell populations are provided to individual assay chambers. One embodiment of this method is provided in FIG. 26. Referring to FIG. 26, the illustrated embodiment includes a cell population that includes one effector cell 460 that displays a molecule 461 on its surface (e.g., an antibody, a surface receptor, a major histocompatibility complex molecule, etc.), which activates an adjacent effector cell of different type, in this case effector cell 462, which induces the secretion of another type of molecule 463 (e.g., cytokine, antibody) that is captured by the readout particle 464. In this example, the readout particles 464 are functionalized with an antibody 465 or receptor specific to the secreted molecule 463.

In another embodiment, the effector cell, upon activation by an accessory particle, may exhibit changes in phenotype such as proliferation, viability, morphology, motility or differentiation. In this case the effector cell is also a readout particle. This effect can be caused by the accessory particles and/or by autocrine secretion of proteins by the activated effector cells.

Referring to FIG. 27, the illustrated embodiment includes a cell population comprising effector cell 470 that secretes a molecule 471 (e.g., antibody, cytokine, etc.), which activates a second effector cell of different type, in this case effector cell 472. Effector cell 472, once activated, secretes molecule 473 (e.g., cytokine, antibody) that is captured by readout particles 474. In this example, the readout particles 474 are functionalized with an antibody 475 or receptor specific to the secreted molecule 473.

As provided herein, monoclonal antibodies with low off-rates are detectable in the presence of a large background (in the same chamber) of monoclonal antibodies that are also specific to the same antigen but which have faster off-rates. However, affinity is also measurable with the devices and methods provided herein, and therefore, on-rate can also be measured. These measurements depend on the sensitivity of the optical system as well as the binding capacity of the capture reagents (e.g., beads). To assay for specificity, the capture reagents (readout particles) may be designed to present the epitope of interest so that it only binds antibodies with a desired specificity.

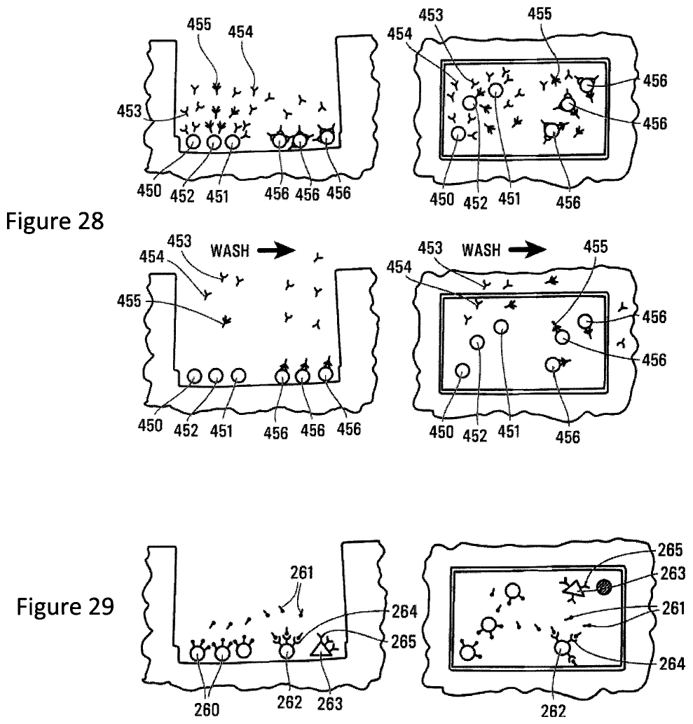

Referring to FIG. 28, the illustrated embodiment includes a homogeneous cell population secreting antibodies specific for the same antigen but with different affinities. This assay is used to identify an effector cell that produces an antibody with high affinity. Effector cells 450 and 451 secrete antibodies 453 and 454 with low affinities for a target epitope (not shown) while effector cell 452 secretes antibodies 455 with higher affinity for the target epitope. Antibodies 453, 454, and 455 are captured by a homogeneous readout particle population comprising readout bead 456. The readout beads are then incubated with a fluorescently labeled antigen (not shown), which binds to all antibodies. Upon washing with a non-labeled antigen (not shown), the fluorescently labeled antigen remains only if readout beads display high-affinity antibody 455 on their surfaces.

Referring to FIG. 29, effector cells 260 secreting biomolecule, e.g., antibody 261 are provided in a chamber. The chamber further includes a readout particle population comprising optically distinguishable readout particles, e.g., beads 262 and 263 displaying different target epitopes 264 and 265, respectively. Antibody 261 diffuses in the chamber, where it binds to epitope 264, but not 265. Preferential binding of antibody 261 to epitope 264 in one embodiment, is observed in terms of fluorescence of bead 262 but not bead 263.

In the illustrated embodiment, beads 262 and 263 are optically distinguishable by shape, to assess cross-reactivity. However, readout particles can also be distinguishable by other means, e.g., one or more characteristics such as fluorescence labeling (including different fluorescence wavelengths), varying levels of fluorescence intensity (e.g., using Starfire™ beads with different fluorescence intensities, morphology, size, surface staining and location in the assay chamber).

In one embodiment, beads 262 and 263 are optically distinguishable via the use of different color fluorophores.

In one embodiment, specificity is measured by inclusion of an antibody that competes with the antibody secreted by the effector cell for binding of the target epitope. For instance, in one embodiment the presence of a secreted antibody bound to a readout particle displaying the antigen is identified with a fluorescently labeled secondary antibody. The subsequent addition of a non-labeled competing antibody generated from a different host and known to bind a known target epitope on the antigen results in decreased fluorescence due to displacement of the secreted antibody only if the secreted antibody is bound to the same target epitope as the competing antibody. Alternatively, specificity is measured by adding a mixture of various antigens that compete with binding of the secreted antibody to the target epitope if the secreted antibody has low specificity. Alternatively, specificity is measured by capturing secreted antibody on a bead and then using differentially labeled antigens to assess the binding properties of the secreted antibody.

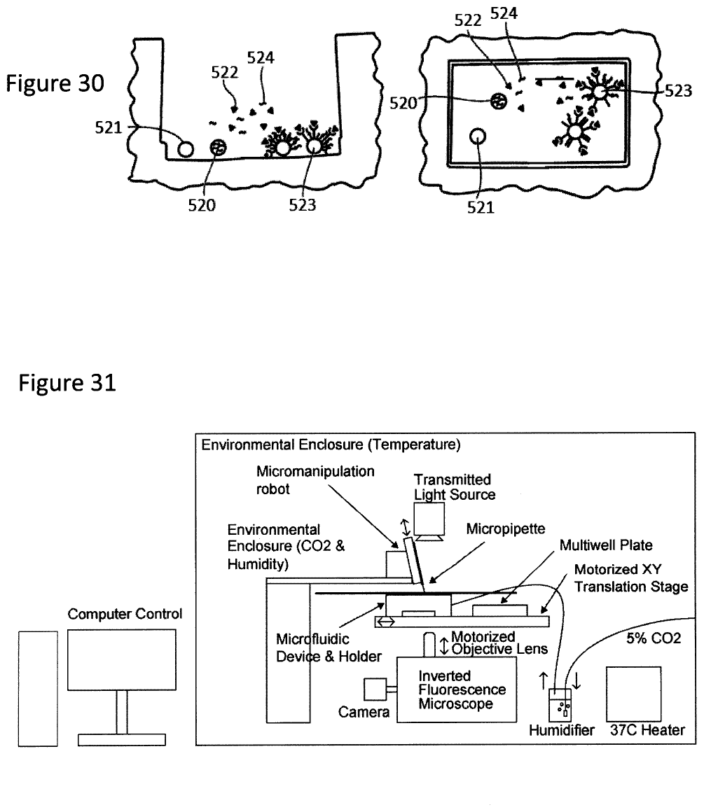

In one embodiment, a method for identifying the presence of an effector cell secreting a biomolecule is coupled with the analysis of the presence or absence of one or more intracellular compounds of the effector cell. As described throughout, the identification of the effector cell in one embodiment initially comprises the identification of a cell population comprising the effector cell. Referring to FIG. 30, a cell population comprising at least one effector cell type 520 that secretes a biomolecule of interest 522 (e.g., an antibody, or a cytokine) and another effector cell type 521 that does not secrete the biomolecule of interest, is incubated in the presence of a readout particle population comprising readout particle 523 functionalized to capture the biomolecule of interest. After an incubation period, the cell population including effector cell types 520 and 521 is lysed to release the intracellular contents of the cells in the population. Readout particles 523 are also functionalized to capture an intracellular biomolecule of interest 524 (e.g., a nucleic acid, a protein such as an antibody). Cell lysis can be achieved by different methods known to those of skill in the art.

In one embodiment, methods are provided for identifying polyclonal mixtures of secreted biomolecules with desirable binding properties. Assays may be performed with heterogeneous mixtures of effector cells producing antibodies with known affinities for a target epitope, target molecule, or target cell type. Binding of the target in the context of the mixture can then be compared to binding of the target in the context of the individual effector cells alone to determine, for example, if mixtures provide enhanced effects.

In one embodiment of the assays provided herein, after readout particles are incubated with a cell population in an assay chamber, a fluorescent measurement is taken to determine if a cell within the population demonstrates the extracellular effect. In this embodiment, the readout particle population (or subpopulation thereof) is fluorescently labeled and a change in fluorescence is correlated with the presence and/or size of the extracellular effect. The readout particle population can be directly or indirectly labeled. As will be appreciated by one skilled in the art, assays are carried out that provide readout particles and effector cells in one focal plane, to allow for accurate imaging and fluorescent measurement.

In one embodiment, readout particle responses are monitored using automated high resolution microscopy. For example, imaging can be monitored by using a 20× (0.4 N.A.) objective on an Axiovert 200 (Zeiss) or DMIRE2 (Leica) motorized inverted microscope. Using the automated microscopy system provided herein allows for complete imaging of an array comprising about 4000 chambers, including 1 bright-field and 3 fluorescent channels, in approximately 30 minutes. This platform can be adapted to various device designs, as described in Lecault et al. (2011). Nature Methods 8, pp. 581-586, incorporated by reference herein in its entirety for all purposes. Importantly, the imaging methods used herein achieve a sufficient signal in effect positive chambers while minimizing photodamage to cells.

In one embodiment, the extracellular effect assays provided herein benefit from long-term cell culture, and therefore require that the effector cells maintained in the device are viable and healthy cells. It will be appreciated that in embodiments where readout cells or accessory cells are used in an extracellular effect assay, that they are also maintained in a healthy state and are viable and healthy. The fluidic architectures provided herein enable control of medium conditions to maintain effector and readout cell viability so that extracellular effect assays can be carried out. For example, some cell types require autocrine or paracrine factors that depend on the accumulation of secreted products. For instance, CHO cell growth rates are highly dependent on seeding density. Confining a single CHO cell in a 4-nL chamber corresponds to a seeding density of 250,000 cells/ml, which is comparable to conventional macroscale cultures. Because these cells thrive at high seeding densities, CHO cells may not require perfusion for multiple days. However, other cell types, in particular those that are cytokine-dependent (e.g., ND13 cells, BaF3 cells, hematopoietic stem cells), typically do not reach high concentrations in macroscale culture and may require frequent feeding in the microfluidic device to prevent cytokine depletion. Cytokines may be added to the medium or produced by feeder cells. For instance, bone marrow derived stromal cells and eosinophils have been shown to support the survival of plasma cells because of their production of IL-6 and other factors (Wols et al. (2002). Journal of Immunology 169, pp. 4213-21; Chu et al. (2011), Nature Immunology 2, pp. 151-159, incorporated by reference herein in their entireties). In this case, the perfusion frequency can be modulated to allow sufficient accumulation of paracrine factors while preventing nutrient depletion.

As provided throughout, one aspect of the present invention provides a method for determining whether a cell population optionally comprising one or more effector cells (e.g., ASCs) exerts an extracellular effect on a readout particle (e.g., a cell comprising a cell surface receptor). In one embodiment, the effector cell is an ASC. The extracellular effect, in one embodiment, is the inhibition (antagonism) or activation (agonism) of a cell surface receptor (e.g., the agonist and/or antagonist properties of an antibody secreted by an antibody secreting cell) on a readout cell. In a further embodiment, the extracellular effect is an agonist or antagonist effect on a transmembrane protein, which in a further embodiment is a G protein coupled receptor (GPCR), a receptor tyrosine kinase (RTK), an ion channel or an ABC transporter. In a further embodiment, the receptor is a cytokine receptor. An extracellular effect on other metabotropic receptors besides GPCRs and RTKs can be assessed. For example, an extracellular effect on a guanylyl cyclase receptor can be assessed by incubating a cell population with a readout cell population expressing the guanylyl cyclase receptor.

In embodiments where a readout cell is used, the readout cell can be alive or fixed. The extracellular effect in one embodiment, is an effect on an intracellular protein of the fixed readout cell. Extracellular effects can also be measured on extracellular proteins of an alive or fixed readout cell, or a secreted protein of a viable readout cell.

In another embodiment, a readout cell expresses a serine/threonine kinase receptor or a histidine-kinase associated receptor and the extracellular effect assay measures binding, agonism or antagonism of the cell receptor.

In embodiments where a particular receptor (e.g., receptor serine/threonine kinase, histidine-kinase associated receptor or GPCR) is an orphan receptor, that is, the ligand for activating the particular receptor is unknown, the methods provided herein allow for the discovery of a ligand for the particular orphan receptor by performing an extracellular assay on readout cells expressing the orphan receptor, and identifying a cell population or subpopulations comprising an effector cell responsible for eliciting a variation of an extracellular effect (e.g., agonism or antagonism of the orphan receptor as compared to a control value) on the readout cell expressing the orphan receptor.

In one embodiment, the cell surface protein on the surface of a readout cell is a transmembrane ion channel. In a further embodiment, the ion channel is a ligand gated ion channel and the extracellular effect measured in the assay is modulation of the ion channel gating, for example, opening of the ion channel by agonist binding or closing/blocking of the ion channel by antagonist binding. The antagonist or agonist can be for example, a biomolecule (e.g., antibody) secreted by one or more effector cells. Extracellular effect assays described herein can be used to measure the extracellular effect of an effector cell on a cell expressing a ligand gated ion channel in the Cys-loop superfamily, an ionotropic glutamate receptor and/or an ATP gated ion channel. Specific examples of anionic cys-loop ion gated channels include the GABA_{A} receptor and the glycine receptor (GlyR). Specific examples of cationic cys-loop ion gated channels include the serotonin (5-HT) receptor, nicotinic acetylcholine (nAChR) and the zinc-activated ion channel. One or more of the aforementioned channels can be expressed by a readout cell to determine whether an effector cell has an extracellular effect on the respective cell by agonizing or antagonizing the ion channel. Ion flux measurements typically occur in short periods of time (i.e., seconds to minutes) and require precise fluidic control for their implementation. In one embodiment ion flux measurements are done after first identifying that secreted molecules bind to the readout cells. Ion flux measurements may be performed using a fluorescent dye that is an indicator of calcium flux. Examples of commercially available ion channel assays include Fluo-4-Direct Calcium Assay Kit (Life Technologies), FLIPR Membrane Potential Assay Kit (Molecular Devices). Ion-channel expressing cell lines are also commercially available (e.g. PrecisION™ cell lines, EMD Millipore).

In one embodiment, a readout cell expresses an ATP-binding cassette (ABC) transporter on its surface, and the extracellular effect assay comprises measuring the transport of a substrate across a membrane. The readout particles can be membrane vesicles derived from cells expressing the protein (e.g., GenoMembrane ABC Transporter Vesicles (Life Technologies)), which can be immobilized on beads. For instance, the ABC transporter could be a permeability glycoprotein (multidrug resistant protein) and the effect can be measured by the fluorescence intensity of calcein in readout cells. The Vybrant™ Multidrug Resistance Assay Kit (Molecular Probes) is commercially available to implement such an assay.

An extracellular effect assay can also be carried out via the use of a readout cell expressing an ionotropic glutamate receptor such as the AMPA receptor (class GluA), kainite receptor (class GluK) or NMDA receptor (class GluN). Similarly, an extracellular effect assay can also be carried out via the use of a readout cell expressing an ATP gated channel or a phosphatidylinositol 4-5-bisphosphate (PIP2)-gated channel.

The present invention provides a method of identifying a cell population comprising an effector cell that displays a variation in an extracellular effect. In one embodiment, the method comprises, retaining a plurality of individual cell populations in separate assay chambers, wherein at least one of the individual cell populations comprises one or more effector cells and the contents of the separate assay chambers further comprise a readout particle population comprising one or more readout particles, incubating the individual cell populations and the readout particle population within the assay chambers, assaying the individual cell populations for the presence of the extracellular effect, wherein the readout particle population or subpopulation thereof provides a readout of the extracellular effect. In one embodiment, the extracellular effect is an effect on a receptor tyrosine kinase (RTK), for example, binding to the RTK, antagonism of the RTK, or agonism of the RTK. RTKs are high affinity cell surface receptors for many polypeptide growth factors, cytokines and hormones. To date, there have been approximately sixty receptor kinase proteins identified in the human genome (Robinson et al. (2000). Oncogene 19, pp. 5548-5557, incorporated by reference in its entirety for all purposes). RTKs have been shown to regulate cellular processes and to have a role in development and progression of many types of cancer (Zwick et al. (2001). Endocr. Relat. Cancer 8, pp. 161-173, incorporated by reference in its entirety for all purposes).

Where the extracellular effect is an effect on an RTK, the present invention is not limited to a specific RTK class or member. Approximately twenty different RTK classes have been identified, and extracellular effects on members of any one of these classes can be screened for with the methods and devices provided herein. Table 2 provides different RTK classes and representative members of each class, each amenable for use herein when expressed on a readout particle, e.g., readout cell or vesicle. In one embodiment, a method is provided herein for screening a plurality of cell populations in a parallel manner in order to identify one or more populations comprising an effector cell having an extracellular effect on an RTK of one of the subclasses provided in Table 2. In one embodiment, the method further comprises recovering the one or more cell populations comprising the ASC displaying the extracellular effect to provide a recovered cell population and further subjecting the recovered cell population to one or more additional extracellular effect assays, at limiting dilution, to identify the ASC responsible for the extracellular effect. In this embodiment, the recovered population can be divided into subpopulations at limiting dilution. The additional extracellular effect assay can be carried out via one of the devices provided herein, or a benchtop assay. Alternatively, once a cell population is identified that has a cell exhibiting an extracellular effect on the RTK, the cell population is recovered, lysed and the nucleic acid amplified and sequenced. In a further embodiment, the nucleic acid comprises one or more antibody genes.

In one embodiment, the present invention relates to the identification of a cell population comprising an effector cell that antagonizes or agonizes an RTK (i.e., the extracellular effect), for example, via a secretion product, e.g., a monoclonal antibody. The effector cell is present a lone effector cell, a homogeneous environment or a heterogeneous environment (e.g., with multiple different effector cells).

TABLE 2
RTK classes and representative members of each class.

| RTK class | Representative members | Representative Ligands | Cellular Process(es) |
|---|---|---|---|
| RTK class I | ErbB-1 (epidermal growth factor | epidermal growth factor (EGF) | overexpression implicated in |
| (epidermal growth factor | receptor) | transforming growth factor α (TGF-α) | turmorigenesis |
| receptor (EGFR) family, | | heparin-binding EGF-like growth factor (HB- | |
| also known as the ErbB | | EGF) | |
| family) | | amphiregulin (AREG) | |
| | | betacellulin | |
| | | epigen | |
| | | epiregulin | |
| | ErbB-2 (human epidermal growth | Monoclonal antibody trastuzumab (Herceptin) | turmorigenesis (e.g., breast, |
| | factor receptor 2 (HER2)/ | | ovarian, stomach, uterine) |
| | cluster of differentiation 340 | | |
| | (CD340)/ | | |
| | proto-oncogene Neu) | | |
| | ErbB-3 (human epidermal growth | Neuregulin 1 | Proliferation and differentiation |
| | factor receptor 2 (HER3)) | Proliferation associated protein 2G4 (PA2G4) | Oncogenesis (overexpression) |
| | | (EBP1 or ErbB3 binding protein 1) | |
| | | Phosphatidylinositol 3 kinase regulatory | |
| | | subunit alpha (PIK3R1) | |
| | | Regulator of G protein signaling 4 (RGP4) | |
| | ErbB-4 (human epidermal growth | heparin-binding EGF-like growth factor (HB- | Mutations in the RTK have |
| | factor receptor 2 (HER4)) | EGF) | been associated with cancer |
| | | betacellulin | |
| | | epiregulin | |
| | | Neuregulin 1 | |
| | | Neuregulin 2 | |
| | | Neuregulin 3 | |
| | | Neuregulin 4 | |
| RTK class II | Insulin receptor | Insulin | inducing glucose uptake |
| (Insulin receptor family) | | insulin-like growth factor 1 (IGF-1)/ | |
| | | somatomedin C | |
| | | insulin-like growth factor 2 (IGF-2) | |
| RTK class III | PDGFRα | PDGF A/B/C and D | Fibrosis |
| (Platelet derived growth | PDGFRβ | PDGF A/B/C and D | cancer |
| factor (PDGF) receptor | | | |
| family) | | | |
| | Mast/stem cell growth factor | Stem cell factor (SCF)/c-kit ligand/steel | Oncogenesis |
| | receptor (SCFR)/c-Kit/CD117 | factor | Cell survival, proliferation, |
| | | | differentiation |
| | Colony stimulating factor 1 | Colony stimulating factor 1 | Production, differentiation and |
| | receptor/(CD 115)/macrophage | | function of macrophages |
| | colony-stimulating factor receptor | | |
| | (M-CSFR) | | |
| | Cluster of differentiation antigen | Flt3 ligand (FLT3L) | Expressed on surface of many |
| | 135 (CD 135)/ | | hematopoietic progenitor cells |
| | Fms-like tyrosine kinase 3 (FLT-3) | | Mutated in acute myeloid |
| | | | leukemia |
| | | | Cell survival |
| | | | Proliferation |
| | | | differentiation |
| RTK class IV | Fibroblast growth factor receptor-1 | Fibroblast growth factor 1-10 | Wound healing |
| (FGF receptor family) | (CD331) | | Embryonic development |
| | Fibroblast growth factor receptor-2 | | angiogenesis |
| | (CD332) | | |
| | Fibroblast growth factor receptor-3 | | |
| | (CD333) | | |
| | Fibroblast growth factor receptor-4 | | |
| | (CD334) | | |
| | Fibroblast growth factor receptor-6 | | |
| RTK class V | VEGFR1 | VEGF-A | Mitogenesis |
| (VEGF receptor family) | | VEGF-B | |
| (membrane bound or | VEGFR2 | VEGF-A | Cell migration |
| soluble depending on | | VEGF-C | Vasculogenesis |
| alternative splicing) | | VEGF-D | angiogenesis |
| | | VEGF-E | |
| | VEGFR3 | VEGF-C | |
| | | VEGF-D | |
| RTK class VI | Hepatocyte growth factor receptor | Hepatocyte growth factor | Deregulated in certain |
| (Hepatocyte growth factor | (GHFR) (encoded by MET or | | malignancies, leads to |
| receptor family) | MNNG HOS transforming gene). | | angiogenesis |
| | | | Stem cells and progenitor cells |
| | | | express |
| | | | Mitogenesis, morphogenesis |
| RTK class VII | Tropomyosin-receptor kinases | Neurotrophins | Regulate synaptic strength and |
| (Trk receptor family) | (Trk) | Nerve growth factor (TrkA) | plasticity in the mammalian |
| | TrkA | Brain-derived neurotrophic factor (BDNF) | nervous system |
| | TrkB | (TrkB) | |
| | TrkC | Neurotrophin-3 (NT3) (TrkC) | |
| RTK class VIII | EphA | Ephrin-A (Ephrin-A1-5) | Embryonic development |
| (Ephrin (Eph) receptor | (1, 2, 3, 4, 5, 6, 7, 8, 9, 10) | | Axon guidance |
| family) | | | |
| | EphB (1, 2, 3, 4, 5, 6) | Ephrin-B (1-4 and ephrin-B6) | Formation of tissue boundaries |
| | | | Retinopic mapping |
| | | | Cell migration |
| | | | Cell segmentation |
| | | | Angiogenesis |
| | | | Cancer |
| RTK class IX | Tyrosine-protein kinase receptor | Tensin-like C1 domain containing | epithelial-to-mesenchymal |
| (AXL receptor family) | UFO (AXL) | phosphatase (TENC1) | transition-induced regulator of |
| | | | breast cancer metastasis |
| | | | Regulation of cell migration |
| RTK class X | Leukocyte receptor tyrosine kinase | Insulin receptor substrate 1 (IRS- | Apoptosis |
| (Leukocyte receptor | (LTK) | 1) | Cell growth and differentiation |
| tyrosine kinase (LTK) | | Src homology 2 domain containing protein | |
| family) | | (Shc) | |
| | | Phosphatidylinositol 3-kinase regulatory | |
| | | subunit alpha (PIK3R1) | |
| RTK class XI | Tyrosine kinase with | Angiopoietin 1 (Tie2 agonist) | Promotion of angiogenesis |
| (TIE receptor family) | immunoglobulin-like and EGF-like | Angiopoietin 2 (Tie2 antagonist) | TIE1 has a proinflammatory |
| | domains (TIE) 1 | Angiopoietin 3 (Tie2 antagonist) | effect and may play a role in |
| | TIE 2 | Angiopoietin 4 (Tie2 agonist) | atherosclerosis (Chan et al. |
| | | | (2008). Biochem. Biophys. |
| | | | Res. Commun. 371, pp. 475- |
| | | | 479. |
| RTK class XII | ROR-1 (neurotrophic tyrosine | Wnt ligands (ROR-2) | ROR-1 modulates neurite |
| (Receptor typrosine kinase- | kinase, receptor-related 1 | | growth in the central nervous |
| like orphan receptors | (NTRKR1) | | system. |
| (ROR) family) | ROR-2 | | |
| RTK class XIII | DDR-1 (CD167a) | Various types of collagen | DDR-1 is overexpressed in |
| (discoidin domain receptor | DDR-2 | | breast, ovarian, esophageal and |
| (DDR) family) | | | pediatric brain tumors |
| RTK class XIV | Rearranged during transfection | Glial cell line-derived nuerotrophic factor | Loss of function associated |
| (RET receptor family) | (RET) proto-oncogene | (GDNF) family ligands | with Hirschsprung's disease |
| | 3 different isoforms (51, 43, 9) | | Gain of function mutations |
| | | | associated with various types |
| | | | of cancer (e.g., medullary |
| | | | thyroid carcinoma, multiple |
| | | | endocrine neoplasias type 2A |
| | | | and 2D) |
| RTK class XV | Tyrosine-protein kinase-like 7 | No ligand has been identified | Development |
| (KLG receptor family) | (PTK7)/CCK-4 | | Oncogenesis (colon cancer, |
| | | | melanoma, breast cancer, acute |
| | | | myeloid leukemia) |
| | | | Wnt pathway regulation |
| | | | Angiogenesis |
| RTK class XVI | RYK receptor (different isoforms | Wnt ligands | Stimulating Wnt signaling |
| (Related to receptor | due to alternative splicing) | | pathways such as regulation of |
| tyrosine kinase(RYK) | | | axon pathfinding |
| receptor family) | | | |
| RTK class XVII | Muscle-Specific kinase (MuSK) | Agrin (nerve-derived proteoglycan) | Formation of the |
| (Muscle-Specific kinase | receptor | | neuromuscular junction |
| (MuSK) receptor family) | | | |

In one embodiment, the RTK is a platelet derived growth factor receptor (PDGFR), e.g., PDGFRα. PDGFs are a family of soluble growth factors (A, B, C, and D) that combine to form a variety of homo- and hetero-dimers. These dimers are recognized by two closely related receptors, PDGFRα and PDGFRβ, with different specificities. In particular, PDGFα binds selectively to PDGFRα and has been shown to drive pathological mesenchymal responses in fibrotic diseases, including pulmonary fibrosis, liver cirrhosis, scleroderma, glomerulosclerosis, and cardiac fibrosis (see Andrae et al. (2008). Genes Dev. 22, pp. 1276-1312, incorporated by reference herein in its entirety). It has also been shown that constitutive activation of PDGFRα in mice leads to progressive fibrosis in multiple organs (Olson et al. (2009). Dev. Cell 16, pp. 303-313, incorporated by reference herein in its entirety). Thus, therapies that inhibit PDGFRα have high potential for the treatment of fibrosis, a condition that complicates up to 40% of diseases, and represents a huge unmet medical problem in the aging population. Although antibodies (Imatinib and Nilotinib) have been explored as inhibitors of PDGFRα, each has significant off-target effects on other central RTKs, including c-kit and Flt-3, resulting in numerous side effects. Thus, while Imatinib and Nilotinib can effectively inhibit PDGFRα and PDGFRβ, their side effects make them unacceptable in the treatment of fibrotic diseases, highlighting the potential for highly specific antibody inhibitors. The present invention overcomes this problem by providing in one embodiment, antibodies with greater PDGFRα specificity, as compared to Imatinib and Nilotinib.

PDGFRα has been previously established as a target for the treatment of fibrosis. Two anti-human PDGFRα mAb antagonists entering early clinical trials for the treatment of cancer are in development (see, e.g., Shah et al (2010). Cancer 116, pp. 1018-1026, incorporated by reference herein in its entirety). The methods provided herein facilitate the identification of an effector cell secretion product that binds to the PDGFRα. In a further embodiment, the secretion product blocks the activity of both human and murine PDGFRα in both cancer and fibrosis models.

One embodiment of the extracellular effect assay to determine whether an effector cell secretion product binds to PDGFRα is based on the use of suspension cell lines (e.g., 32D and Ba/F3) that are strictly dependent on the cytokine IL-3 for survival and growth, but can be cured of this “IL-3 addiction” through the expression and activation of nearly any tyrosine kinase. This approach was first used by Dailey and Baltimore to evaluate the BCR-ABL fusion oncogene and has been used extensively for high-throughput screening of small molecule tyrosine kinase inhibitors (see, e.g., Warmuth et al. (2007). Curr. Opin. Oncology 19, pp. 55-60; Daley and Baltimore (1988). Proc. Natl. Acad. Sci. U.S.A. 85, pp. 9312-9316, each incorporated by reference in their entireties for all purposes). To monitor signaling, PDGFRα and PDGFRβ (both human and mouse forms) are expressed in 32D cells (readout cells), a murine hematopoietic cell line that does not naturally express either receptor. This allows for separation of each pathway, something that is otherwise difficult since both receptors are often co-expressed. Expression of human PDGFRα/β in 32D cells has been previously confirmed to give a functional PDGF-induced mitogenic response (Matsui et al. (1989). Proc. Natl. Acad. Sci. U.S.A. 86, pp. 8314-8318, incorporated by reference in its entirety). In the absence of IL-3, 32D cells do not divide at all, but PDGF stimulation of the cells expressing the RTK relieves the requirement for IL-3 and gives a rapid mitogenic response that is detectable by microscopy. The detectable response, in one embodiment, is cell proliferation, a morphological change, increased motility/chemotaxis, or cell death/apoptosis in the presence of an antagonist. An optical multiplexing method, in one embodiment, is used to simultaneously measure the inhibition/activation of both PDGFRα and PDGFRβ responses in one of the devices provided herein. In another embodiment, inhibition/activation of both PDGFRα and PDGFRβ responses in one of the devices provided herein is measured by two extracellular assays, carried out serially in the same assay chamber.

Full length cDNA for human/mouse PDGFRα and PDGFRβ (Sino Biological), in one embodiment, is expressed in 32D cells (ATCC; CRL-11346) using modified pCMV expression vectors that also include an IRES sequence with either GFP or RFP, thereby making two types of “readout cells,” each distinguishable by fluorescent imaging. The readout cells are characterized to optimize medium and feeding conditions, determine the dose response to PDGF ligand, and to characterize the morphology and kinetics of response. The use of suspension cells (such as 32D or Ba/F3) provides the advantage that single cells are easily identified by image analysis, and are also physically smaller (in projected area) than adherent cells so that a single chamber can accommodate ≥100 readout cells before reaching confluence. In another embodiment, instead of 32D cells, Ba/F3 cells, another IL-3 dependent mouse cell line with similar properties to 32D are used as readout cells. Both 32D and Ba/F3 cells are derived from bone marrow, grow well in medium optimized for ASCs, and secrete IL-6 which is a critical growth factor for the maintenance of ASCs (see, e.g., Cassese et al. (2003). J. Immunol. 171, pp. 1684-1690, incorporated by reference in its entirety herein).

Preclinical models for evaluating the role of PDGFRα in fibrosis have been developed and can be used in an extracellular effect assay. Specifically, two models of cardiac fibrosis, discussed below, can be employed. The first is based on ischemic damage (isoproterenol-induced cardiac damage; ICD) and the second is based on coronary artery ligation-induced myocardial infarction (MI). Upon damage, the fibrotic response is initiated by the rapid expansion of PDGFRα+/Sca1+ positive progenitors, accounting for over 50% of the cells proliferating in response to damage followed by differentiation of these progeny into matrix producing PDGFRα low/Sca1low myofibroblasts. Gene expression by RT-qPCR demonstrates expression of multiple markers related to fibrotic matrix deposition, including α-smooth muscle actin (αSMA) and collagen type I (Col1), which are detectable in (Sca1+) progenitors, but are substantially up-regulated in the differentiated population. In one embodiment, a cell population is identified that comprises an effector cell that secretes a monoclonal antibody that attenuates progenitor expansion leading to reduced fibrosis. This extracellular effect assay is carried out by monitoring two independent markers: early proliferation of Sca1+/PDGFRα+ progenitors cells and ColI-driven GFP. Specifically, following MI, fibrotic responses are characterized by GFP expression first in PDGFRα+/Sca1+ progenitors, and later, with increased intensity, in the emerging myofibroblast population.

The extracellular effect assay, in one embodiment, is a GPCR extracellular effect assay, for example, GPCR binding, agonism or antagonism. As described herein, the extracellular effect need not be attributable to every cell in the population, or even multiple cells. Rather, the methods provided herein allow for the detection of an extracellular effect of a single effector cell, when the effector cell is present in a heterogeneous population comprising tens to hundreds of cells (e.g., from about 10 to about 250 cells, or from about 10 to about 100 cells), or comprising from about 2 to about 50 cells, e.g., from about 2 to about 10 cells.

GPCRs are a superfamily of seven transmembrane receptors that includes over 800 members in the human genome. Each GPCR has its amino terminus located on the extracellular face of the cell and the C-terminal tail facing the cytosol. On the inside of the cell GPCRs bind to heterotrimeric G-proteins. Upon agonist binding, the GPCR undergoes a conformational change that leads to activation of the associated G-protein. Approximately half of these are olfactory receptors with the rest responding to a gamut of different ligands that range from calcium and metabolites to cytokines and neurotransmitters. The present invention, in one embodiment, provides a method for selecting one or more ASCs that have an extracellular effect on a GPCR. The extracellular effect assays can employ any GPCR, so long as it can be expressed on a readout particle, e.g., readout cell, or accessory particle, e.g., accessory cell.

The type of G-protein that naturally associates with the specific GPCR dictates the cell signaling cascade that is transduced. For Gq coupled receptors the signal that results from receptor activation is an increase in intracellular calcium levels. For Gs coupled receptors, an increase in intracellular cAMP is observed. For Gi coupled receptors, which make up 50% of all GPCRs, activation results in an inhibition of cAMP production. For embodiments where the effector cell property is activation of a Gi coupled GPCR, it is sometimes necessary to stimulate the readout cell(s) with a nonspecific activator of adenylyl cyclase. In one embodiment, the adenyl cyclase activator is forskolin. Thus, activation of the Gi coupled receptor by one or more effector cells will prevent forskolin induced increase in cAMP. Forskolin, accordingly, can be used as an accessory particle in one or more GPCR extracellular effect assays provided herein.

The present invention, in one embodiment, provides means for determining whether an effector cell (e.g., an ASC) within a cell population exhibits an extracellular effect on a GPCR. The GPCR is present on one or more readout particles in an assay chamber and the extracellular effect in one embodiment, is binding to the GPCR, for example, a demonstrated affinity or specificity, inhibition or activation. The GPCR may be a stabilized GPCR, such as one of the GPCRs made by the methods of Heptares Therapeutics (stabilized receptor, StaR® technology). The effector cell (e.g., ASC), in one embodiment, is present as a single cell, or in a homogeneous or heterogeneous cell population within an assay chamber. In one embodiment, the methods and devices provided herein are used to identify one or more cell populations each comprising one or more ASCs that secrete one or more antibodies that demonstrate an extracellular effect on one of the GPCRs set forth in Table 3A and/or Table 3B, or one of the GPCRs disclosed in International PCT Publication WO 2004/040000, incorporated by reference in its entirety. For example, in one embodiment, the GPCR belongs to one of the following classes: class A, class B, class C, adhesion, frizzled.

In another embodiment, the extracellular effect is an effect on an endothelial differentiation, G-protein-coupled (EDG) receptor. The EDG receptor family includes 11 GPCRs (S1P1-5 and LPA1-6) that are responsible for lipid signalling and bind lysophosphatidic acid (LPA) and sphingosine 1-phosphate (S1P). Signalling through LPA and SP regulates numerous functions in health and disease, including cell proliferation, immune cell activation, migration, invasion, inflammation, and angiogenesis. There has been little success in generating potent and specific small molecule inhibitors to this family, making mAbs a very attractive, alternative. In one embodiment, the EDG receptor is S1P3 (EDG3), S1PR1 (EDG1), the latter of which has been shown to activate NF-κB and STAT3 in cancers including breast, lymphoma, ovarian, and melanoma, and plays a key role in immune cell trafficking and cancer metastasis (Milstien and Spiegel (2006). Cancer Cell 9, pp. 148-15, incorporated by reference in its entirety herein). A monoclonal antibody that neutralizes the SP ligand (Sonepcizumab) has recently completed phase II trials for treatment of advanced solid tumours (NCT00661414). In one embodiment, the methods and devices provided herein are used to identify and isolate an ASC that secretes an antibody with greater affinity than Sonepcizumab, or an antibody that inhibits SP to a greater extent than Sonepcizumab. In another embodiment, the extracellular effect is an effect on the LPA2 (EDG4) receptor. LPA2 is overexpressed in thyroid, colon, stomach and breast carcinomas, as well as many ovarian tumors, for which LPA2 is the primary contributor to the sensitivity and deleterious effects of LPA.

In one embodiment, cell populations are assayed for whether one or more exhibits an extracellular effect on a chemokine receptor, present on a readout particle. In a further embodiment, the chemokine receptor is C-X-C chemokine receptor type 4 (CXCR-4), also known as fusin or CD184. CXCR4 binds SDF1α (CXCL12), a strong chemotactic for immune cell recruitment also known as C-X-C motif chemokine 12 (CXCL12). DNA immunizations were used to generate 92 hybridomas against this target, 75 of which exhibited different chain usage and epitope recognition (Genetic Eng and Biotech news, August 2013), indicating that hybridoma selections capture only a small portion of available antibody diversity. Signalling through the CXCR4/CXCL12 axis has been shown to play a central role in tumour cell growth, angiogenesis, cell survival and to be implicated in mediating the growth of secondary metastases in CXCL12-producing organs like liver and bone marrow (Teicher and Fricker (2010). Clin. Cancer Res. 16, pp. 2927-2931, incorporated by reference herein in its entirety).

In another embodiment, cell populations are screened for their ability to exert an effect on the chemokine receptor CXCR7, which was recently found to bind SDF1α. Unlike CXCR4 which signals through canonical G protein coupling, CXCR7 signals uniquely through the β-arrestin pathway.

In another embodiment, the GPCR is a protease activated receptor (PAR1, PAR3, and PAR4), which is a class of GPCRs activated by thrombin-mediated cleavage of the exposed N-terminus and are involved in fibrosis. In yet another embodiment, the GPCR is one of the GPCRs in Table 3A or Table 3B, below.

Cell lines that express GPCRs, engineered to provide a readout of binding, activation or inhibition, are commercially available, e.g., from Life Technologies (GeneBLAzer® and Tango™ cell lines), DiscoveRx, Cisbio, Perkin Elmer, and are amenable for use in the GPCR extracellular effect assays described herein, e.g., as readout cells.

In one embodiment, a GPCR from one of the following receptor families is expressed on one or more readout cells, and an extracellular effect is measured with respect to one or more of the following GPCRs: acetylcholine receptor, adenosine receptor, adreno receptor, angiotensin receptor, bradykinin receptor, calcitonin receptor, calcium sensing receptor, cannabinoid receptor, chemokine receptor, cholecystokinin receptor, complement component (C5AR1), corticotrophin releasing factor receptor, dopamine receptor, endothelial differentiation gene receptor, endothelin receptor, formyl peptide-like receptor, galanin receptor, gastrin releasing peptide receptor, receptor ghrelin receptor, gastric inhibitory polypeptide receptor, glucagon receptor, gonadotropin releasing hormone receptor, histamine receptor, kisspeptin (KiSS1) receptor, leukotriene receptor, melanin-concentrating hormone receptor, melanocortin receptor, melatonin receptor, motilin receptor, neuropeptide receptor, nicotinic acid, opioid receptor, orexin receptor, orphan receptor, platelet activating factor receptor, prokineticin receptor, prolactin releasing peptide, prostanoid receptor, protease activated receptor, P2Y (purinergic) receptor, relaxin receptor, secretin receptor, serotonin receptor, somatostatin receptor, tachykinin receptor, vasopressin receptor, oxytocin receptor, vasoactive intestinal peptide (VIP) receptor or the pituitary adenylate cyclase activating polypeptide (PACAP) receptor.

TABLE 3A
GPCRs amenable for expression in Readout Cells
or as Part of a Stabilized Readout Particle.

| | Human gene | |
|---|---|---|
| Family | symbol | Human Gene Name |
| Calcitonin receptors | CALCR | calcitonin receptor |
| Calcitonin receptors | CALCRL | calcitonin receptor-like |
| Corticotropin-releasing factor receptors | CRHR1 | corticotropin releasing hormone |
| | | receptor 1 |
| Corticotropin-releasing factor receptors | CRHR2 | corticotropin releasing hormone |
| | | receptor 2 |
| Glucagon receptor family | GHRHR | growth hormone releasing hormone |
| | | receptor |
| Glucagon receptor family | GIPR | gastric inhibitory polypeptide |
| | | receptor |
| Glucagon receptor family | GLP1R | glucagon-like peptide 1 receptor |
| Glucagon receptor family | GLP2R | glucagon-like peptide 2 receptor |
| Glucagon receptor family | GCGR | glucagon receptor |
| Glucagon receptor family | SCTR | secretin receptor |
| Parathyroid hormone receptors | PTH1R | parathyroid hormone 1 receptor |
| Parathyroid hormone receptors | PTH2R | parathyroid hormone 2 receptor |
| VIP and PACAP receptors | ADCYAP1R1 | adenylate cyclase activating |
| | | polypeptide 1 (pituitary) receptor |
| | | type I |
| VIP and PACAP receptors | VIPR1 | vasoactive intestinal peptide receptor |
| | | 1 |
| VIP and PACAP receptors | VIPR2 | vasoactive intestinal peptide receptor |
| | | 2 |
| Adenosine | ADORA1 | Adenosine A1 receptor |
| Adenosine | ADORA2A | Adenosine A2 receptor |
| Adenosine | ADRB3 | Adenosine 3 receptor |
| Chemokine | CXCR1 | C-X-C chemokine receptor 1 |
| Chemokine | CXCR2 | C-X-C chemokine receptor 2 |
| Chemokine | CXCR3 | C-X-C chemokine receptor 3 |
| Chemokine | CXCR4 | C-X-C chemokine receptor 4 |
| Chemokine | CXCR5 | C-X-C chemokine receptor 5 |
| Chemokine | CXCR6 | C-X-C chemokine receptor 6 |
| Chemokine | CXCR7 | C-X-C chemokine receptor 7 |
| Chemokine | CCR1 | C-C chemokine receptor type 1 |
| Chemokine | CCR2 | C-C chemokine receptor type 2 |
| Chemokine | CCR3 | C-C chemokine receptor type 3 |
| Chemokine | CCR4 | C-C chemokine receptor type 4 |
| Chemokine | CCR5 | C-C chemokine receptor type 5 |
| Chemokine | CCR6 | C-C chemokine receptor type 6 |
| Chemokine | CCR7 | C-C chemokine receptor type 7 |
| Chemokine | CMKLR1 | Chemokine receptor-like 1 |
| Complement component | C5AR1 | Complement component AR1 |
| | | receptor |
| Lysophospholipid (LPL) receptor | LPAR1 | lysophosphatidic acid receptor 1 |
| Lysophospholipid (LPL) receptor | LPAR2 | lysophosphatidic acid receptor 2 |
| Lysophospholipid (LPL) receptor | LPAR3 | lysophosphatidic acid receptor 3 |
| Lysophospholipid (LPL) receptor | LPAR4 | lysophosphatidic acid receptor 4 |
| Lysophospholipid (LPL) receptor | LPAR5 | lysophosphatidic acid receptor 5 |
| Lysophospholipid (LPL) receptor | LPAR6 | lysophosphatidic acid receptor 6 |
| Lysophospholipid (LPL) receptor | SIPR1 | sphingosine-1-phosphate receptor 1 |
| Lysophospholipid (LPL) receptor | SIPR2 | sphingosine-1-phosphate receptor 2 |
| Lysophospholipid (LPL) receptor | SIPR3 | sphingosine-1-phosphate receptor 3 |
| Lysophospholipid (LPL) receptor | SIPR4 | sphingosine-1-phosphate receptor 4 |
| Lysophospholipid (LPL) receptor | SIPR5 | sphingosine-1-phosphate receptor 5 |

TABLE 3B
GPCRs amenable for expression in Readout Cells or as Part of a Stabilized Readout Particle.

| GPCR | |
|---|---|
| (Gene symbol) | Ligand(s) |
| 5-hydroxytryptamine 1A receptor (HTR1A) | 5-hydroxytryptamine |
| 5-hydroxytryptamine 1B receptor (HTR1B) | 5-hydroxytryptamine |
| 5-hydroxytryptamine 1D receptor (HTR1D) | 5-hydroxytryptamine |
| 5-hydroxytryptamine 1e receptor (HTR1E) | 5-hydroxytryptamine |
| 5-hydroxytryptamine 1F receptor (HTR1F) | 5-hydroxytryptamine |
| 5-hydroxytryptamine 2A receptor (HTR2A) | 5-hydroxytryptamine |
| 5-hydroxytryptamine 2B receptor (HTR2B) | 5-hydroxytryptamine |
| 5-hydroxytryptamine 2C receptor (HTR2C) | 5-hydroxytryptamine |
| 5-hydroxytryptamine 4 receptor (HTR4) | 5-hydroxytryptamine |
| 5-hydroxytryptamine 5a receptor (HTR5A) | 5-hydroxytryptamine |
| 5-hydroxytryptamine 5b receptor (HTR5BP) | 5-hydroxytryptamine |
| 5-hydroxytryptamine 6 receptor (HTR6) | 5-hydroxytryptamine |
| 5-hydroxytryptamine 7 receptor (HTR7) | 5-hydroxytryptamine |
| Acetylcholine M1 receptor (CHRM1) | acetylcholine |
| Acetylcholine M2 receptor (CHRM2) | acetylcholine |
| Acetylcholine M3 receptor (CHRM3) | acetylcholine |
| Acetylcholine M4 receptor (CHRM4) | acetylcholine |
| Acetylcholine M5 receptor (CHRM5) | acetylcholine |
| Adenosine A1 receptor (ADORA1) | adenosine |
| Adenosine A2A receptor (ADORA2A) | adenosine |
| Adenosine A2B receptor (ADORA2B) | adenosine |
| Adenosine A3 receptor (ADORA3) | adenosine |
| α_{1A}-adrenoceptor (ADRA1A) | Adrenaline, noradrenaline |
| | agonists |
| | cirazoline, desvenlafaxine, etilefrine, |
| | metaraminol, methoxamine, midodrine, |
| | naphazoline, oxymetrazoline, phenylephrine, |
| | synephrine, tetrahydrozoline, xylometazoline |
| | antagonists |
| | alfuzosin, arotinolol, carvedilol, doxazosin, |
| | indoramin, labetalol, moxislyte, |
| | phenoxybenzamine, phentolamine, prazosin, |
| | quetiapine, risperidone, silodosin, tamsulosin, |
| | terazosin, tolazoline, trimazosin |
| α_{1B}-adrenoceptor (ADRA1B) | Adrenaline, noradrenaline |
| α_{1D}-adrenoceptor (ADRA1D) | Adrenaline |
| α_{2A}-adrenoceptor (ADRA2A) | Adrenaline |
| α_{2B}-adrenoceptor (ADRA2B) | Adrenaline, noradrenaline |
| | agonists |
| | salbutamol, bitolterol mesylate, isoproteronol, |
| | levosalbutamol, metaproterenol, formoterol, |
| | salmeterol, terbutaline, clenbuterol, ritodrine |
| | antagonists |
| | butoxamine, Beta blockers |
| α_{2C}-adrenoceptor (ADRA2C) | Adrenaline, noradrenaline |
| β₁-adrenoceptor (ADRB1) | Adrenaline, noradrenaline |
| | nitrosamine 4-(methylnitrosamino)-1-(3- |
| | pyridyl)-1-butanone (NNK) |
| | agonists |
| | denopamine, dobutamine, xamoterol |
| | antagonists |
| | acebutol, atenolol, betaxolol, bisoprolol, |
| | esmolol, metoprolol, nebivolol, vortioxetine |
| β₂-adrenoceptor (ADRB2) | Adrenaline |
| β₃-adrenoceptor (ADRB3) | Adrenaline |
| Angiotensin recptor 1 (AT₁) (AGTR1) | Angiotensisn I, angiotensin II, angiotensin III |
| Angiotensin recptor 2 (AT₂) (AGTR2) | Angiotensisn II, angiotensin III |
| Angiotensin recptor 4 | Angiotensisn IV |
| | (angiotensin II metabolite) |
| Apelin receptor (APLNR) | Apelin-13, apelin-17, apelin-36 |
| Bile acid receptor (GPBAR1) | Chenodeoxycholic acid, cholic acid, |
| | deoxycholic acid, lithocholic acid |
| Bombesin receptor BB₁ (NMBR) | Gastrin-releasing peptide, neuromedin B |
| Bombesin receptor BB₂ (GRPR) | Gastrin-releasing peptide, neuromedin B |
| Bombesin receptor BB₃ (BRS3) | |
| Bradykinin recpetor B1 | bradykinin |
| Bradykinin recpetor B2 | bradykinin |
| Calcium sensing receptor (CaSR) | Calcium |
| | Magnesium |
| G-protein coupled receptor family C group 6 member A | |
| (GPRC6A) | |
| Cannabinoid CB₁ receptor | 2-arachidonoylglycerol anandamide |
| (CNR1) | |
| Cannabinoid CB₂ receptor | 2-arachidonoylglycerol anandamide |
| (CNR2) | |
| C-X-C chemokine receptor type 4 (CXCR2) | Interleukin 8 |
| | Growth-related oncogene-alpha (GRO-α) |
| C-X-C chemokine receptor type 4 (CXCR4) | Stromal cell-derived factor-1 (SDF1) |
| Cholecystokinin A receptor (CCKAR or CCK1) | Cholecystokinin peptide hormones (CCK) |
| Cholecystokinin B receptor (CCKAR or CCK2) | Cholecystokinin peptide hormones (CCK) |
| | Gastrin |
| Cholecystokinin recptor CCK1 (CCKAR) | CCK-33, CCK-4, CCK-8, gastrin-17 |
| Cholecystokinin recptor CCK2 (CCKBR) | CCK-33, CCK-4, CCK-8, gastrin-17 |
| endothelin receptor A (ETA) | Endothelin-1 |
| endothelin receptor B1 (ETB1) | Endothelin-1 |
| | Endothelin-3 |
| endothelin receptor B2 (ETB2) | Endothelin-1 |
| | Endothelin-3 |
| endothelin receptor C (ETC) | Endothelin-1 |
| Frizzled 1 (FZD1) | Wnt-1, Wnt-2, Wnt-3a, Wnt-5a, Wnt-7b |
| Frizzled 2 (FZD2) | Wnt-5a |
| Frizzled 3 (FZD3) | Wnt protein ligand |
| Frizzled 4 (FZD4) | Wnt protein ligand |
| Frizzled 5 (FZD5) | Wnt protein ligand |
| Frizzled 6 (FZD6) | Wnt-3a, Wnt-4, Wnt-5a |
| Frizzled 7 (FZD7) | Wnt protein ligand |
| Frizzled 8 (FZD8) | Wnt protein ligands |
| Frizzled 9 (FZD9) | Wnt protein ligands |
| Frizzled 10 (FZD10) | Wnt protein ligands |
| GABA_{B1} Receptor | Agonist |
| GABA_{B2} Receptor | GABA, Baclofen, gamma-hydroxybutyrate, |
| (B1 and B2 assemble as heterodimer) | phenibut, 3-aminopropylphosphinic acid, |
| | lesogaberan, SKF-97541, CGP-44532 |
| | Allosteric modulator |
| | CGP-7930, BHFF, Fendiline, BHF-177, |
| | BSPP, GS-39783 |
| | Antagonists |
| | 2-OH-saclofen, saclofen, phaclofen, SCH- |
| | 50911, CGP-35348, CGP-52432, SGS-742, |
| | CGP-55845 |
| Gastrin-releasing peptide receptor (GRPR), also | Gastrin releasing peptide |
| referred to as BB₂ | |
| G protein-coupled estrogen receptor 30 (GPR30) | Oestrogen |
| Luteinizing hormone/choriogonadotropin receptor | Luteinizing hormone |
| (LHCGR), also referred to as | Chroinic gonadotropins |
| Lutenizing hormone receptor (LHR) and | |
| lutropin/choriogonadotroptin receptor (LCGR) | |
| Lysophosphatidic acid receptor 1 (LPA1) | Lysophosphatidic acid |
| Lysophosphatidic acid receptor 2 (LPA2) | Lysophosphatidic acid |
| Lysophosphatidic acid receptor 3 (LPA3) | Lysophosphatidic acid |
| Melanocortin 1 receptor (MC1R), also referred o as | Melanocortins (pituitary peptide hormones) |
| melanocyte-stimulating hormone receptor (MSHR), | including adrenocorticotropic hormone |
| melanin-activating peptide receptor and melanotropin | (ACTH) and melanocyte-stimulating hormone |
| receptor | (MSH) |
| Neuromedin B receptor | Neuromedin B |
| Prostaglandin E2 receptor EP2 | Prostaglandin E2 |
| Prostaglandin E2 receptor EP4 | Prostaglandin E2 |
| Protease-activated receptor 1 | Thrombin |
| Protease-activated receptor 2 | Trypsin |
| Protease-activated receptor 3 | Thrombin |
| Protease-activated receptor 4 | Thrombin |
| Smoothened | Sonic hedgehog |
| Thyrotropin receptor (TSH receptor) | Thyrotropin |
| Metabotropic glutamate receptor 1(GRM1) | L-glutamic acid |
| Metabotropic glutamate receptor 2 (GRM2) | L-glutamic acid |
| Metabotropic glutamate receptor 3 (GRM3) | L-glutamic acid |
| Metabotropic glutamate receptor 4 (GRM4) | L-glutamic acid |
| Metabotropic glutamate receptor 5 (GRM5) | L-glutamic acid |
| Metabotropic glutamate receptor 6 (GRM6) | L-glutamic acid |
| Metabotropic glutamate receptor 7 (GRM7) | L-glutamic acid |
| Metabotropic glutamate receptor 8 (GRM8) | L-glutamic acid |
| G protein-coupled receptor 56 (GPR56) | |
| G protein-coupled receptor 64 (GPR64) | |
| G protein-coupled receptor 97 (GPR97) | |
| G protein-coupled receptor 98 (GPR98) | |
| G protein-coupled receptor 110 (GPR110) | |
| G protein-coupled receptor 111 (GPR111) | |
| G protein-coupled receptor 112 (GPR112) | |
| G protein-coupled receptor 113 (GPR113) | |
| G protein-coupled receptor 114 (GPR114) | |
| G protein-coupled receptor 115 (GPR115) | |
| G protein-coupled receptor 116 (GPR116) | |
| G protein-coupled receptor 123 (GPR123) | |
| G protein-coupled receptor 124 (GPR124) | |
| G protein-coupled receptor 125 (GPR125) | |
| G protein-coupled receptor 126 (GPR126) | |
| G protein-coupled receptor 128 (GPR128) | |
| G protein-coupled receptor 133 (GPR133) | |
| G protein-coupled receptor 144 (GPR144) | |
| latrophilin 1 (LPHN1) | |
| latrophilin 2 (LPHN2) | |
| latrophilin 3 (LPHN3) | |

In one embodiment, an effector cell is assayed for an extracellular effect on a readout cell expressing a GPCR by one or more of the assays provided in Table 4. In another embodiment, a readout particle population comprises a vesicle or a bead functionalized with a membrane extracts (available from Integral Molecular), or a stabilized solubilized GPCR (e.g., Heptares). Readout particles are added to chambers via microchannels formed in a top component of the device, or directly to open chambers in the bottom component, prior to bringing top and bottom components together.

GPCRs can be phosphorylated and interact with proteins called arrestins. The three major ways to measure arrestin activation are: (i) microscopy—using a fluorescently labeled arrestin (e.g., GFP or YFP); (ii) enzyme complementation; (iii) using the TANGO™ Reporter system (β-lactamase) (Promega). In one embodiment, the TANGO™ Reporter system is employed in a readout cell or plurality of readout cells. This technology uses a GPCR linked to a transcription factor through a cleavable linker. The arrestin is fused to a crippled protease. Once the arrestin binds to the GPCR, the high local concentration of the protease and the linker result in cleavage of the linker, releasing the transcription factor into the nucleus to activate transcription. The β-lactamase assay can be run on live cells, does not require cell lysis, and can be imaged in as little as 6-hours of agonist incubation.

In one embodiment, a β-arrestin GPCR assay that can be universally used for the detection of antagonists and agonists of GPCR signaling is used in the methods and devices provided herein to identify and an effector cell that secretes a biomolecule that binds to a GPCR (Rossi et al. (1997). Proc. Natl. Acad. Sci. U.S.A. 94, pp. 8405-8410, incorporated by reference in its entirety for all purposes). This assay is based on a β-galactosidase (β-Gal) enzyme-complementation technology, now commercialized by DiscoveRx. The GPCR target is fused in frame with a small N-terminal fragment of the β-Gal enzyme. Upon GPCR activation, a second fusion protein, containing β-arrestin linked to the N-terminal sequences of β-Gal, binds to the GPCR, resulting in the formation of a functional β-Gal enzyme. The β-Gal enzyme then rapidly converts non-fluorescent substrate Di-β-D-Galactopyranoside (FDG) to fluorescein, providing large amplification and excellent sensitivity. In this embodiment, readout cells (with GPCRs) are preloaded with cell-permeable pro-substrate (acetylated FDG) prior to introduction into one or more device chambers. The pro-substrate is converted to cell-impermeable FDG by esterase cleavage of acetate groups. Although fluorescein is actively transported out of live cells, by implementing this assay within an assay chamber the fluorescent product is concentrated, providing greatly enhanced sensitivity over plate-based assays. DiscoveRx has validated this assay strategy, used in microwell format, across a large panel of GPCRs.

In one embodiment, activation of a GPCR by an effector cell is determined by detecting the increase in cytosolic calcium in a readout cell(s). In a further embodiment, the increase in cytosolic calcium is detected with one or more calcium sensitive dyes. Calcium sensitive dyes have a low level of fluorescence in the absence of calcium and undergo an increase in fluorescent properties once bound by calcium. The fluorescent signal peaks at about one minute and is detectable over a 5 to 10 minute window. Thus, to detect activity using fluorescent calcium the detection and addition of the agonist are closely coupled. In order to achieve this coupling, the effector cell is exposed simultaneously to the population of readout cells and the one or more calcium sensitive dyes. In one embodiment, the one or more calcium sensitive dyes is a dye provided in a FLIPR™ calcium assay (Molecular Devices).

The recombinant expressed jellyfish photoprotein, aequorin, in one embodiment, is used in a functional GPCR screen, i.e., an extracellular effect assay where the extracellular effect is the modulation of a GPCR. Aequorin is a calcium-sensitive reporter protein that generates a luminescent signal when a coelenterazine derivative is added. Engineered cell lines with GPCRs expressed with a mitochondrially targeted version of apoaequorin are available commercially (Euroscreen). In one embodiment, the one or more of the cell lines available from Euroscreen is used as a population of readout cells in a method of assessing an extracellular effect of an effector cell (e.g., a variation in an extracellular effect as compared to another cell population or a control value).

In one embodiment, an extracellular effect on a GPCR is measured by using one of the ACTOne cell lines (Codex Biosolutions), expressing a GPCR and a cyclic nucleotide-gated (CNG) channel, as a population of readout cells. In this embodiment, the extracellular effect assay works with cell lines that contain an exogenous Cyclic Nucleotide-Gated (CNG) channel. The channel is activated by elevated intracellular levels of cAMP, which results in ion flux (often detectable by calcium-responsive dyes) and cell membrane depolarization which can be detected with a fluorescent membrane potential (MP) dye. The ACTOne cAMP assay allows both end-point and kinetic measurement of intracellular cAMP changes with a fluorescence microplate reader.

A reporter gene assay, in one embodiment, is used to determine whether an effector cell modulates a particular GPCR (e.g., whether a cell population comprising the effector cell modulates the particular GPCR). In this embodiment, the modulation of the GPCR is the extracellular effect being assessed. A reporter gene assay, in one embodiment, is based on a GPCR second messenger such as calcium (AP1 or NFAT response elements) or cAMP (CRE response element) to activate or inhibit a responsive element placed upstream of a minimal promoter, which in turn regulates the expression of the reporter protein chosen by the user. Expression of the reporter, in one embodiment, is coupled to a response element of a transcription factor activated by signaling through a GPCR. For example, reporter gene expression can be coupled to a responsive element for one of the following transcription factors: ATF2/ATF3/AFT4, CREB, ELK1/SRF, FOS/JUN, MEF2, GLI, FOXO, STAT3, NFAT, NFκB. In a further embodiment, the transcription factor is NFAT. Reporter gene assays are available commercially, for example from SA Biosciences.

Reporter proteins are known in the art and include, for example, β-galactosidase, luciferase (see, e.g., Paguio et al. (2006). “Using Luciferase Reporter Assays to Screen for GPCR Modulators,” Cell Notes Issue 16, pp. 22-25; Dual-Glo™ Luciferase Assay System Technical Manual # TM058; pGL4 Luciferase Reporter Vectors Technical Manual # TM259, each incorporated by reference in their entireties for all purposes), green fluorescent protein (GFP), yellow fluorescent protein (YFP), cyan fluorescent protein (CFP), β-lactamase. Reporter gene assays for measuring GPCR signaling are available commercially and can be used in the methods and devices described herein. For example, the GeneBLAzer® assay from Life Technologies is amenable for use with the present invention.

In one embodiment, overexpression of a G protein in a reporter cell is carried out to force a cAMP coupled GPCR to signal through calcium, referred to as force coupling.

In one embodiment, a Gq coupled cell line is used as a readout cell line in the methods described herein. In one embodiment, the Gq coupled cell line reports GPCR signaling through β-lactamase. For example, the cell-based GPCR reporter cell line GeneBLAzer® can be employed (Life Technologies). The reporter cell line can be division arrested or include normal dividing cells.

cAMP responsive element-binding protein (CREB) is a transcription factor as mentioned above, and is used in one embodiment for a Gs and/or Gi coupled GPCR. In a further embodiment, forskolin is utilized as an accessory particle. The CRE reporter is available in plasmid or lentiviral form to drive GFP expression from SA Biosciences, and is amenable for use with the methods and devices described herein. For example, the assay system available from SA Biosciences in one embodiment is employed herein to produce a readout cell (http://www.sabiosciences.com/reporter_assay_product/HTML/CCS-002G.html). Life Technologies also has CRE-responsive cell lines that express specific GPCRs, and these can be used in the methods described herein as well as readout cells.

In one embodiment, one or more effector cells present in a cell population are assayed for the ability to activate or antagonize a GPCR present on one or more readout cells by detecting the increase or decrease in cAMP levels inside the one or more readout cells. ELISA based assays, homogeneous time-resolved fluorescence (HTRF) (see Degorce et al. (2009). Current Chemical Genomics 3, pp. 22-32, the disclosure of which is incorporated by reference in its entirety), and enzyme complementation can all be used with the devices and assays provided herein to determine cAMP levels in readout cells. Each of these cAMP detection methods requires cell lysis to liberate the cAMP for detection, as it is the cyclic AMP that is actually measured.

Assays for measuring cAMP in whole cells and for measuring adenyl cyclase activity in membranes are commercially available (see, e.g., Gabriel et al. (2003). Assay Drug Dev. Technol. 1, pp. 291-303; Williams (2004). Nat. Rev. Drug Discov. 3, pp. 125-135, each incorporated by reference in their entireties), and are amenable for use in the devices and methods provided herein. That is, cell populations in one or more assay chambers can be assayed according to these methods.

Cisbio International (Codolet, France) has developed a sensitive high-throughput homogenous cAMP assay (HTRF, see Degorce et al. (2009). Current Chemical Genomics 3, pp. 22-32, the disclosure of which is incorporated by reference in its entirety) based on time resolved fluorescence resonance energy transfer technology and can be used herein to screen for an effector cell exhibiting an effect on a GPCR. The method is a competitive immunoassay between native cAMP produced by cells and a cAMP-labeled dye (cAMP-d2). The cAMP-d2 binding is visualized by a MaB anti-cAMP labeled with Cryptate. The specific signal (i.e., energy transfer) is inversely proportional to the concentration of cAMP in the sample, in this case, the amount of cAMP activated in a readout cell by an effector cell or an effector cell secretion product. As cAMP is being measured, readout cells are first lysed to free the cAMP for detection. This assay has been validated for both Gₛ- (β₂-adrenergic, histamine H₂, melanocortin MC₄, CGRP and dopamine D₁) and G_{i/o}-coupled (histamine H₃) receptors. As with the other assays described herein, components, can be introduced directly into chambers of a bottom component, e.g., by exposing the bottom component of the device and pipetting a solution containing the components over the chambers in the bottom layer, or through channels in a second component that flow over the chambers of the bottom component. In one embodiment, delivery of reagents/medium to chambers is achieved via hydrostatic pressure created by a liquid column, by creating a flow using a dispensing instrument such as a pipette, or by exchanging the medium overtop of the bottom component and moving the top component or bottom component up and down to evoke a fluid transfer to the microchambers.

cAMP assay kits based on fluorescence polarization are also available commercially, e.g., from Perkin Elmer, Molecular Devices and GE Healthcare, and each is amenable for use as an extracellular effect assay in the methods and devices provided herein. Accordingly, one embodiment of the present invention comprises selecting an effector cell and/or cell population comprising one or more effector cells based on the result of a cAMP fluorescence polarization assay. The method is used in one embodiment to determine whether the effector cell activates (agonism) or inhibits (antagonism) on a particular GPCR.

In one embodiment, the AlphaScreen™ cAMP assay from Perkin Elmer, a sensitive bead-based chemiluminescent assay requiring laser activation, is used in the devices provided herein to screen for an effector cell having an effect on a readout cell, specifically, the activation or inhibition of a GPCR.

DiscoveRx (http://www.discoverx.com) offers a homogenous high-throughput cAMP assay kit called HitHunter™ based on a patented enzyme (β-galactosidase) complementation technology using either fluorescent or luminescent substrates (Eglen and Singh (2003). Comb Chem. High Throughput Screen 6, pp. 381-387; Weber et al. (2004). Assay Drug Dev. Technol. 2, pp. 39-49; Englen (2005). Comb. Chem. High Throughput Screen 8, pp. 311-318, each incorporated by reference in their entireties). This assay can be used to detect an effector cell displaying an extracellular on a readout cell expressing a GPCR.

Cellular events that result from GPCR receptor activation or inhibition can also be detected to determine an effector cell's property(ies) (e.g., an antibody producing cell's ability to activate or antagonize) on a readout cell. For example, in the case of the Gq coupled receptors, when the GPCR is activated, the Gq protein is activated, which results in the phospholipase C cleavage of membrane phospholipids. This cleavage results in the generation of inositol triphosphates 3 (IP3). Free IP3 binds to its target at the surface of the endoplasmic reticulum causing a release of calcium. The calcium activates specific calcium responsive transcription vectors such as nuclear factor of activated T-cells (NFAT). Thus, by monitoring NFAT activity or expression, an indirect readout of the GPCR in a readout cell is established. See. e.g., Crabtree and Olson (2002). Cell 109, pp. S67-S79, incorporated by reference herein in its entirety.

Once activated, more than 60% of all GPCRs are internalized. Utilizing a tagged GPCR (typically done with a C-terminal GFP tag), the distribution of the receptor in one embodiment, is imaged in the presence and absence of ligand. Upon ligand stimulation, a normally evenly distributed receptor will often appear as endocytosed puncta.

TABLE 4
GPCR functional assays for use with the present invention.

| | | Reagents (accessory | | | |
|---|---|---|---|---|---|
| Assay | Biological measurements | particles) | Basis | Endpoint | Notes |
| Europium-GTP ™ | Membrane-based GPCR | Europium-GTP | Binding of europium-labeled GTP to | Time-resolved | Proximal to |
| binding (Perkin | mediated Guanine | | receptor activated G proteins | fluorescence | receptor |
| Elmer) | nucleotide exchange | | | | activation, |
| | | | | | nonradioactive. |
| AlphaScreen ™ | Cell-based cAMP | cAMP MAb conjugated | cAMP competes with biotinyl-cAMP | Luminescence | High sensitivity, |
| (Perkin Elmer) | accumulation | acceptor bead, | binding to high-affinity streptavidin- | | homogeneous, |
| | | streptavidin-coated donor | coated donor beads, loss of signal due | | amenable to |
| | | beads with | to reduced proximity of acceptor- | | automation, broad |
| | | chemoluminescence | donor bead | | linear range of |
| | | compound, biotinyl- | | | detection |
| | | cAMP | | | |
| Fluorescence | Cell- or membrane-based | cAMP MAb, fluorescent | cAMP competes with Fluor-cAMP | Fluorescence | Homogeneous, |
| polarization (Perkin | cAMP accumulation | cAMP | binding to cAMP MAb, loss of signal | polarization | amenable to |
| Elmer, Molecular | | | due to decrease in rotation and | | automation |
| Devices, GE | | | polarization | | |
| Healthcare) | | | | | |
| HTRF cAMP | Cell-based, cAMP | cAMP MAb conjugated | cAMP competes with acceptor- | Time-resolved | Broad linear |
| (Cisbio) | accumulation | with eurocryptate, | labeled cAMP binding to | fluorescence | range, high |
| | | acceptor molecule | europium-conjugated cAMP MAb, | | signal-to- |
| | | labeled cAMP | loss of signal due to reduced | | noise, |
| | | | europium-acceptor molecule | | homogenous, |
| | | | proximity | | amenable to |
| | | | | | automation |
| HitHunter ™ | Cell-based, cAMP | cAMP MAb, ED-cAMP | cAMP competes with ED-cAMP for | Fluorescence or | Low compound |
| (DiscoveRx) | accumulation | (enzyme fragment dono- | complementation of β-Gal activity | luminescence | interference, |
| | | cAMP conjugate) | with binding of acceptor peptide, | | high sensitivity, |
| | | conjugated peptide, | loss of signal as enzyme | | homogeneous, |
| | | acceptor protein, lysis | complementation is reduced | | amenable to |
| | | buffer | | | automation |
| IP₁ ™ (Cisbio) | Cell-based IP₁ | Europium-conjugated IP₁ | Loss of signal as IP₁ competes for | Time-resolved | Homogeneous, |
| | accumulation | MAb, acceptor labeled | binding of acceptor-labeled IP₁ | fluorescence | can be used for |
| | | IP₁ | binding to europium-MAb | | constitutively |
| | | | | | active |
| | | | | | Gq-coupled GPCRs |
| FLIPR ™ | Cell-based, increases in | Caldium sensitive dye; | Increased fluorescence as intracellular | Fluorescence | Sensitive, |
| (Molecular Devices) | intracellular calcium | caldium-3 | dye binds calcium | | homogeneous, |
| | | | | | amenable to |
| | | | | | automation |
| AequoScreen ™ | Cell-based, increases in | Cell lines expressing | Calcium-sensitive aequorin generates | Luminescence | Sensitive, |
| (EuroScreen) | intracellular calcium | select GPCRs along with | a luminescent signal when a | | homogeneous, |
| | | promiscuous or chimeric | coelenterazine derivative is added | | amenable to |
| | | G proteins and a | | | automation |
| | | mitochondrially targeted | | | |
| | | version of apoaequorin | | | |
| Reporter gene | Cell-based, increases in | Several promoter | GPCR changes in secondary | Fluorescence, | Homogeneous, |
| | reporter gene expression | plasmids and reporters | messengers alter expression of a | luminescence, | amplification |
| | due to increases in | | selected reporter gene | absorbance | of signal |
| | second messengers | | | | |
| | activated by | | | | |
| | GPCR binding | | | | |
| Melanophore | Cell-based, changes in | | Melanosomes aggregate with | Absorbance | Sensitive, |
| (Arena | pigment dispersion | | inhibition of PKA disperse with | | homogeneous, |
| Pharmaceuticals) | | | activation of PKA or PKC | | no cell |
| | | | | | lysis, amenable |
| | | | | | to automation |

Adapted from Thomsen et al. (2005). Current Opin. Biotechnol. 16, pp. 655-665, incorporated by reference herein in its entirety for all purposes.

Readout particle responses can be assessed using instrumentation that enables more rapid imaging in multiple fluorescent channels while maintaining conditions required for cell viability followed by recovery of selected single cells automatically, and the depositing of recovered cells into microwell plates suitable for high-throughput genomic analysis (FIG. 31). In one embodiment, the instrumentation includes computerized control and software automation, an inverted fluorescent microscope equipped with automated x-y translation stages having precision encoders, motorized focus, a high-resolution camera, rapid fluorescent illumination systems, an environmental enclosure to maintain temperature, humidity, and carbon dioxide gas levels, and a robotic micromanipulation robot that can direct a micropipette for recovery of cells from any selected chamber, and an adjacent multiwall-plate into which recovered cells can be deposited. In one embodiment, the instrumentation is used in connection with a two-component microfluidic device that allows for the top of the device to be removed prior to recovery of cells. In one embodiment, the removal of the top component of the device is automated using a robotic manipulator of the instrumentation. In another embodiment, the instrumentation is used to analyze cells within a device comprising an open array and a solid piece is positioned for extrusion of medium over top of the array during imaging. In one embodiment, the instrumentation is used to analyze cells within a device featuring an open array of chambers. In one embodiment, the instrumentation is configured with a device featuring an open array of chambers, wherein each chamber has a height/depth greater than its smallest lateral dimension. In one embodiment, the delivery of reagents into the microfluidic device is automated using solenoid valves or a peristaltic pump. In one embodiment, the instrumentation is used with image analysis software to allow for the rapid determination of chambers that contain effector cells of interest. In one embodiment, the microfluidic, two-component microfluidic, or open array of chambers has at least 10,000 chambers, at least 100,000 chambers, or at least 1,000,000 chambers. In one embodiment, the instrumentation is configured for rapid imaging using a wide-field microscope objective and high-resolution camera so that more than 8 chambers can be imaged in a single image. In one embodiment, the instrumentation is configured for rapid imagine using a wide-field microscope objective and high-resolution camera so that greater than about 10, or greater than about 15, or greater than about 16 chambers can be imaged in a single image. It will be appreciated that the number of chambers that may be imaged in one field of view will depend on the total density of chambers and that a higher density of chambers will allow for greater total imaging speed. In the context of the current invention the maximum chamber density is determined by the required area of a chamber that is needed to accommodate at least one cell and at least one readout particle which may be a cell or a bead. While this minimum size is approximately 10 μm×10 μm=100 μm², in practice it is desirable to have larger chamber areas to facilitate stochastic loading strategies that achieve more than one readout particle per chamber, to provide sufficient area for the dispersion of particles or cells in chambers to facilitate imaging, and to enable assays with larger numbers of cells or particles per chamber. Chamber lateral dimensions of about 25 μm×25 μm, or about 50 μm×50 μm or about 75 μm×75 μm or about 100 μm×100 μm or about 150 μm×150 μm or about 200 μm×200 μm, or about 300 μm×300 μm may be used. If about 25 μm spacing is provided between chambers, this corresponds to chamber densities of about 400/mm² or about 178/mm² or about 100/mm² or about 33/mm² or about 20/mm² or about 9/mm².

### EXAMPLES

The present invention is further illustrated by reference to the following Examples. However, it should be noted that these Examples, like the embodiments described above, are illustrative and are not to be construed as restricting the scope of the invention in any way.

### Example 1—Uniform Loading into Two-Component Microfluidic Device

The efficiency of loading a two-component microfluidic device comprising arrays of individual microfluidic chambers was assessed by loading multiple types of microparticles into the open chambers of the bottom component of the device. The dimensions of the microfluidic chambers were 80 μm×120 μm×160 μm (width, length, height).

Five populations of microparticles, each consisting of 5 μm diameter polystyrene beads labeled with a different concentration of fluorophore, were prepared at even concentrations of approximately 1,000,000 beads per mL. Each of the five populations were mixed together in a single tube at equal volumetric ratios. A 100 μL aliquot of the mixture containing approximately 100,000 total microbeads was introduced over a section of the bottom layer of the two-component microfluidic device having approximately 10,000 total chambers. This corresponded to approximately 10 beads per chamber.

The device was then incubated for 20 minutes to allow the beads to settle into the chambers. Medium in the chambers was then removed and chambers were washed by flowing fresh medium over the array. The second layer of the device was placed over the top of the bottom chamber layer. The 10,000 chambers of the microfluidic chamber array were then imaged in fluorescence and automated image analysis was used to determine the distribution of each bead type within the chambers. This analysis showed a generally uniform distribution of beads across the chambers with the following mean bead number and standard deviation per chamber detected for each bead type:

Type 1=0.53/chamber, SD=0.89;

Type 2=1.46/chamber, SD=1.4;

Type 3=2.45/chamber, SD=1.89;

Type 4=3.90/chamber, SD=2.33;

Type 5=2.89/chamber, SD=1.70.

Manual inspection of a subset of images showed that the low measured frequency of Type 1 beads was primarily due to the failure of the image analysis software to detect this bead type; this was attributable to Type 1 beads having the lowest concentration of fluorescent dye. A qualitative analysis of the spatial uniformity of bead distribution, as determined by visual inspection of the bead seeding density across different regions of the device, indicated that these properties were superior as compared to what is achieved when loading beads into microfluidic chambers through input channels.

### Example 2—Cell Loading into Two-Component Microfluidic Devices

The loading of adherent readout cells was assessed in a two-component device of the invention, and compared to the loading of the same cells into a preassembled microfluidic device fabricated via MSL.

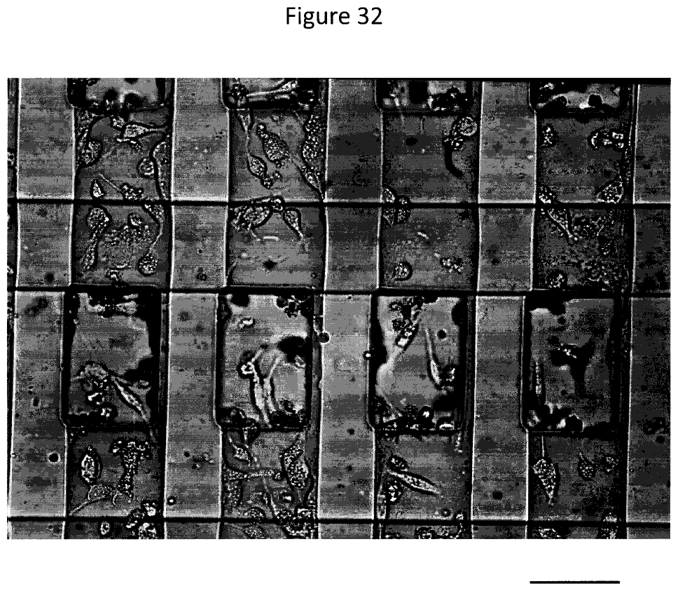

The loading of an adherent cell line (Tango™ CXCR4-bla U2OS) into pre-assembled microfluidic device was assessed by introducing cells through an inlet port and flowing them into the individual chambers through microfluidic channels (channel width 100 μm). This resulted in poor chamber loading uniformity with increased concentrations of cells in the center of the device, low cell loading in the corners of the device, and large variability in the cell loading density between adjacent chambers. Channel clogging due to cell sticking was observed even in medium conditions that included trypsin (FIG. 32). Further, once loaded into chambers and after incubation, cells did not plate down well and showed morphology consistent with stress. Cells had poor viability with most dying over the first 24 hours of culture.

To address the viability issue and improve cell plating in chambers, Tango™ CXCR4-bla U2OS cells were loaded into a second pre-assembled microfluidic device through an inlet port, followed by flowing them into the individual chambers through microfluidic channels. However, in contrast to the first device, the channels and chambers were first exposed to a medium containing poly-lysine. Attempts to load cells through channels pre-coated with poly-lysine resulted in rapid sticking of cells to the channel surfaces, resulting in blocking of the channels and failure of the experiment.

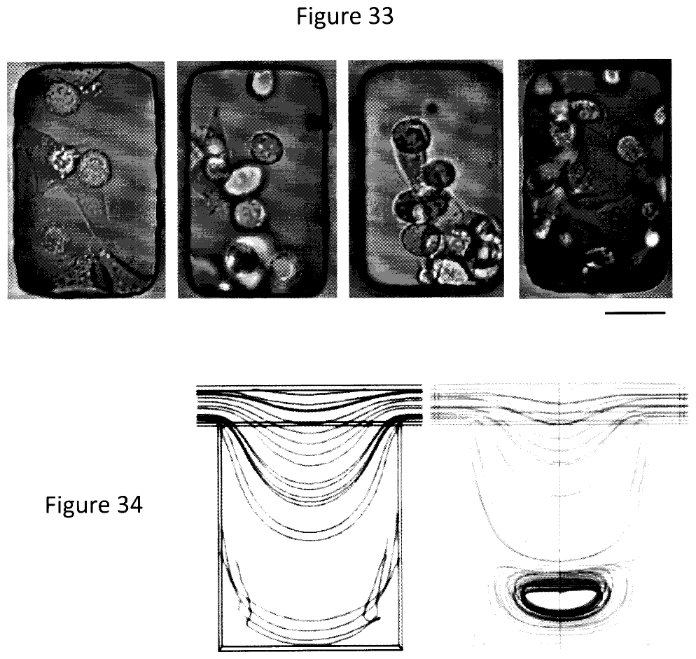

A third experiment was performed that tested the loading of adherent cells in a two-component device. The bottom layer of the device was first coated with medium containing poly-lysine for approximately 10 minutes, followed by a wash with fresh medium that did not contain poly-lysine. Tango™ CXCR4-bla U2OS cells were then loaded into the chambers of the bottom array layer of the device and allowed to plate down over a period of several hours. Chambers (67 μm width×112 μm length) were then washed with fresh medium and the top layer of the device was positioned over the bottom layer to create the two-component device. Imaging of the assembled two-component device showed good uniformity of cell loading with chambers having the number of cells consistent with what would be expected based on seeding density and random positioning across the array, and without any obvious spatial bias in loading density (FIG. 33). Furthermore, the control of cell seeding density, achieved by selection of cell concentration and volume in the loading solution, calculated so that the total number of cells was approximately equal to the product of the desired number of cells per chamber and the total number of chambers, allowed for cell loading control between about 1 cell per chamber to about 20 cells per chamber. For adherent cells, the optimal seeding density was determined by the area of the chamber floor. For example, with the U20S cells and chambers having lateral dimensions of 67 μm×112 μm, an average of approximately 10 to 15 cells per chamber provided good results in terms of uniformity and the ability plate cells. When loaded into two-component devices coated with poly-lysine, cells were found to exhibit normal morphology and were amenable to culture overnight with good viability and without signs of stress.

### Example 3—Impact of Chamber Aspect Ratio During Medium Exchange

A series of experiments were conducted to evaluate the influence of chamber aspect ratio on two-component microfluidic device performance during the exchange of chamber medium. In one experiment, microfluidic devices having chambers with differing aspect ratios (ratio of the depth/height to the minimum lateral dimension), were tested to determine if medium exchange by providing solution over chambers containing cells or beads results in loss of the cells or beads from chambers, or displacement of the cells or beads within chambers.

A device having chambers with dimensions of 100 μm×100 μm×150 μm (depth/height×width×length), corresponding to an aspect ratio of about 1, and channel dimensions of 20 μm×100 μm (height×width) were tested as differing flow rates. Fluid ports at the inlet and the outlet of the device were connected to pressure regulators and the pressure was adjusted to modulate the flow rate through the channels. It was found that medium exchange by flowing through the channels at differential port pressures of 1-3 pounds per square inch (PSI) did not result in a loss of particles from the chambers. The volumetric flow rate per channel at these pressures (channel cross-sections of 15 μm×100 μm) was estimated to be approximately between 0.1 nL/second and 10 nL/second, depending on the section of the device and the channel length across which the pressure was applied. However, at these flow pressures, it was observed that particles (beads or cells) were pushed along the bottom of the chambers in the direction of the flow, causing them to crowd together at the downstream side of the chambers, a result that is acceptable but suboptimal for imaging. At higher pressures of 5 to 9 PSI, the flow caused cells or beads to be lifted out of chambers, a result that negatively impacts the ability to perform multistep assays on cells.

At chamber aspect ratios significantly below 1.0, it was observed that low flow rates (e.g., 1 to 3 PSI) caused a loss of cells or beads from chambers. Based on this result, chambers with higher aspect ratios were tested. It was found that aspect ratios greater than 1.0 allowed for high flow rates to be used without any loss of particles from chambers.

These results may be explained by numerical modeling of flow profiles resulting from different flow rates and chamber aspect ratios. For instance, using chambers with dimensions of 100 μm×100 μm×150 μm (depth×width×length) it was found that the flow is primarily through the top half of the chamber, but that there is non-zero flow down to the bottom of the chamber (FIG. 34, left). Simulations of flow through higher aspect ratio chambers showed improved performance in all cases and in some cases, exhibited qualitatively different flow profiles. For example, simulated flow profiles were calculated through chambers having an aspect ratio of 1.25 and a cylindrical geometry (100 μm diameter and 125 μm depth) (FIG. 34, right). These simulations show that the resulting flow produces a recirculating vortex at the chamber bottom that results in the retention of particles even at low and high flow rates. In accordance with these calculations, it was found that high operating pressures of up to 15 PSI did not result in the loss of particles from chambers, but did result in the motion of particles at the bottom of chambers. Based on these results it was determined that aspect ratios of ≥about 1, should be used in the design of chambers for use in two-component devices.

It is noted that the fluid dynamic behavior in the chamber is determined primarily by the flow velocity at the top of the chamber, and thus the use of such high-aspect ratio chambers is not restricted to two-component devices that include valve structures in the top layer, but can also be used effectively with any two-component device that permits the exchange of medium by flow across the tops of the chambers. These may include two-component devices in which the top component has a single flow channel layer, two-component devices having raised features to create a flow structure when brought together with the bottom layer, or two-component devices that include a slab covering that is positioned in close proximity to, or overtop the bottom layer to allow flow between the two components of the device. Alternatively, bottom components with high-aspect ratio chambers may be used in isolation for certain experiments, i.e., without a top layer. In this case, the high aspect ratio chambers serve to protect particles and cells from transient flow that accompanies liquid exchange over the array, thereby allowing for exchange of medium without displacing particles from chambers. In these embodiments, a fluid reservoir may be placed above the chambers to allow for fluid exchange.

### Example 4—CXCR4 Extracellular Effect Assay

The devices described herein provide a means for performing multistep assays to identify single cells that secrete antibodies that bind specifically to a readout cell type.

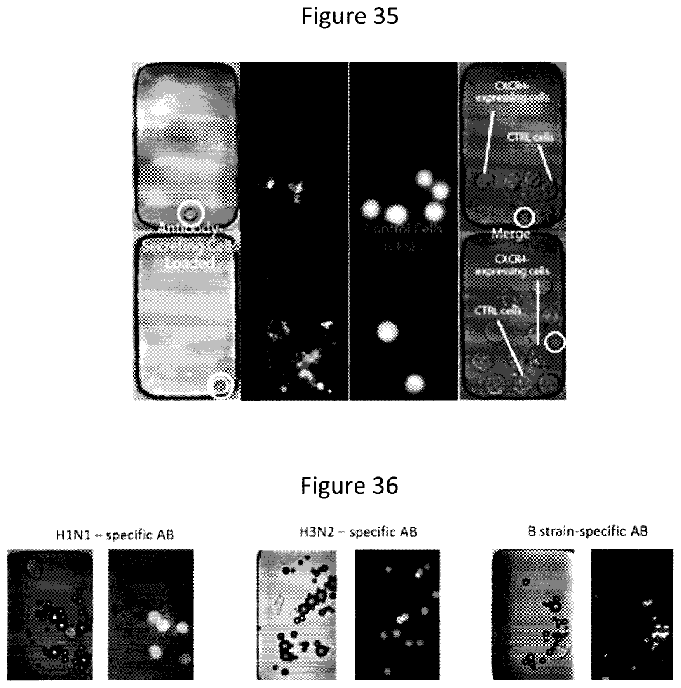

A mouse was immunized with virus-like particles that included the GPCR target CXCR4. ASCs obtained from the spleen of the mouse were loaded into chambers of a two-component microfluidic device, with each chamber having a minimum lateral dimension of approximately 68 μm and a depth of approximately 150 μm, followed by loading of two readout cell populations into the chambers (FIG. 35). One readout cell population included a suspension cell line stably expressing CXCR4. A second readout cell population consisted of the same cell line that does not express CXCR4, and which was fluorescently labeled with the passive dye, carboxyfluorescein succimidyl ester (CFSE). Following loading, the combined cell populations were incubated in the microfluidic device to allow for concentration of secreted antibodies in each chamber and the binding of specific antibodies to the target cells. Following incubation, the chambers were washed by providing medium containing fluorescently labeled secondary antibodies across the device, resulting in the selective staining of cells that express CXCR4, located in chambers with ASCs producing antibodies specific to this target (FIG. 35). The differential staining of cells expressing the target and cells not expressing the target was determined by comparison of the fluorescent signal of the passive dye in a separate channel (FIG. 35, right).

### Example 5—Influenza Antigen Extracellular Effect Assay

The devices described herein provide a means to simultaneously assay antibodies secreted by an individual cell to determine whether they bind to one or more antigens. In the case of influenza, it is useful to identify antibodies that can bind to multiple strains.

Human B-cells were tested for their binding to antigens from three different strains of influenza: H1N1, H3N2, and B strain. Each antigen was coated onto a different bead population having different diameters: H1N1—10 μm, H3N2—5 μm and B strain—3 μm, so that the antigen identity could be determined according to bead size. Both cells and beads were loaded into the bottom layer of a two-component device, with bead densities selected to produce approximately 3 to 20 of each bead type in every chamber, and cell densities selected to have less than a single cell per chamber.

The top component of the device was aligned to the bottom component and the chambers were incubated to concentrate the secreted antibodies and ensure efficient interaction of these antibodies with each of the bead types. Following incubation, the chambers were washed with fresh medium containing a fluorescently labeled secondary antibody by providing the medium across the array of chambers. The fluorescently labelled secondary antibody contained a mixture of anti-human antibodies, with different secondary antibodies labeled in different colors and each being specific to the detection of a different isotype, IgG, IgA and IgM. The chambers were then washed again to remove background fluorescence by providing fresh medium over and across the chambers. The device was imaged to detect chambers having beads that were selectively stained with the secondary antibody. FIG. 36 shows images from three different chambers that were identified to contain single B-cells specific to each of the three different antigens (human IgGs).

### Example 6—Multiplexed Extracellular Effect Assay for h4-1BB Transmembrane Glycoprotein

A bead-based assay was designed to allow for the detection of antibodies that bind to the target human 4-1BB (h4-1BB), a type 2 transmembrane glycoprotein belonging to the tumor necrosis factor (TNF) superfamily. h4-1BB antibodies were also tested for their ability to bind murine 4-1BB. Finally, h4-1BB antibodies were assessed for their ability to block the interaction of 4-1BB with its native ligand h4-1BB ligand.

Mouse antibody secreting cells were obtained from the spleen and bone marrow of mice that had been immunized with soluble h4-1BB. Mice used in this experiment were genetically engineered to produce h4-1BB antibodies with human variable region genes. Beads conjugated to bind the constant region of secreted antibodies were loaded into chambers of the two-component device at an average concentration of about 20 to 30 beads per chamber. ASCs were then loaded into the device and the chambers were incubated for approximately 2 hours to allow for accumulation of secreted antibodies in the chambers and the capture of the secreted antibodies onto the beads.

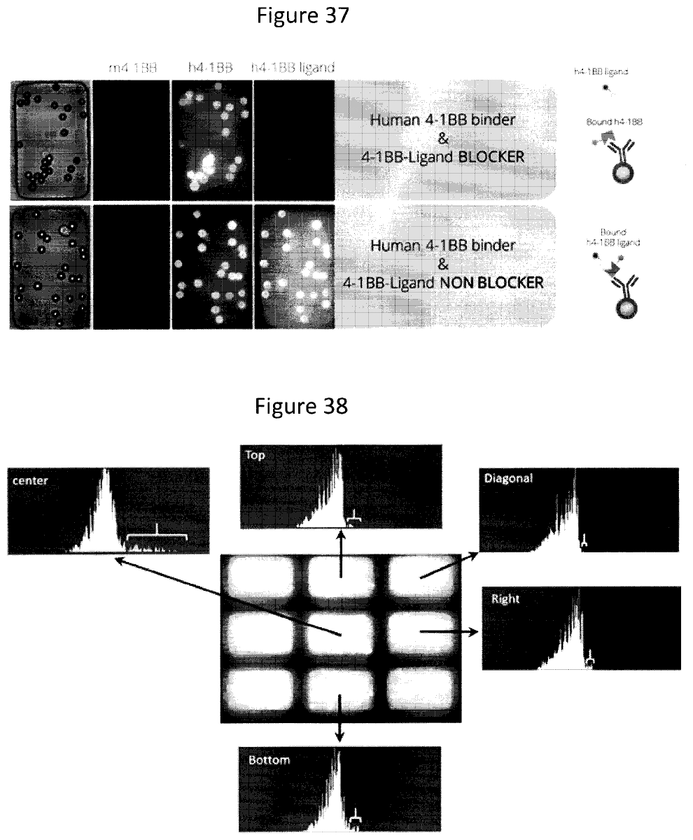

The chambers were then washed by providing a solution containing a mixture of h4-1BB (labeled with a fluorophore) and m4-1BB (labeled with a fluorophore with a second emission profile) over the tops of the chambers. Following incubation, the chambers were imaged in both colors to determine if the secreted antibodies bind to h4-1BB and/or m4-1BB. Results are shown in FIG. 37.

Next, the chambers were again washed by flowing over the tops of the chambers with h4-1BB ligand conjugated with a third fluorophore, which emitted at a third wavelength range. Following incubation, the chambers were imaged in the third fluorescence channel to determine if the bound h4-1BB was still able to bind to the h4-1BB ligand, or if the interaction between h4-1BB and the antibody blocked this interaction (FIG. 37, right). ASCs from chambers that were determined to have desired properties (antibodies that bind to h4-1BB and preserve h4-1BB ligand interaction) were recovered from the chambers by removing the top component of the device and followed by aspiration of the chamber contents with a robotic capillary.

Once aspirated, the chamber contents were deposited into tubes for amplification of the RNA encoding the selected antibodies. The resulting antibody sequences were determined by sequencing. Corresponding DNA inserts were cloned into expression vectors and used for recombinant expression of the antibodies. A subset of the resulting antibodies were then tested to confirm that they exhibit the properties that were determined from the microfluidic screen.

### Example 7—Detection of Antibodies in a One-Component Open Microfluidic Device

The incorporation of a top component in the two-component devices presented here provides several advantages in the analysis of secreted antibodies from single cells. For example, the two-component device offers increased sensitivity by confining secreted antibodies to the volume of a single chamber; facilitates fluid handling steps needed to implement multi-step assays; reduces background fluorescence to increase signal to noise; and prevents of cross-contamination between chambers by diffusion of antibodies.

Nevertheless, the devices presented here may also be used in a format that does not incorporate a top component. In this example, a bottom component (single component) of a device was loaded with microbeads conjugated with an antigen. Next, a population of antibody secreting cells was loaded into the device chambers at a concentration of approximately 1 cell per chamber and was incubated to allow for capture of antigen-specific antibodies on the beads in the chambers containing a cell, and to a lesser extent in the chambers adjacent to the chambers containing a cell. A low concentration (˜10 nM) of fluorescently labeled secondary antibody was then added to the solution overlaying the chambers and incubated for approximately 1 hour, followed by imaging of the chambers. Results are provided in FIG. 38.

Analysis of images showed the presence of groups of chambers in which the pixel intensity of beads was significantly above the background fluorescence produced by the soluble secondary antibody. Analysis of the histograms of pixel intensities in these groups of chambers showed that a central chamber exhibited pixel intensities with the highest values as compared to background, that chambers directly to the bottom and the top of this chamber had the next highest, followed by chambers directly to the right and to the left, and finally by chambers on the diagonal from the central chamber (FIG. 38). These differences in intensity are explained by differences in proximity of the beads in each chamber to the cell producing the antibody (in the central chamber)—it is noted that the array spacing in this experiment was shorter in the y-coordinate as compared to the x-coordinate, which contributed to the higher intensities in the top and bottom chambers (y-coordinate) as compared to the right and left chambers (x-coordinate). From this analysis, detection and recovery of single antibody secreting cells of desired specificity from the central chamber was carried out. Antibody identity was confirmed by sequencing, cloning, and expression or the corresponding antibody sequences.

The detection of antibodies in the single layer format requires that the antibody secreting cells produce sufficient antibody to be detected in the presence of higher background (as compared to when a top component is used in a two-component device) due to the presence of a large volume above the chamber that contains fluorescent molecules (e.g., via the use of a fluid reservoir), and in the presence of antibody escaping by diffusion into adjacent chambers. This was confirmed by the observation that analysis of the same sample using a device format that included a top-component resulted in an increase in the number of specific antibodies that was detected, as well as elimination of adjacent fluorescence due to restriction of diffusion between chambers and an increased signal to noise.

### Example 8—Multiplexed Detection of 5 Antigens

Multiplexed immunizations and screening were performed to isolate rabbit monoclonal antibodies against 5 different antigens. Rabbits were immunized with a mixture of 5 different antigens over a period of approximately 6 weeks. Following immunizations, a sample of blood was obtained from a rabbit that exhibited titers for all 5 antigens and peripheral blood mononuclear cells (PBMCs) were isolated from this sample. The isolated PBMCs containing plasma cells were then loaded into the bottom layer of a microfluidic device that was pre-loaded with a mixture of 5 different bead families (Starfire™ beads, Bangs Laboratories). Each bead family was conjugated with one of the five antigens used to immunize the rabbits, and each family was optically distinguishable by the level of a fluorescent dye included in the bead matrix. Following loading of cells and beads, the two-component device was assembled and the chambers were incubated for approximately 2 hours to allow for concentration of secreted antibody and the efficient capture of specific antibodies on beads having the corresponding antigen. The chambers were then washed with fresh medium containing secondary antibodies labeled with a fluorophore optically different from the fluorophore used in the bead matrix. The microfluidic device was then imaged in the two fluorescent channels and automated real-time image analysis of the first channel was used to automatically segment and identify the beads in each chamber. Image analysis of the images taken in the second fluorescent channel was used to determine if antibodies had bound to each of the different bead types. Results showed that all five antigens were detectable.

### Example 9—Klebsiella pneumonia Binding Extracellular Effect Assay

The devices described here were used for the discovery of fully human antibodies against the bacterial pathogen Klebsiella pneumonia. Antibody secreting cells were obtained from human bone marrow, tonsils, and human blood. For screening of these cells, whole Klebsiella pneumonia was loaded into a microfluidic device at a concentration of approximately 100 bacteria per chamber. Antibody secreting cells were then loaded into the chambers of the device and a top component was aligned to the bottom layer, followed by incubation to allow for accumulation of secreted antibody. Confinement in nanoliter volume chambers allowed for antibodies that recognize an antigen presented on the surface of a bacterium to subsequently bind to the bacterium.

The chambers were then washed by flowing fresh medium containing a mixture of two differentially labeled secondary antibodies, one specific to human IgG and one specific to human IgA, over the tops of the chambers. Imaging confirmed that bacteria were not lost during the flowing of fresh medium. Without wishing to be bound by theory, it is thought that bacteria retention was due to the low flow rate and/or the aspect ratio of approximately 1 of the chambers.

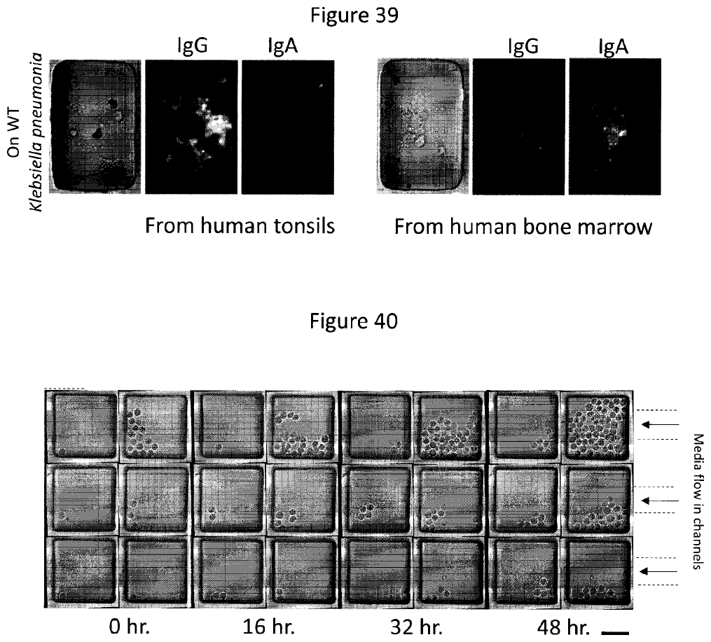

Following a second incubation with the medium containing a mixture of the two differentially labeled secondary antibodies, the microfluidic device was imaged in two colors to identify chambers that contained single antibody secreting cells producing antibodies specific to an antigen on the bacteria, and to determine the isotype of these antibodies (IgG or IgA). Results are provided in FIG. 39.

Following detection, the top component of the microfluidic device was removed and the contents of selected chambers were recovered. These contents were then used as template to amplify the corresponding antibody sequences. These sequences were then synthesized, cloned, and expressed to produce recombinant antibody. A set of the recombinant antibodies were then tested and confirmed to bind to the Klebsiella pneumonia bacterium.

### Example 10—Detection of Monoclonal Antibody in the Presence of Multiple Different ASCs in a Single Chamber

The following experiment was carried out to demonstrate that the devices and methods described herein are amenable for use in assays in which a monoclonal antibody produced by a single cell can be detected and analyzed in a single chamber, when there are multiple other cells, e.g., other antibody secreting cells, in the same chamber.

A human sample was screened for rare antibodies that bind to a bacterial pathogen, Klebsiella pneumonia. A sample of human PBMCs was obtained and cultured under activating conditions that promote the expansion of memory B-cells and differentiation into ASCs. Following activation, the bottom component of a two-component microfluidic device was preloaded with live bacteria and then loaded with cells from the PBMC culture at a concentration of approximately 50 cells per chamber. The device was then assembled with its second component, and incubated to allow for accumulation of secreted antibodies in the chambers, and the interaction of these antibodies with the bacteria. After incubation, the chambers were washed by flowing fresh medium containing a mixture of two differentially labeled secondary antibodies, one specific to human IgG and one specific to human IgA, over the tops of the chambers. Imaging confirmed that bacteria were not lost during the flowing of fresh medium. Without wishing to be bound by theory, it is thought that bacteria retention was due to the low flow rate and/or the aspect ratio of approximately 1 of the chambers.

Following a second incubation with the medium containing the mixture of the two differentially labeled secondary antibodies, the microfluidic device was imaged in two colors to identify chambers that contained single antibody secreting cells producing antibodies specific to an antigen on the bacteria, and to determine the isotype of these antibodies (IgG or IgA). Less than 0.1% of all 90,000 chambers were found to contain an antibody specific for the bacteria, thereby showing that a single antigen-specific antibody secreting cell, present within a population of approximately 50 other cells, was responsible for the positive signal observed in some chambers. Following detection of positive chambers, the top component of the microfluidic device was removed and the contents of the selected chambers were recovered and pooled together to create and enriched population of antibody secreting cells in which the approximate frequency of cells specific to the bacterial was 2% (1 in every 50 cells). This enriched population of cells was then rescreened on a second device at limiting dilution (less than one cell per chamber) and individual antibody secreting cells that secreted antibodies specific to the bacteria were successfully detected and recovered.

### Example 11—Prolonged Microfluidic Culture of Mammalian Cells

The following example was carried out to demonstrate that the two-component devices and methods presented herein allow for the performance of experiments that require the prolonged culture of mammalian cells with high viability. A microfluidic device was loaded with a population of K562 cells. Different sections of the device were loaded at varying numbers of cells per chamber ranging from 1 cell to approximately 20 cells per chamber (chamber dimensions: 200 μm width×200 μm length×140 μm depth). Following loading, the device was imaged to determine the number of cells in each chamber. A subset of device chambers was monitored by bright-field microscopy for a period of 48 hr. to evaluate the expansion and viability of cells in each chamber. Throughout the experiment, the chambers were flushed with fresh medium every 6 hours to ensure the availability of sufficient nutrients in the medium and to remove metabolic products that may inhibit cell growth. It was observed that chambers exhibited robust growth and excellent viability, and that robust cell growth was sustained even in chambers where cells grew to confluence (FIG. 40).

### Example 12—Recovery of Cells from Individual Microfluidic Chambers

The following example was performed to demonstrate that recovery from individual device chambers can be accomplished without cross-contamination and damaging the integrity of the recovered cells.

In this example, a population of human antibody secreting cells (ASCs) was loaded into chambers present on the bottom component of a two-component microfluidic device along with microbeads designed to capture secreted antibodies, followed by alignment of the top component to the bottom component. Cells were loaded at a concentration of less than one cell per chamber. Following incubation, the chambers were washed with fresh medium containing a fluorescently labeled secondary antibody to human IgG. The device was incubated and imaged to detect chambers having either single cells that secreted IgG or control chambers having no cell. The top component of the device was then removed and a robotically controlled micro-capillary was used to recover the contents of 10 chambers, alternating between chambers that contained a single IgG secreting cell and control chambers that contained no cell. The contents of each chamber were deposited in separate microfuge tubes and a RT-PCR reaction was performed to amplify the heavy and light chain variable regions of human antibodies.

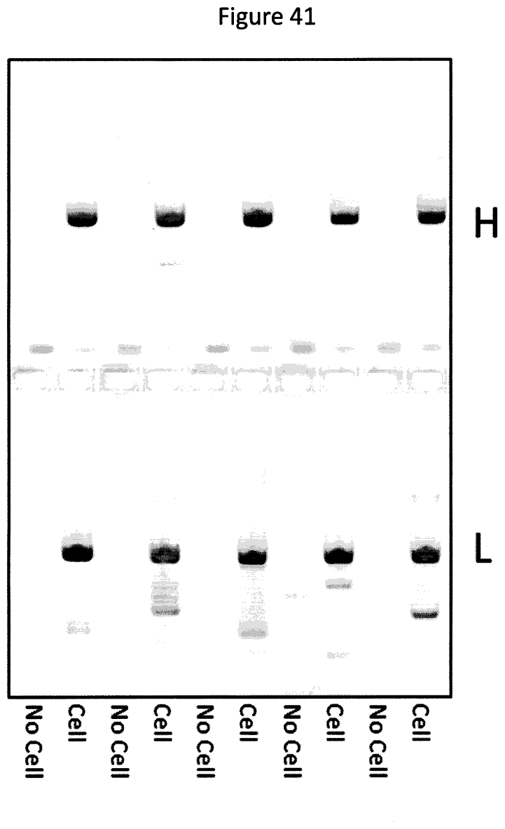

Following amplification, an agarose gel was run to analyze the resulting amplification products. Results showed that the reactions containing template derived from chambers having a single cell, i.e., a single IgG antibody secreting cell, produced clear heavy and light chain bands, and that all reactions that contained no template (control chambers) did not produce a product (FIG. 41). This confirmed that (i) cells were efficiently recovered from the chambers, (ii) there was no significant cross-contamination between chambers, and iii) the recovered cells were intact and contained sufficient RNA to allow for recovery of antibody genes by RT-PCR.

### Example 13—Temporal and Spatial Control of Medium Exchange in Chambers

The following example was performed to demonstrate that the devices and methods presented herein allow for the exercise of spatial and temporal control over the exchange of medium within one or more chambers of the microfluidic device. This control may be particularly useful when conducting experiments in which a functional extracellular effect assay requires high temporal resolution in imaging. For instance, such assays may include monitoring the lysis of a cell exposed to a factor, e.g., an antibody, monitoring translocation of a fluorescent protein within readout cells, monitoring ion channel flux in readout cells in response to a stimulus, monitoring second message calcium flux in response to a stimulation. For example, a microfluidic single cell assay be implemented to identify antibodies that inhibit rapid calcium flux in readout cells in response to addition of an agonist, as measured by a calcium-sensitive fluorescent dye preloaded into the readout cells. In this case, the signal is transient so that imaging of an entire device of more than 10,000 chambers may not provide sufficient temporal resolution. This problem may be overcome by using a two-component microfluidic device in which the top component is designed to enable the selective addition of agonist to subsections of the device under controlled timing so that the chambers can be imaged at a known and suitable time following agonist addition. This may be achieved using a two-component microfluidic device in which the top layer contains valve and channel structures that permit for the flowing of a solution over only a selected subset of chambers. The number of subsections and the number of chambers per subsection will depend on the requirements of the assay and may be designed into the fluidic network. Alternatively, a top component with channels but without valves may be used to achieve the same result by having separate inlets for different sections of the fluidic network that interface with a defined number of chambers. Alternatively, this may be achieved by using a top layer that includes an orifice through which the agonist can be flowed over the chambers and which is not fixed in location relative to the bottom layer, but rather can be translated across the chamber array to exposed different areas. Alternatively, the device may be used without a top layer and a robotically controlled capillary may be used to flow an agonist over different regions of the array. Alternatively, the device may be used without a top layer but may be designed to include partitions that isolate different sub-regions of the device, each having a suitable number of chambers for the assay, and addition of the agonist to each of the different subarrays may be achieved by pipetting onto the subarrays.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A method of identifying a cell population comprising an antibody secreting cell (ASC) displaying an extracellular effect attributable to a secreted antibody of the ASC, comprising:
retaining a plurality of cell populations each comprising from about 10 to about 100 cells in a plurality of different open chambers each having an average aspect ratio (chamber height:minimum lateral dimension) of ≥0.6, wherein the plurality of open chambers is present in a first component of a microfluidic device comprising a first component and optionally a second component, wherein the first component forms a reversible seal with the optional second component,
wherein at least one individual cell population of the plurality optionally comprises one or more ASCs,
wherein the individual open chambers of the plurality, or subset thereof, further comprise a readout particle population comprising one or more readout particles,
incubating the contents of the open chambers,
assaying the plurality of open chambers, or subset thereof, for the presence of the extracellular effect, wherein the readout particle population or a subpopulation thereof provides a direct or indirect readout of the extracellular effect, and
determining, based on the results of the assaying step, whether the one or more ASCs within the at least one individual cell population exhibits the extracellular effect.

2. The method of claim 1, wherein the second component comprises multiple layers fabricated via multilayer soft lithography (MSL).

3.-5. (canceled)

6. The method of claim 1, wherein the one or more ASCs comprise a plasma cell, B-cell, plasmablast, a cell generated through the expansion of memory B-cell, a hybridoma cell, a recombinant cell engineered to produce antibodies, or a combination thereof.

7. (canceled)

8. The method of claim 1, wherein one or more of the readout particle populations comprises one or more readout beads, one or more readout cells, or a combination thereof.

9.-13. (canceled)

14. The method of claim 1, wherein the plurality of readout particle populations, or a subset thereof, comprises one or more readout cells that express a G protein-coupled receptor (GPCR) on its surface or a solubilized GPCR, and
the extracellular effect is antagonism of the GPCR, agonism of the GPCR or binding to the GPCR by an antibody secreted by the one or more ASCs.

15. (canceled)

16. The method of claim 1, wherein the plurality of readout particle populations, or a subset thereof, comprises one or more readout cells that express an ion-channel on its surface, and
the extracellular effect is antagonism of the ion channel, agonism of the ion channel or binding to the ion channel by an antibody secreted by the one or more ASCs.

17.-30. (canceled)

31. The method of claim 1, wherein one or more of the readout particle populations is functionalized with an antigen or epitope.

32. The method of claim 1, wherein one or more of the readout particle populations is functionalized with an antibody binding moiety.

33. The method of claim 32, wherein the antibody binding moiety is Protein A, Protein A/G, Protein G, a monoclonal antibody that binds an immunoglobin, a monoclonal antibody fragment that binds an immunoglobin, a polyclonal antibody that binds an immunoglobin, a polyclonal antibody fragment that binds an immunoglobin, or a combination thereof.

34.-35. (canceled)

36. The method of claim 1, wherein the plurality of cell populations is loaded into the open chambers via hydrostatic pressure created by a liquid column, by a dispensing instrument, or by moving the top component or bottom component up and down to evoke a fluid transfer to the microchambers.

37.-38. (canceled)

39. The method of claim 1, wherein the plurality of readout particle populations, or a subset thereof, is loaded into the open chambers via hydrostatic pressure created by a liquid column, by a dispensing instrument, or by moving the top component or bottom component up and down to evoke a fluid transfer to the microchambers.

40.-43. (canceled)

44. The method of claim 1, wherein the extracellular effect is a binding interaction between an antibody secreted by the one or more ASCs, or subset thereof, to the readout particle population, or subpopulation thereof.

45.-46. (canceled)

47. The method of claim 44, wherein the binding interaction is an antigen-antibody binding specificity interaction, antigen-antibody binding affinity interaction or antigen-antibody binding kinetic interaction.

48. The method of claim 1, wherein the extracellular effect is modulation of apoptosis, modulation of cell proliferation, a change in a morphological appearance of a readout particle, a change in localization of a protein within a readout particle, expression of a protein by a readout particle, neutralization of the biological activity of an accessory particle, cell lysis of a readout cell induced by the ASC, cell apoptosis of the readout cell induced by the ASC, readout cell necrosis, internalization of an antibody, internalization of an accessory particle, enzyme neutralization by the ASC, neutralization of a soluble signaling molecule or a combination thereof.

49. The method of claim 1, further comprising maintaining one or more of the cell populations in one or more of the plurality of chambers in substantially a single plane.

50. The method of claim 49, further comprising maintaining one or more of the readout particle populations in substantially the same plane as the one or more cell populations.

51.-78. (canceled)

79. The method of claim 1, further comprising recovering a cell population from a chamber where the extracellular effect is detected.

80. The method of claim 79, further comprising,
retaining a plurality of cell subpopulations originating from the recovered cell population in a plurality of different open chambers having an average aspect ratio (chamber height:minimum lateral dimension) of ≥0.6, of a microfluidic device comprising a first component and optionally a second component, wherein the first component forms a reversible seal with the optional second component and the plurality of open chambers is present in the first component,
wherein an individual cell subpopulation of the plurality comprises one or more antibody secreting cells (ASCs),
wherein the individual open chambers of the plurality, or subset thereof, further comprise a readout particle population comprising one or more readout particles,
incubating the contents of the chambers,
assaying the plurality of chambers, or subset thereof, for the presence of the extracellular effect, wherein the readout particle population or a subpopulation thereof provides a direct or indirect readout of the extracellular effect, and
identifying, based on the results of the assaying step, a cell subpopulation from amongst the plurality that comprises one or more ASCs that exhibit the extracellular effect.

81. The method of claim 80, wherein the cell subpopulations of the plurality comprise an average of from about 1 cell to about 25 cells.

82.-101. (canceled)

102. The method of claim 1, wherein the second component is present.

103. (canceled)

104. The method of claim 102, wherein the second component comprises multiple layers.

105. (canceled)

106. The method of claim 102, wherein the second component is unpatterned.

107. The method of claim 102, wherein the second component comprises one or more flow channels and one or more push down valve structures.

108.-125. (canceled)
